(19) United States
(12) Patent Application Publication
(10) Pub. No.: US 2011/0172165 A1
(43) Pub. Date: Jul. 14, 2011

(54) MODIFIED LINKERS

(76) Inventors: Peter Artymiuk, Sheffield (GB); Richard A. Ross, Sheffield (GB); Jon Sayers, Chesterfield (GB)

(21) Appl. No.: 12/867,747

(22) PCT Filed: Feb. 18, 2009
(86) PCT No.: PCT/GB2009/000437

§ 371 Date: Dec. 7, 2010

(30) Foreign Application Priority Data
Feb. 19, 2008 (GB) 0802978.7
Nov. 19, 2008 (GB) 0821076.7
Jan. 14, 2009 (GB) 0900539.8

(51) Int. Cl. A61K 38/14; C07K 14/52; C07H 21/04; C12N 15/63; C12N 5/10; C07K 14/535; C12N 1/00; C07K 14/55; C07K 14/555

(52) U.S. Cl. 514/20.9; 530/395; 536/23.2; 435/320.1; 435/325; 530/351; 435/419; 435/243

(57) ABSTRACT

We describe modified peptide linkers that function to link at least first and second polypeptides wherein said modified peptide linker comprises a motif for the addition of a sugar moiety.

The invention relates to modified peptide linkers that function to link at least first and second polypeptides wherein said modified peptide linker comprises a motif for the addition of a sugar moiety.

Glycosylation is the addition of a sugar pendent group to a protein, polypeptide or peptide which alters the activity and/or bioavailability of the protein, polypeptide or peptide. The process is either co-translational or post-translational and is enzyme mediated. Two types of glycosylation exist; N-linked glycosylation to an asparagine side chain and O-linked glycosylation to a serine or threonine amino acid side chain. N-linked glycosylation is the most common post-translational modification and is carried out in the endoplasmic reticulum of eukaryotic cells. N-linked glycosylation can be of two main types; high mannose oligosaccharides which are two N-acetylglucosamines and complex oligosaccharides which include other types of sugar groups. A peptide motif contained in glycosylated polypeptides is Asn-X-Ser or Asn-X-Thr where X is any amino acid except proline. This is catalyzed by the enzyme oligosaccharyl transferase (OT); see Yan & Lennarz J. Biol. Chem., Vol. 280 (5), 3121-3124 (2005) OT catalyzes the transfer of an oligosaccharyl moiety (Glc3Man9GlcNAc2) from the dolichol-linked pyrophosphate donor to the side chain of an Asn. A pentasaccharide core is common to all N-linked oligosaccharides and serves as the foundation for a wide variety of N-linked oligosaccharides.

O-linked glycosylation is less common. Serine or threonine residues are linked via their side chain oxygen to sugars by a glycosidic bond. Usually N-acetyl glucosamine is attached in this way to intracelluar proteins.

It is known that glycosylation alters the function of biologically active proteins. For example, WO01/36001 describes a modified interferon y conjugate comprising an N-linked glycosylation which is purported to have improved pharmacokinetics (PK) and reduced immunogenicity. WO2004/020578 discloses N-linked glycosylation of IL-20 genetically engineered variants that preferentially signal through IL20 receptor. WO2005/070138 describes O-linked glycosylation of therapeutic peptides, for example G-CSF that are not typically glycosylated to reduce immunogenicity and improve bioavailability. Further examples of glycosylated G-CSF are disclosed in

WO2007/108882 which is expressed in transgenic cells and which alters the G-CSF glycosylation pattern. WO2007/022799 describes a process for manufacturing recombinant glycosylated interferon in serum free conditions to provide a differentially glycosylated protein when compared to native glycosylated interferon β and which is manufactured at elevated amounts. WO2007/084441 discloses Follicle Stimulating Hormone mutants with increased glycosylation which have improved PK and its use in the treatment of fertility. WO2007/136752 discloses processes for the homogeneous glycosylation of erythropoietin, a protein that contains four carbohydrate chains; three N-linked glycosylated sites and one O-linked. A problem associated with the production of glycosylated recombinant protein is the heterogeneity in glycosylation. Further examples of attempts to improve the PK of erythropoietin are described in WO00/32772, WO01/02017 and WO03/029291.

In our co-pending application WO01/96565 we described fusion proteins with improved PK and agonist activity which are fusions of cytokines to the extracellular domains of their cognate receptors. The cytokine and extracellular domains are linked to one another via a flexible peptide linker that allows the respective domains to move relative to each other. An example is growth hormone (GH).

GH binds sequentially with two membrane bound growth hormone receptors (GHR) via two separate sites on GH referred as site 1 and site 2. Site 1 is a high affinity binding site and site 2 a low affinity site. A single GH molecule binds 1 GHR via site 1. A second GHR is then recruited via site 2 to form a GHR:GH:GHR complex. The complex is then internalised and activates a signal transduction cascade leading to changes in gene expression. The extracellular domain of GHR exists as two linked domains each of approximately 100 amino acids (SD-100), the C-terminal SD-100 domain (b) being closest to the cell surface and the N-terminal SD-100 domain (a) being furthest away. It is a conformational change in these two domains that occurs on hormone binding with the formation of the trimeric complex GHR-GH-GHR. GH chimeric fusion proteins administered to rats have a 300-times reduced clearance compared to native GH and single administration promoted growth for 10 days far superior to that seen with native GH. The growth hormone fusion forms a reciprocal, head-to-tail dimer that provides a reservoir of inactive hormone as occurs naturally with GH and its binding protein. A further example is disclosed in our co-pending application WO03/070765 which describes modified GH fusion proteins that include modifications to site 1 and site 2 in GH and which act as antagonists of GH receptor activation.

Further examples of novel chimeric fusion proteins are described in unpublished patent applications directed to: growth hormone (US60/951,122; 0717985.6); modified growth hormone (UK0719818.7; US60/979,010) leptin (UK0715216.8; US60/956,360); erythropoietin (UK0715126.9; US60/956,319); granulocyte colony stimulating hormone (UK0715133.5; US60/956,303); interferon (UK0715383.6; US60/956,343); interleukin UK0715557.5; US60/956,372; IGF-1 (UK0715213.5; US60/956,333) and prolactin (UK0724654.9; UK 0724656.4); and peptide hormone chimeras (UK0725201.8); the contents of which is incorporated by reference in their entirety.

In WO03/034275 we described fusion proteins comprising oligomers (e.g. dimers, trimers) of cytokines, for example growth hormone and leptin, which act as agonists of receptor mediated signal transduction. The cytokine moieties are also linked to one another via flexible peptide linkers. The oligomers although active do not have the improved PK when compared to chimeric fusion proteins.

Peptide linker molecules that link polypeptides are known in the art. For example, WO2006/010891 describes rigid and semi-rigid peptide linker molecules that link amongst other proteins growth hormone in tandem which comprise peptides with helical regions to restrict flexibility. In EPO 573 551 is described a serine rich peptide linking molecule (Ser Ser Ser Ser Gly)ₓ (wherein is x can be between 1-10 copies) useful in linking domains in fusion proteins and single chain antibody fragments to improve solubility and resist protease digestion. In WO88/09344 a glycine rich linking molecule is described (Gly Gly Gly Gly Ser) that links heavy and light chain antibody fragments. Peptide linking molecules are therefore established as a means by which polypeptide domains can be linked to one another to form functional complexes that have improved activity and/or PK.

We disclose a modified peptide linking molecule that is modified to include a motif for the attachment of sugar moieties thereby forming a glycosylated peptide linker between at least two active polypeptide binding moieties.

According to an aspect of the invention there provided a fusion polypeptide comprising first and second polypeptides linked indirectly by a peptide linker wherein said peptide linker is modified to include at least one motif for the addition of at least one sugar moiety.

In a preferred embodiment of the invention said first polypeptide is a ligand and said second polypeptide is a cognate receptor to which said ligand can bind; preferably the extracellular domain comprising or consisiting a domain that binds said ligand.

In an alternative preferred embodiment of the invention said ligand is a chemokine.

The term “chemokine” refers to a group of structurally related low-molecular weight factors secreted by cells having mitogenic, chemotactic or inflammatory activities. They are primarily cationic proteins of 70 to 100 amino acid residues that share four conserved cysteine residues. These proteins can be sorted into two groups based on the spacing of the two amino-terminal cysteines. In the first group, the two cysteines are separated by a single residue (C-x-C), while in the second group they are adjacent (C-C). Examples of member of the ‘C-x-C’ chemokines include but are not limited to platelet factor 4 (PF4), platelet basic protein (PBP), interleukin-8 (IL-8), melanoma growth stimulatory activity protein (MGSA), macrophage inflammatory protein 2 (MIP-2), mouse Mig (m119), chicken 9E3 (or pCEF-4), pig alveolar macrophage chemotactic factors I and II (AMCF-I and -II), pre-B cell growth stimulating factor (PBSF),and IP10. Examples of members of the ‘C-C’ group include but are not limited to monocyte chemotactic protein 1 (MCP-1), monocyte chemotactic protein 2 (MCP-2), monocyte chemotactic protein 3 (MCP-3), monocyte chemotactic protein 4 (MCP-4), macrophage inflammatory protein 1 α (MIP-1-α), macrophage inflammatory protein 1β (M1-1-β), macrophage inflammatory protein 1-γ (MIP-1-γ), macrophage inflammatory protein 3 α (MIP-3-α, macrophage inflammatory protein 3 β (MIP-3-β), chemokine (ELC), macrophage inflammatory protein-4 (MIP-4), macrophage inflammatory protein 5 (MIP-5), LD78 β, RANTES, SIS-epsilon (p500), thymus and activation-regulated chemokine (TARC), eotaxin, I-309, human protein HCC-1/NCC-2, human protein HCC-3.

In a further preferred embodiment of the invention said ligand is a pro-angiogenic polypeptide.

A number of growth factors have been identified which promote/activate endothelial cells to undergo angiogenesis. These include vascular endothelial growth factor (VEGF A); VEGF B, VEGF C, and VEGF D; transforming growth factor (TGFb); acidic and basic fibroblast growth factor (aFGF and bFGF); and platelet derived growth factor (PDGF). VEGF is an endothelial cell-specific growth factor which has a very specific site of action, namely the promotion of endothelial cell proliferation, migration and differentiation. VEGF is a complex comprising two identical 23 kD polypeptides. VEGF can exist as four distinct polypeptides of different molecular weight, each being derived from an alternatively spliced mRNA. bFGF is a growth factor that functions to stimulate the proliferation of fibroblasts and endothelial cells. bFGF is a single polypeptide chain with a molecular weight of 16.5 Kd. Several molecular forms of bFGF have been discovered which differ in the length at their amino terminal region. However the biological function of the various molecular forms appears to be the same. bFGF is produced by the pituitary gland.

In a preferred embodiment of the invention said pro-angiogenic polypeptide is selected from the group consisting of: VEGF A, VEGF B, VEGF C, VEGF D, TGFb, aFGF and bFGF; and PDGF.

In a further preferred embodiment of the invention said ligand is a growth factor.

Insulin-like growth factor 1 (IGF1) and its cognate receptor IGF1R are, in combination with human GH, essential for normal growth and development. Additionally IGF1R has also been implicated in malignant transformation (Baserga et al 1997). The IGF1, IGF2 and insulin receptors are closely related and IGF1R can also be activated by IGF2. IGF1 R consists of an alpha chain of approximately 740 residues disulphide linked to a transmembrane beta chain (90kDa) which includes the cytoplasmic tyrosine kinase domain. Two alpha chains are disulphide linked so that the receptor forms an alpha2:beta2 tetramer on the membrane (Hubbard and Till, 2000). The alpha chain consists of several domains: two L domains, L1 (residues 1-150) and L2 (residues 300-460) are largely responsible for binding the hormone; the L domains are separated by a Cys-rich domain (151-299), and followed by fibronectin Type III domains (460-700) (Baserga R, Hongo A, Rubini M, Prisco M & Valentis B (1997) “The IGF-1 receptor in in cell growth, transformation and apoptosis” Biochim Biophys Acta 1332: F105-F126); Hubbard S B & Till, J H (2000) “Protein tyrosine kinase structure and function.” Annu. Rev. Biochem. 59:373-398).

In a preferred embodiment of the invention said ligand is insulin-like growth factor 1.

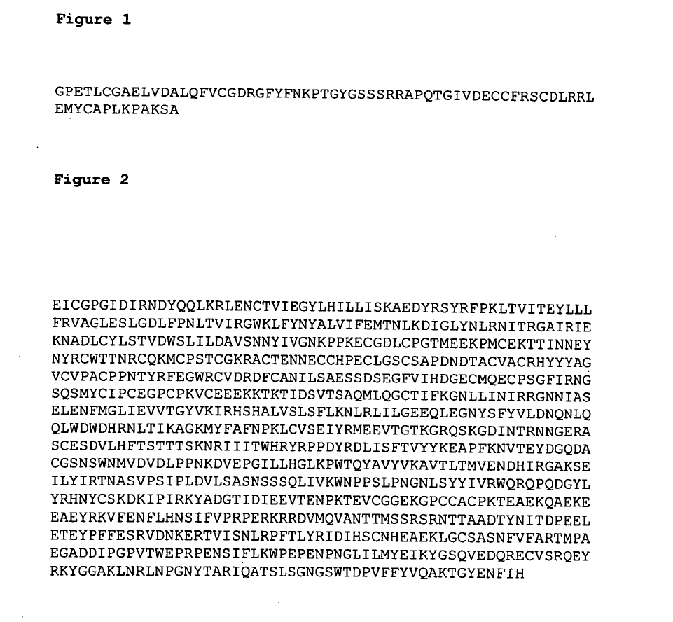

In a preferred embodiment of the invention said ligand is human insulin-like growth factor 1 and is represented by the amino acid sequence in FIG. 1.

In a preferred embodiment of the invention said receptor domain comprises or consists of an IGF-1 receptor polypeptide as represented by the amino acid sequence in FIG. 2.

In a preferred embodiment of the invention said ligand is insulin-like growth factor 2.

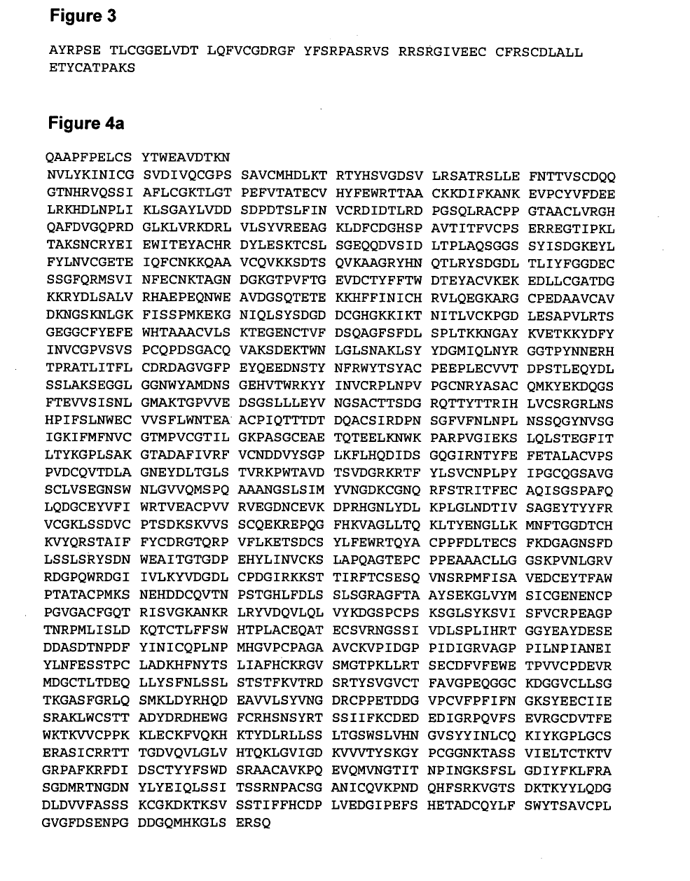

In a preferred embodiment of the invention said ligand is human insulin-like growth factor 2 and is represented by the amino acid sequence in FIG. 3.

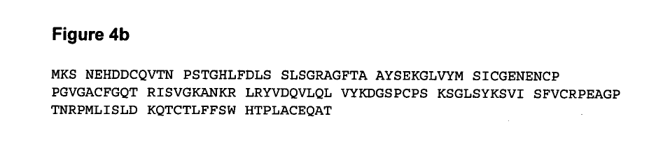

In a preferred embodiment of the invention said receptor domain comprises or consists of an IGF-2 receptor polypeptide as represented by the amino acid sequence in FIG. 4a or 4b.

In a preferred embodiment of the invention said ligand is a cytokine.

Cytokines, are involved in a number of diverse cellular functions. These include modulation of the immune system, regulation of energy metabolism and control of growth and development. Cytokines mediate their effects via receptors expressed at the cell surface on target cells. Cytokine receptors can be divided into three separate sub groups. Type 1 (growth hormone (GH) family) receptors are characterised by four conserved cysteine residues in the amino terminal part of their extracellular domain and the presence of a conserved Trp-Ser-Xaa-Trp-Ser motif in the C-terminal part. The repeated Cys motif is also present in Type 2 (interferon family) and Type III (tumour necrosis factor family).

In a preferred embodiment of the invention said cytokine is selected from the group consisting of: growth hormone; leptin; erythropoietin; prolactin; interleukins (IL) IL-2, IL-3, IL-4, IL-5, IL-6, IL-7, IL-9, IL-10, IL-11, the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin (CT-1); leukocyte inhibitory factor (LIF); interferon type I, II or III.

In a preferred embodiment of the invention said interferon is a type I interferon.

Preferably said type I interferon is selected from the group consisting of: interferon α, interferon β, interferon ε, interferon κ and ω interferon.

In a preferred embodiment of the invention said interferon a is selected from the group consisting of: IFNA 1, IFNA 2, IFNA 4, IFNA 5, IFNA 6, IFNA 7, IFNA 8, IFNA 10, IFNA 13, IFNA 14, IFNA 16, IFNA 17 and IFNA 21.

In an alternative preferred embodiment of the invention said first polypeptide is interferon β and said second polypeptide comprises an interferon β binding domain of an interferon receptor.

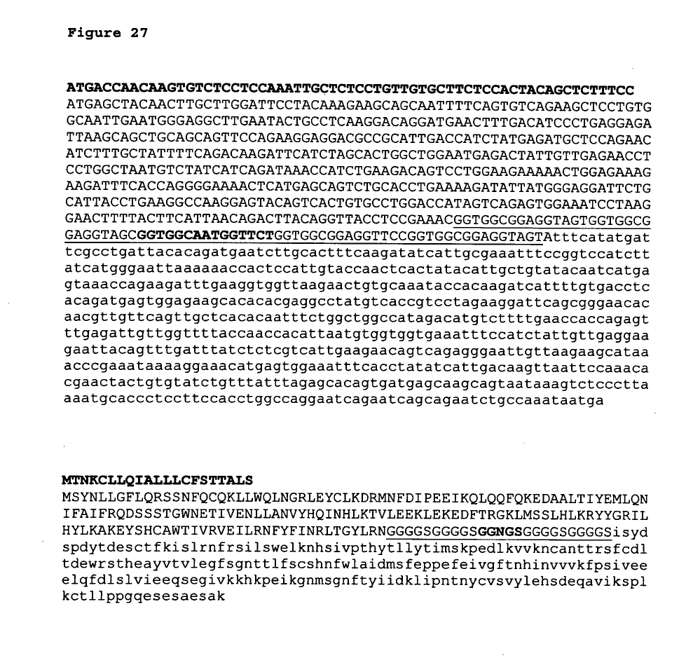

In a preferred embodiment of the invention said fusion polypeptide comprises an amino acid sequence as represented in FIG. 27, 28, 29, 30 or 31.

In an alternative preferred embodiment of the invention said cytokine is selected from the group consisting of: growth hormone; leptin; erythropoietin; prolactin; and G-CSF.

In a preferred embodiment of the invention growth hormone is a human growth hormone polypeptide.

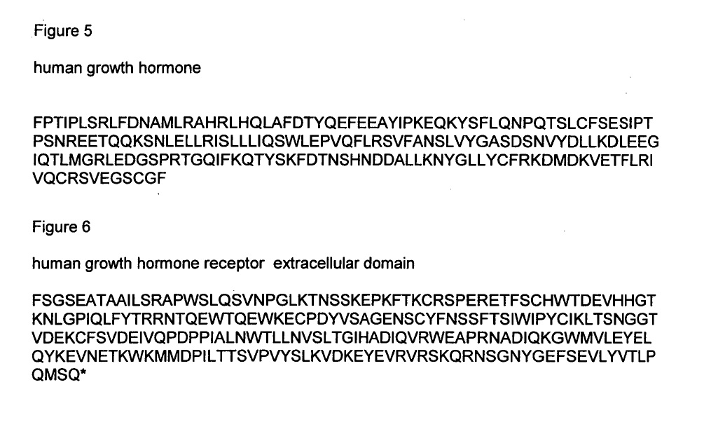

Preferably said human growth hormone polypeptide is represented by the amino acid sequence presented in FIG. 5.

In a preferred embodiment of the invention said growth hormone polypeptide is a modified human growth hormone polypeptide which is modified in at least one receptor binding domain.

In a preferred embodiment of the invention said receptor binding domain of growth hormone is a site one binding domain.

In an alternative preferred embodiment of the invention said receptor binding domain of growth hormone is a site two binding domain.

In a preferred embodiment of the invention said modified growth hormone is modified in both site one and site two.

In a preferred embodiment of the invention said site two modification is to glycine 120 of the amino acid sequence as represented in FIG. 5.

In a preferred embodiment of the invention said site two modification is a substitution of glycine for an amino acid selected from the group consisting of: arginine; alanine; lysine; tryptophan; tyrosine; phenylalanine; and glutamic acid.

In a preferred embodiment of the invention said substitution is glycine 120 for arginine or lysine or alanine. Preferably said substitution is glycine 120 for arginine.

In a preferred embodiment of the invention said receptor domain comprises or consists of a growth hormone receptor polypeptide as represented by the amino acid sequence in FIG. 6.

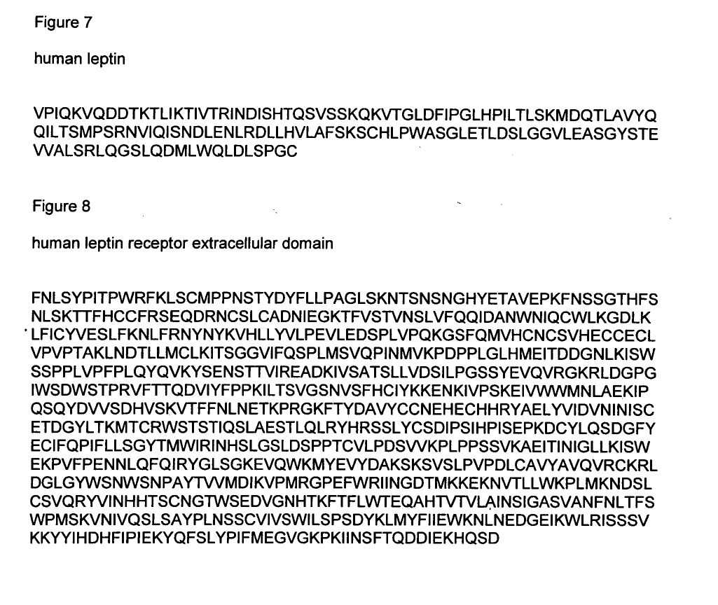

In a preferred embodiment of the invention leptin is human leptin and represented by the amino acid sequence presented in FIG. 7.

In a preferred embodiment of the invention said receptor domain comprises or consists of a leptin receptor polypeptide as represented by the amino acid sequence in FIG. 8.

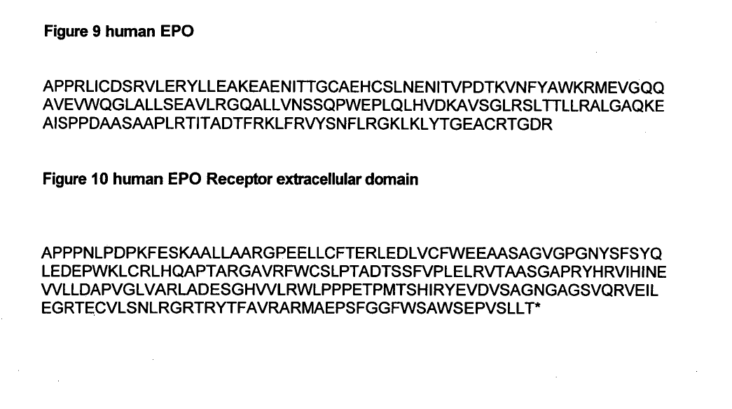

In a preferred embodiment of the invention erythropoietin is human erythropoietin and is represented by the amino acid sequence presented in FIG. 9.

In a preferred embodiment of the invention said receptor domain comprises or consists of an erythropoietin receptor polypeptide as represented by the amino acid sequence in FIG. 10.

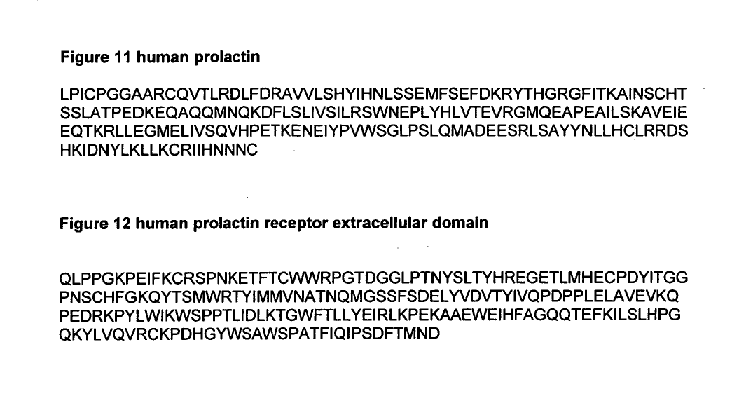

In a preferred embodiment of the invention prolactin is human prolactin and is represented by the amino acid sequence presented in FIG. 11.

In a preferred embodiment of the invention prolactin is a modified human prolactin wherein prolactin is modified in at least one receptor binding domain.

In a preferred embodiment of the invention said modified prolactin polypeptide comprises an amino acid sequence wherein said amino acid sequence is modified at position 129 of human prolactin as represented in FIG. 11.

In a preferred embodiment of the invention said modification is an amino acid substitution. Preferably said substitution replaces a glycine amino acid residue with an arginine amino acid residue.

Preferably said modification further comprises the deletion of at least 9, 10, 11, 12, 13 or 14 amino terminal amino acid residues of the amino acid sequence as represented in FIG. 11.

In a preferred embodiment of the invention said receptor domain comprises or consists of a prolactin receptor polypeptide as represented by the amino acid sequence in FIG. 12.

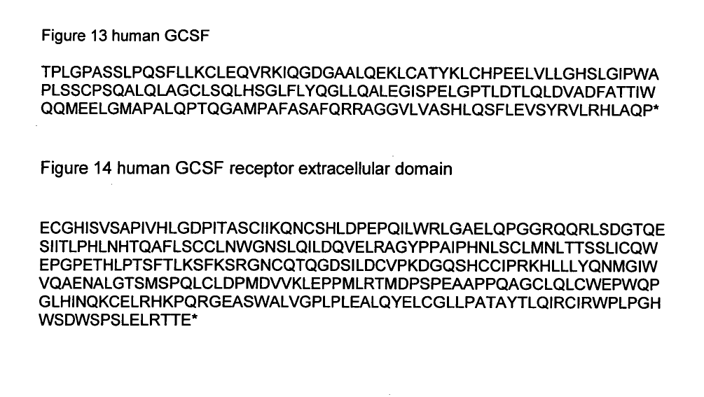

In a further preferred embodiment of the invention G-CSF is human G-CSF and is represented by the amino acid sequence presented in FIG. 13.

In a preferred embodiment of the invention said receptor domain comprises or consists of a G-CSF receptor polypeptide as represented by the amino acid sequence in FIG. 14.

In a preferred embodiment of the invention said ligand is a peptide hormone.

In a preferred embodiment of the invention said peptide hormone is selected from the group consisting of: anti-diuretic hormone; oxytocin; gonadotropin releasing hormone, corticotrophin releasing hormone; calcitonin, glucagon, amylin, A-type natriuretic hormone, B-type natriuretic hormone, ghrelin, neuropeptide Y, neuropeptide YY₃-₃₆, growth hormone releasing hormone, somatostatin; or homologues or analogues thereof.

In a preferred embodiment of the invention said fusion protein comprises growth hormone releasing hormone.

In an alternative preferred embodiment of the invention said fusion protein comprises somatostatin or homologue or analogue thereof; preferably somatostatin is somatostatin 14. Alternatively somatostatin is somatostatin 28.

In a preferred embodiment of the invention said fusion protein comprises the amino acid sequence: AGCKNFFW KTFTSC.

In an alternative preferred embodiment of the invention said fusion protein comprises the amino acid sequence: SANSNPAMAPRERKAGCKNFFW KTFTSC.

In a preferred embodiment of the invention said receptor binding domain is an extracellular receptor binding domain.

In a preferred embodiment of the invention said receptor binding domain comprises a somatostatin binding domain of a somatostatin 1 receptor.

In a preferred embodiment of the invention said receptor binding domain comprises a somatostatin binding domain of a somatostatin 2 receptor. somatostatin binding domain of a somatostatin 3 receptor.

In a preferred embodiment of the invention said receptor binding domain comprises a somatostatin binding domain of a somatostatin 4 receptor.

In a preferred embodiment of the invention said receptor binding domain comprises a somatostatin binding domain of a somatostatin 5 receptor.

In a preferred embodiment of the invention said extracellular receptor binding domain comprises or consists of a somatostatin binding domain.

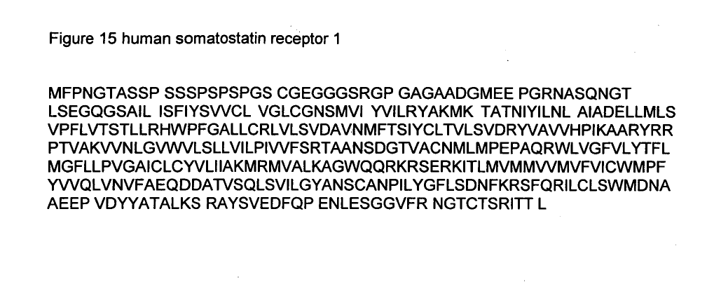

In a preferred embodiment of the invention said extracellular receptor binding domain comprises or consists of a somatostatin binding domain as illustrated by the amino acid sequence in FIG. 15 with reference to table 1.

In an alternative preferred embodiment of the invention said first polypeptide and said second polypeptide is somatostatin 14.

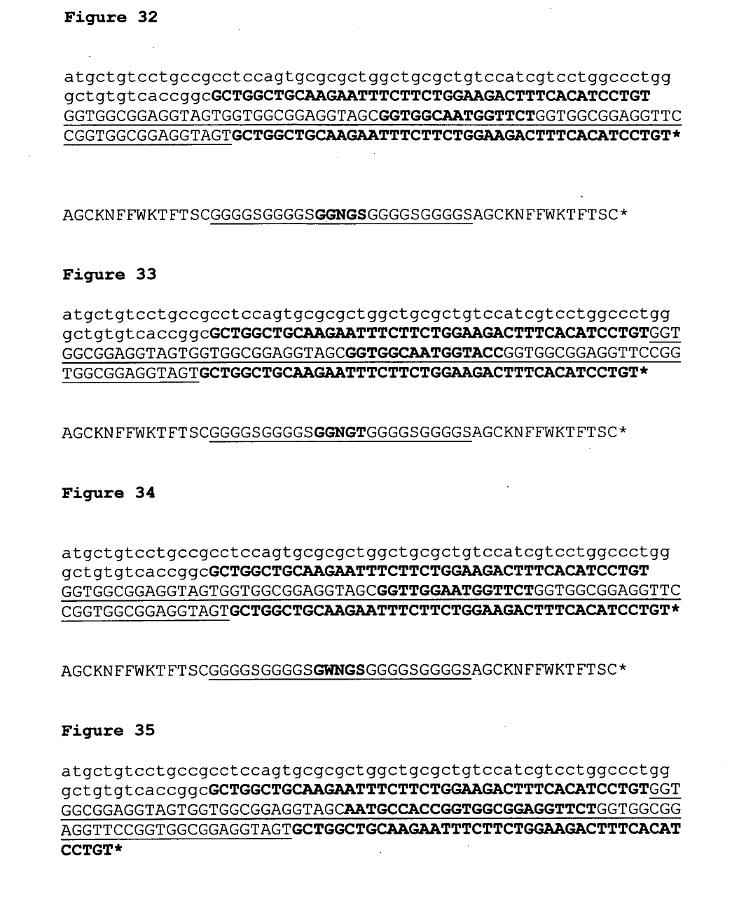

In a preferred embodiment of the invention said fusion polypeptide comprises an amino acid sequence as represented in FIG. 32, 33, 34, 35 or 36.

In a further alternative preferred embodiment of the invention said first and said second polypeptide is somatostatin 28.

In a preferred embodiment of the invention said fusion polypeptide comprises an amino acid sequence as represented in FIG. 38, 39, 40 or 41.

In an alternative preferred embodiment of the invention said first polypeptide is somatostatin 14 and said second polypeptide is somatostatin 28.

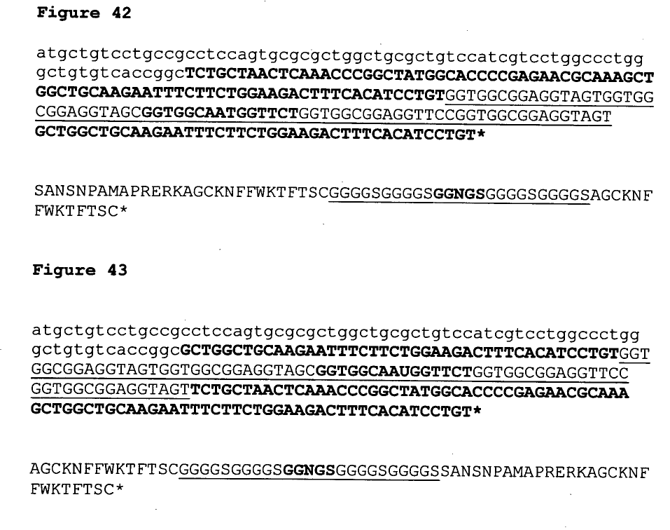

In a preferred embodiment of the invention said fusion polypeptide comprises an amino acid sequence as represented in FIG. 42 or 43.

In an alternative preferred embodiment of the invention said fusion polypeptide comprises of the follicle stimulating hormone (FSH) a subunit.

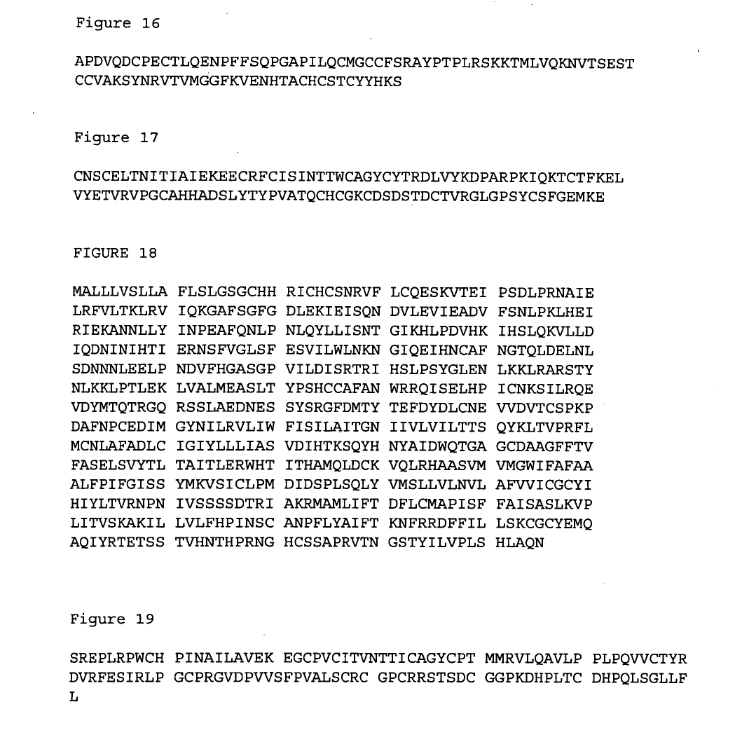

In a preferred embodiment of the invention said fusion polypeptide comprises or consists (FSH) a subunit as represented by the amino acid sequence in FIG. 16.

It is noted that the FSH α subunit is common to the hormones luteinising (LH) hormone and thyroid stimulating hormone (TSH) and therefore a claim to FSH α subunit is equivalent to a claim to LH and FSH. It is the β subunit of the respective hormones that confers receptor specificity.

In a further alternative preferred embodiment of the invention said fusion polypeptide comprises or consists of the follicle stimulating hormone (FSH) β subunit.

In a preferred embodiment of the invention said fusion polypeptide comprises or consists of the follicle stimulating hormone (FSH) β subunit as represented by the amino acid sequence in FIG. 17.

In a preferred embodiment of the invention said fusion polypeptide comprises or consists of the extracellular domain of follicle stimulating hormone receptor (FSHR).

In a preferred embodiment of the invention said fusion polypeptide comprises or consists of the extracellular domain of follicle stimulating hormone receptor (FSHR) as represented by the amino acid sequence in FIG. 18 with reference to Table 2.

In an alternative preferred embodiment of the invention said fusion polypeptide comprises or consists of the LH β subunit.

In a preferred embodiment of the invention said fusion polypeptide comprises or consists of the LHR subunit as represented in FIG. 19.

In a preferred embodiment of the invention said fusion polypeptide comprises or consists of the extracellular domain of the LH receptor.

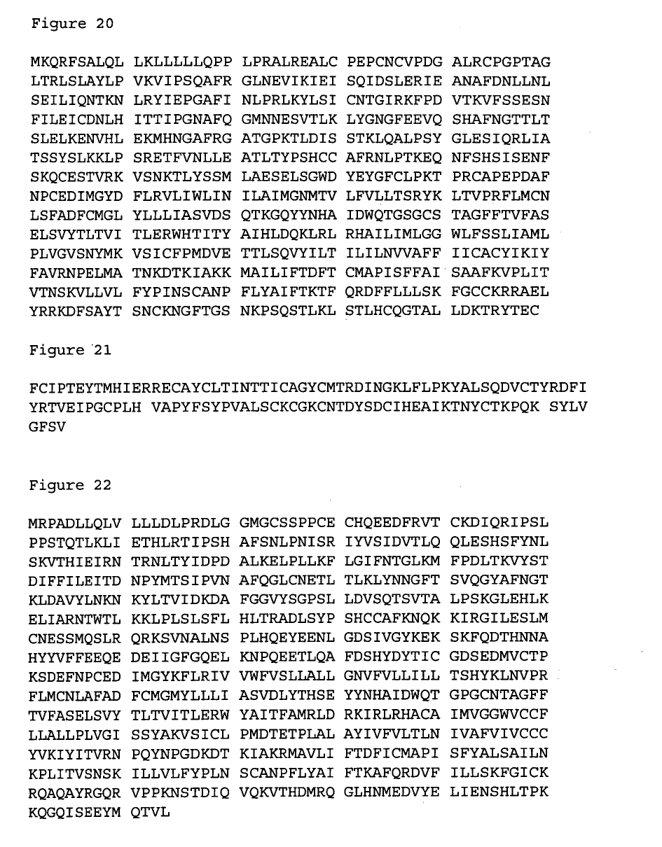

In a preferred embodiment of the invention said fusion polypeptide comprises or consists of the extracellular domain of the LH receptor as represented in FIG. 20 with reference to Table 3.

In a preferred embodiment of the invention said fusion polypeptide comprises or consists of a TSH β subunit.

In a preferred embodiment of the invention said fusion polypeptide comprises or consists of the amino acid as represented in FIG. 21.

In a further preferred embodiment of the invention said fusion polypeptide comprises of the extracellular domain of the TSH receptor.

In a preferred embodiment of the invention said fusion polypeptide comprises or consists of the extracellular domain of the TSH receptor as represented in FIG. 22 with reference to Table 4.

In an alternative preferred embodiment of the invention said first and second polypeptides are ligand binding domains that bind receptor polypeptides wherein said domains are linked in tandem.

In a further preferred embodiment of the invention the binding domains of the polypeptide are the same or similar to each other.

In an alternative preferred embodiment of the invention said ligand binding domains are dissimilar.

In a further preferred embodiment of the invention at least one of the domains comprises a growth hormone binding domain.

In a preferred embodiment of the invention said fusion polypeptide comprises first and second growth hormone polypeptides.

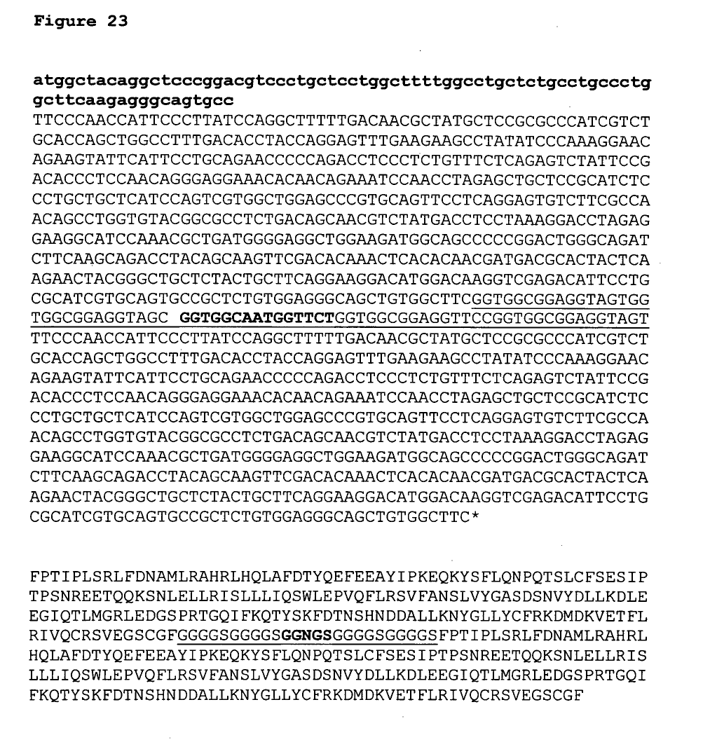

In a preferred embodiment of the invention said fusion polypeptide comprises an amino acid sequence as represented in FIG. 23, 24, 25 or 26.

In a further preferred embodiment of the invention said polypeptide comprises at least two binding domains of growth hormone, or a growth hormone variant.

In an alternative preferred embodiment of the invention said polypeptide comprises at least two binding domains of prolactin, or a prolactin variant.

In a preferred embodiment of the invention said prolactin variant polypeptide comprises an amino acid sequence wherein said amino acid sequence is modified at position 129 of human prolactin.

In a preferred embodiment of the invention said modification is an amino acid substitution. Preferably said substitution replaces a glycine amino acid residue with an arginine amino acid residue. Preferably said modification further comprises the deletion of at least 9, 10, 11, 12, 13 or 14 amino terminal amino acid residues.

In an alternative embodiment of the invention the binding domains of the polypeptide are dissimilar to each other.

In a preferred embodiment of the invention said polypeptide comprises a first binding domain that is a growth hormone binding domain and a second binding domain that is a prolactin binding domain.

Preferably said polypeptide consists of a growth hormone binding domain and a prolactin binding domain.

In an alternative preferred embodiment of the invention said polypeptide comprises a first binding domain that is a modified growth hormone binding domain and a second binding domain that is a modified prolactin binding domain.

Preferably said polypeptide consists of a modified growth hormone binding domain and a modified prolactin binding domain.

In a preferred embodiment of the invention said modified growth hormone binding domain comprises an amino acid susbstitution at amino acid position glycine 120.

Preferably, said modification is a substitution of glycine 120 for an amino acid selected from the group consisting of arginine, lysine, tryptophan, tyrosine, phenylalanine, or glutamic acid.

In a preferred embodiment of the invention said modification is the substitution of glycine 120 with an arginine amino acid residue.

In a further preferred embodiment of the invention said modified prolactin binding domain comprises a modification of glycine 129. Preferably said modification is the substitution of glycine 129 with an arginine amino acid residue. Preferably said modification further comprises the deletion of at least 9, 10, 11, 12, 13 or 14 amino terminal amino acid residues.

In an alternative preferred embodiment of the invention said first and second polypeptides comprise antibody variable region binding domains.

In a preferred embodiment of the invention said antibody variable region binding domains bind the same or a similar epitope.

In an alternative preferred embodiment of the invention said antibody variable region binding domains bind dissimilar epitopes and are bivalent.

Various fragments of immunoglobulin or antibodies are known in the art, i.e., Fab, Fab2, F(ab′)2, Fv, Fc, Fd, scFvs, etc. A Fab fragment is a multimeric protein consisting of the immunologically active portions of an immunoglobulin heavy chain variable region and an immunoglobulin light chain variable region, covalently coupled together and capable of specifically binding to an antigen. Fab fragments are generated via proteolytic cleavage (with, for example, papain) of an intact immunoglobulin molecule. A Fab2 fragment comprises two joined Fab fragments. When these two fragments are joined by the immunoglobulin hinge region, a F(ab′)2 fragment results. An Fv fragment is multimeric protein consisting of the immunologically active portions of an immunoglobulin heavy chain variable region and an immunoglobulin light chain variable region covalently coupled together and capable of specifically binding to an antigen. A fragment could also be a single chain polypeptide containing only one light chain variable region, or a fragment thereof that contains the three CDRs of the light chain variable region, without an associated heavy chain variable region, or a fragment thereof containing the three CDRs of the heavy chain variable region, without an associated light chain moiety; and multi specific antibodies formed from antibody fragments, this has for example been described in U.S. Pat. No 6,248,516. Fv fragments or single region (domain) fragments are typically generated by expression in host cell lines of the relevant identified regions. These and other immunoglobulin or antibody fragments are within the scope of the invention and are described in standard immunology textbooks such as Paul, Fundamental Immunology or Janeway et al. Immunobiology (cited above). Molecular biology now allows direct synthesis (via expression in cells or chemically) of these fragments, as well as synthesis of combinations thereof.

It is possible to create single variable regions, so called single chain antibody variable region fragments (scFvs). If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's. Alternatively said fragments are “domain antibody fragments”. Domain antibodies are the smallest binding part of an antibody (approximately 13 kDa). Examples of this technology is disclosed in U.S. Pat. No. 6,248, 516, U.S. Pat. No. 6,291,158, U.S. Pat. No. 6,127,197 and EP0368684 which are all incorporated by reference in their entirety.

In a preferred embodiment of the invention said antibody fragment is a single chain antibody variable region fragment.

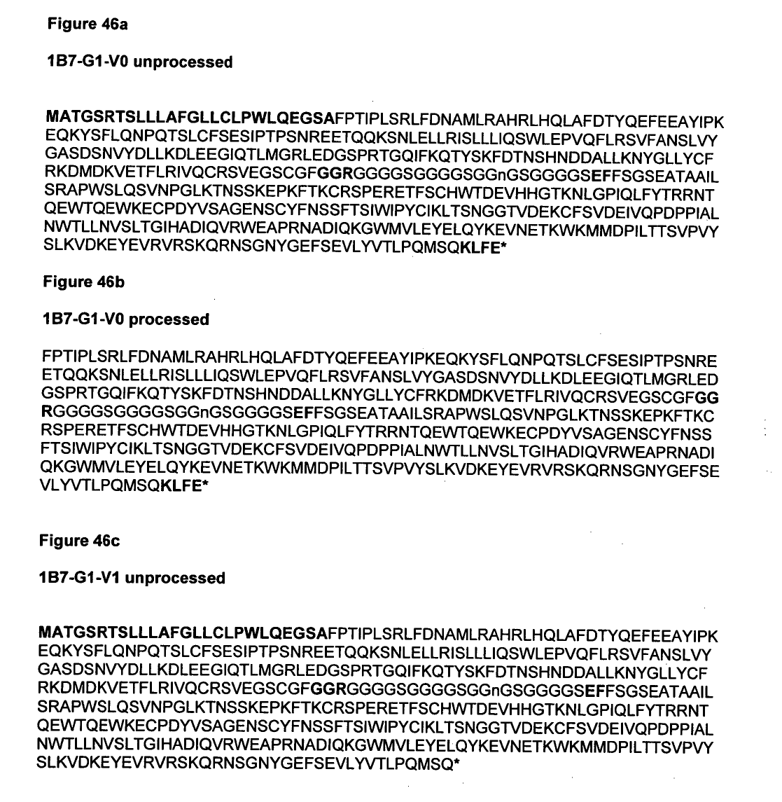

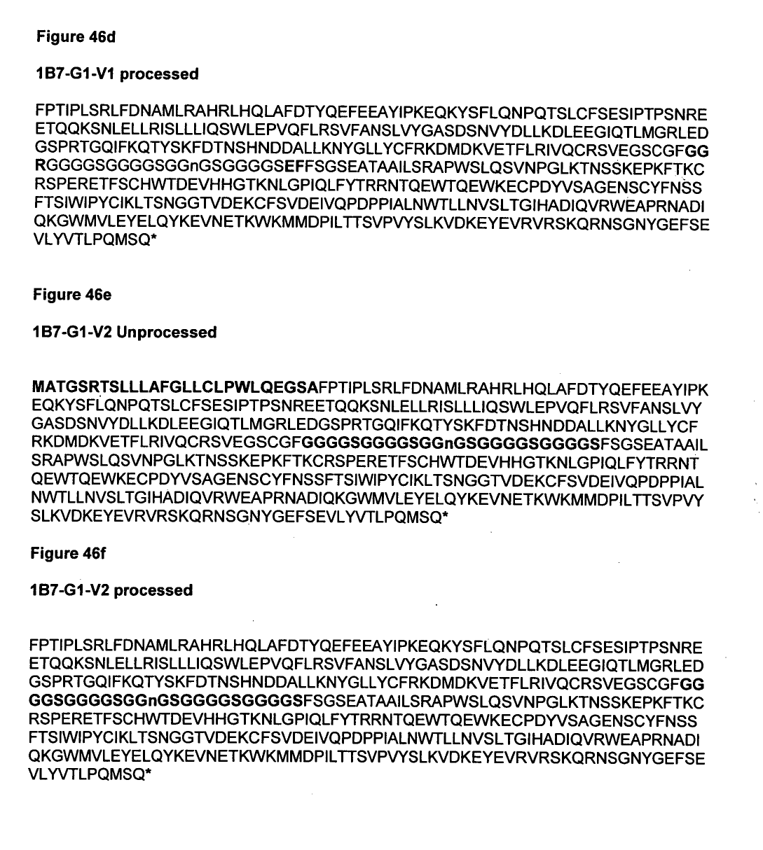

In a preferred embodiment of the invention said polypeptide comprises or consists of an amino acid sequence selected from the group consisting of the sequences represented in FIG. 46a, 46b, 46c, 46d, 46e or 46f.

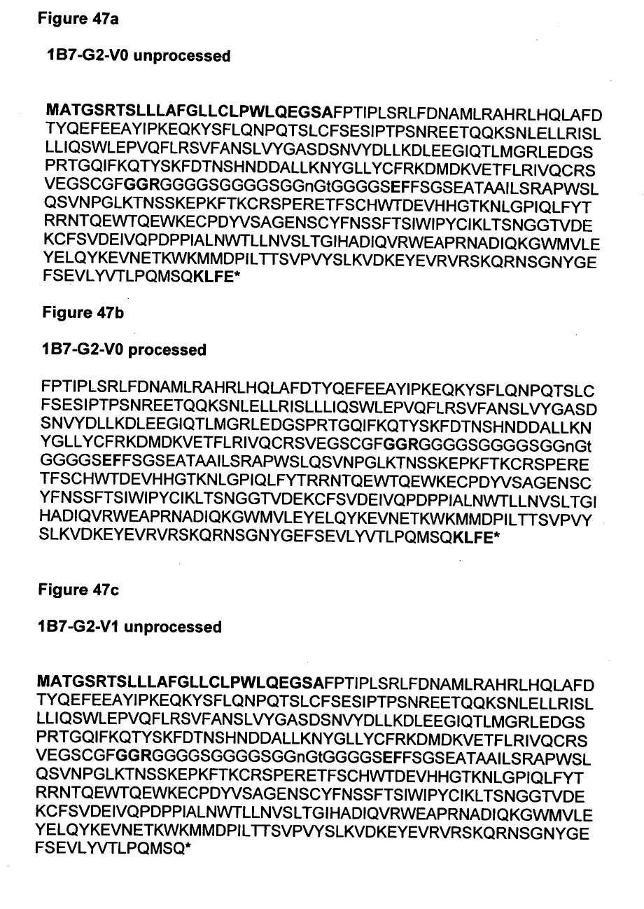

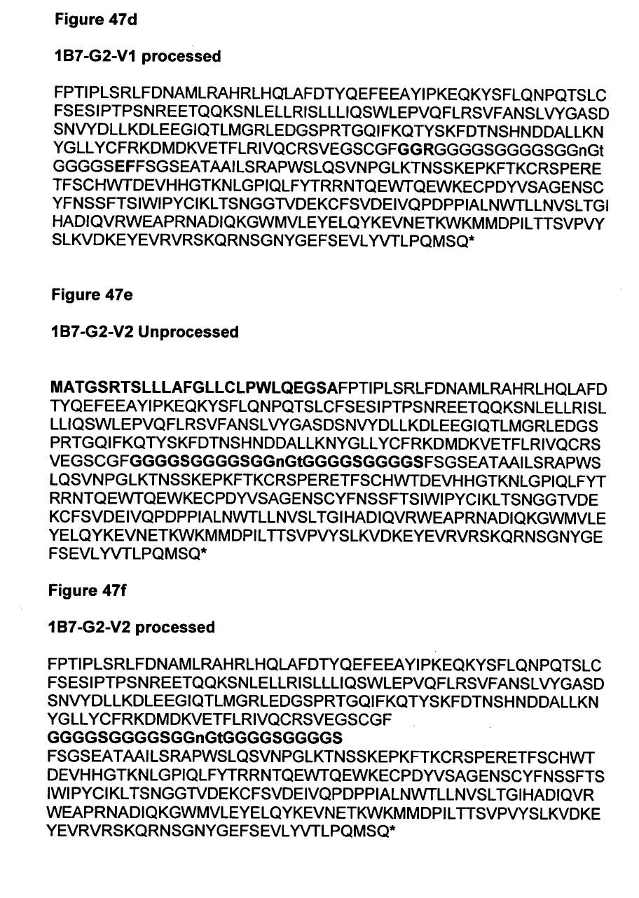

In a preferred embodiment of the invention said polypeptide comprises or consists of an amino acid sequence selected from the group consisting of the sequences represented in FIG. 47a, 47b, 47c, 47d, 47e or 47f.

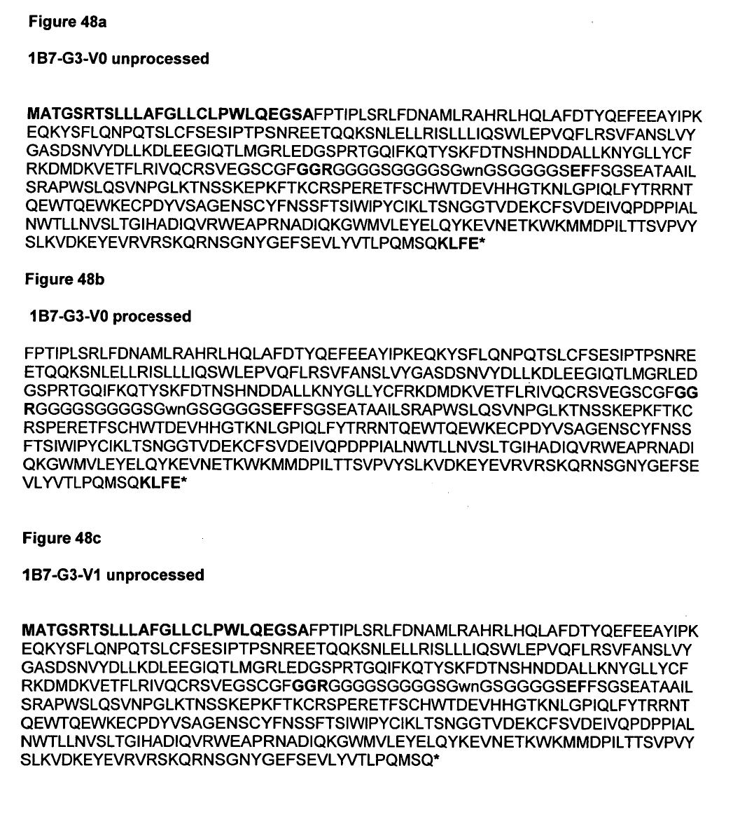

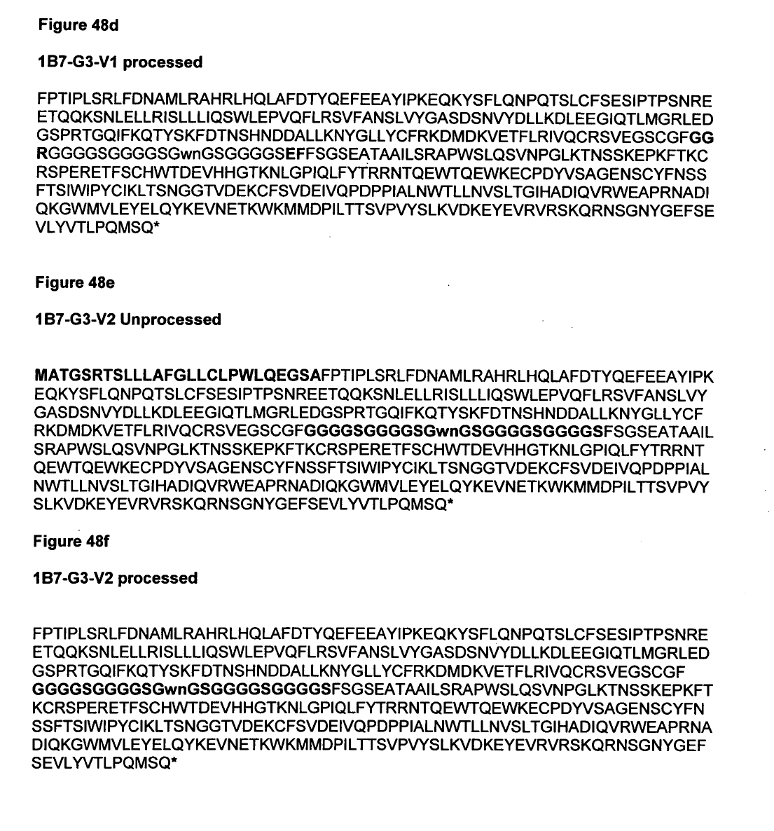

In a preferred embodiment of the invention said polypeptide comprises or consists of an amino acid sequence selected from the group consisting of the sequences represented in FIG. 48a, 48b, 48c, 48d, 48e or 48f.

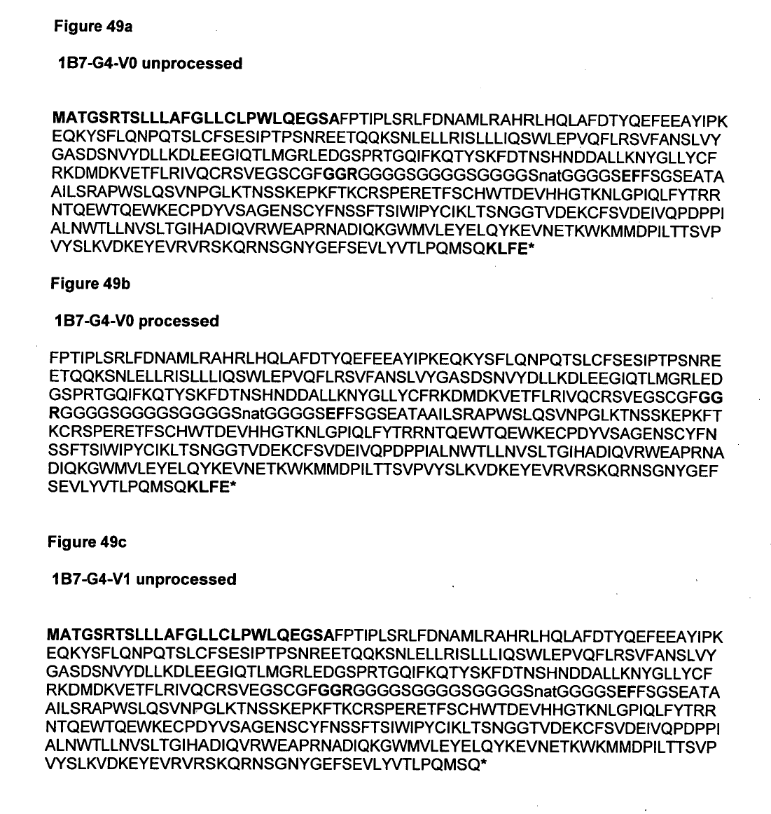

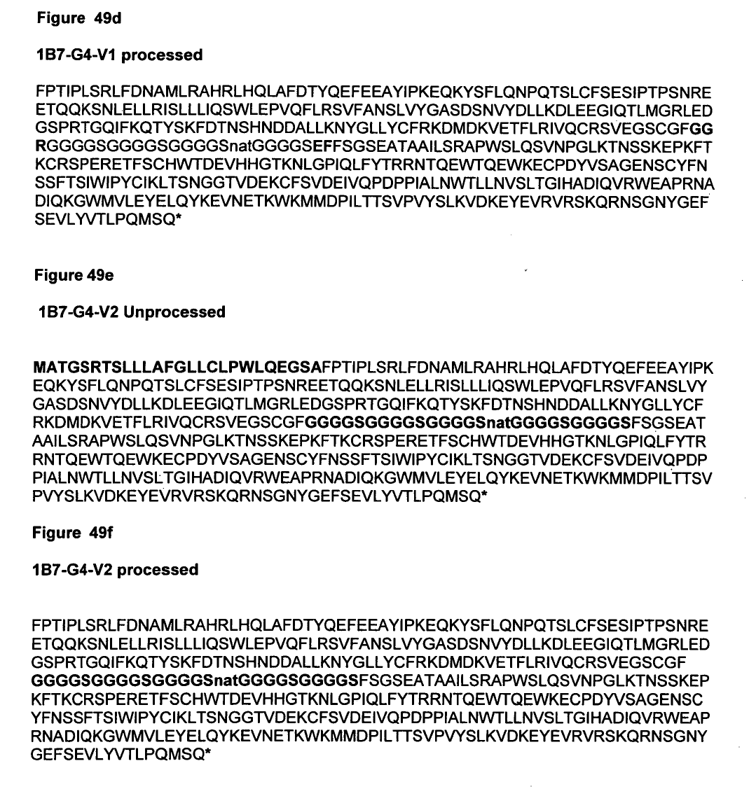

In a preferred embodiment of the invention said polypeptide comprises or consists of an amino acid sequence selected from the group consisting of the sequences represented in FIG. 49a, 49b, 49c, 49d, 49e or 49f.

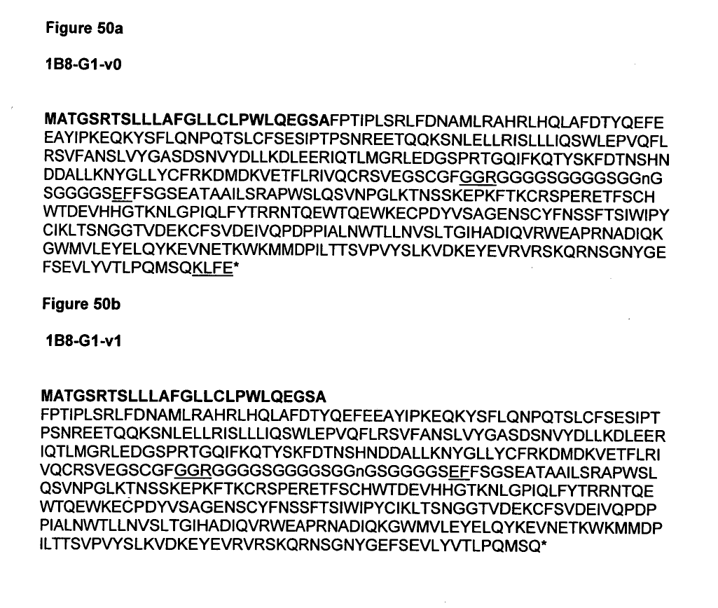

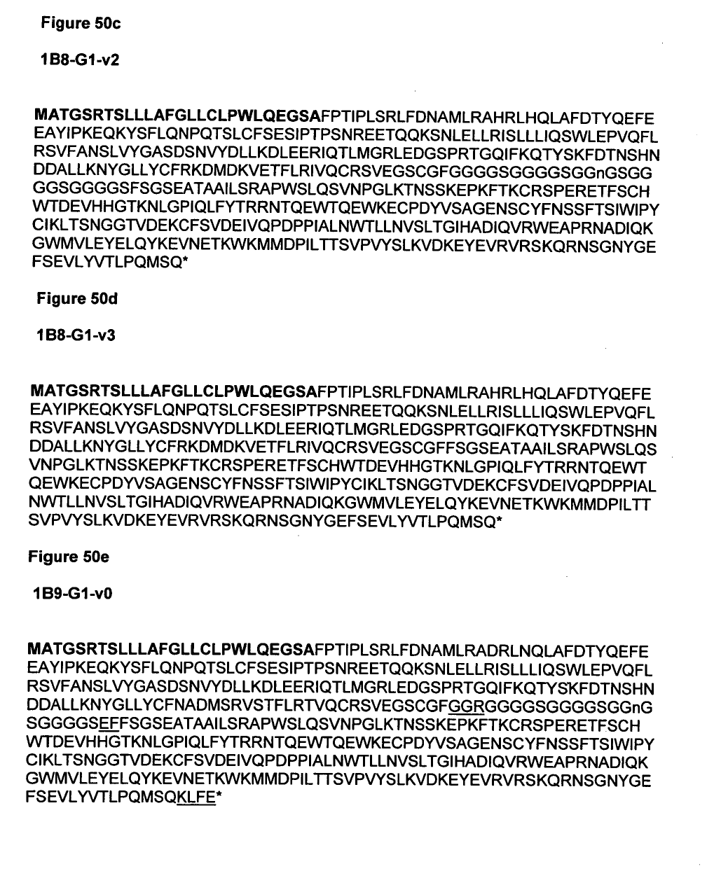

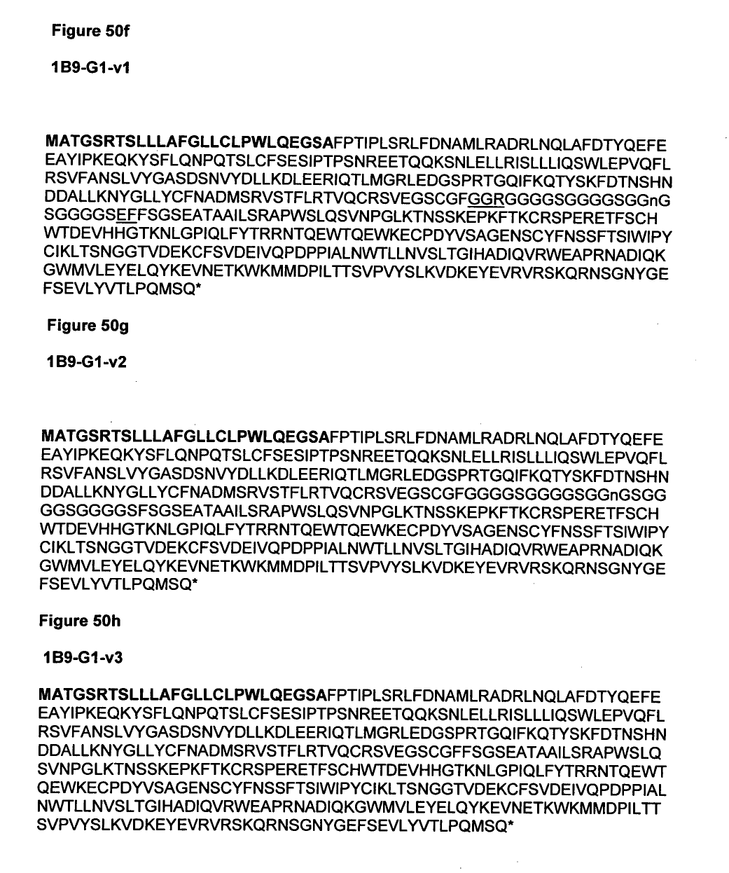

In a preferred embodiment of the invention said polypeptide comprises or consists of an amino acid sequence selected from the group consisting of the sequences represented in FIG. 50a, 50b, 50c, 50d, 50e, 50f or 50h.

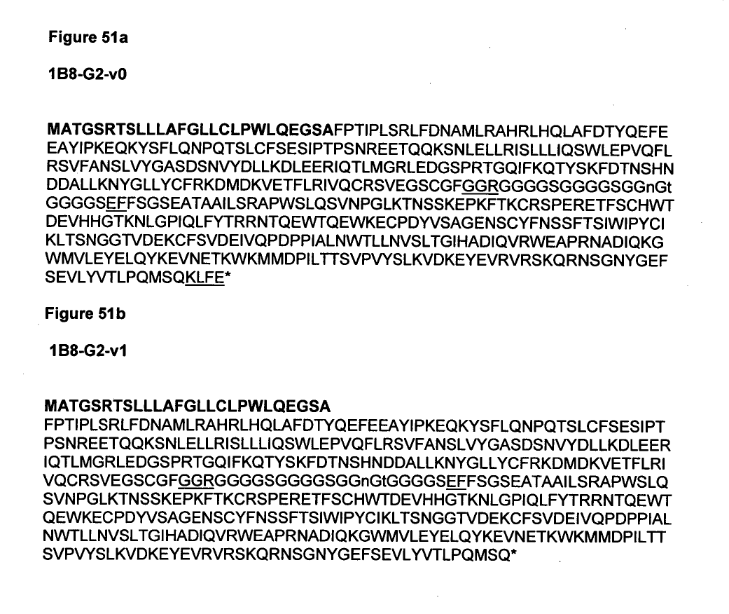

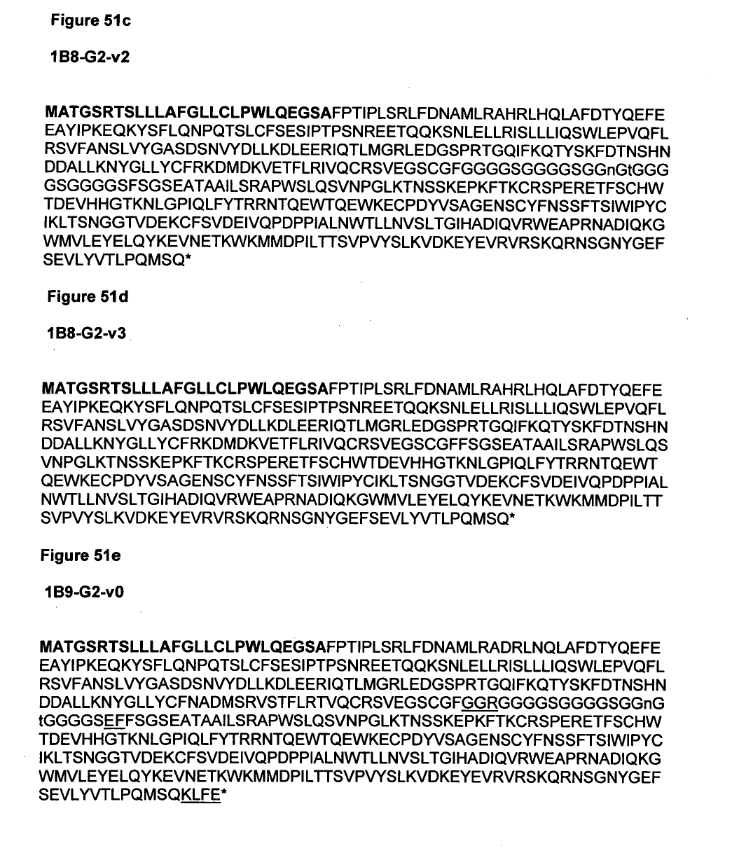

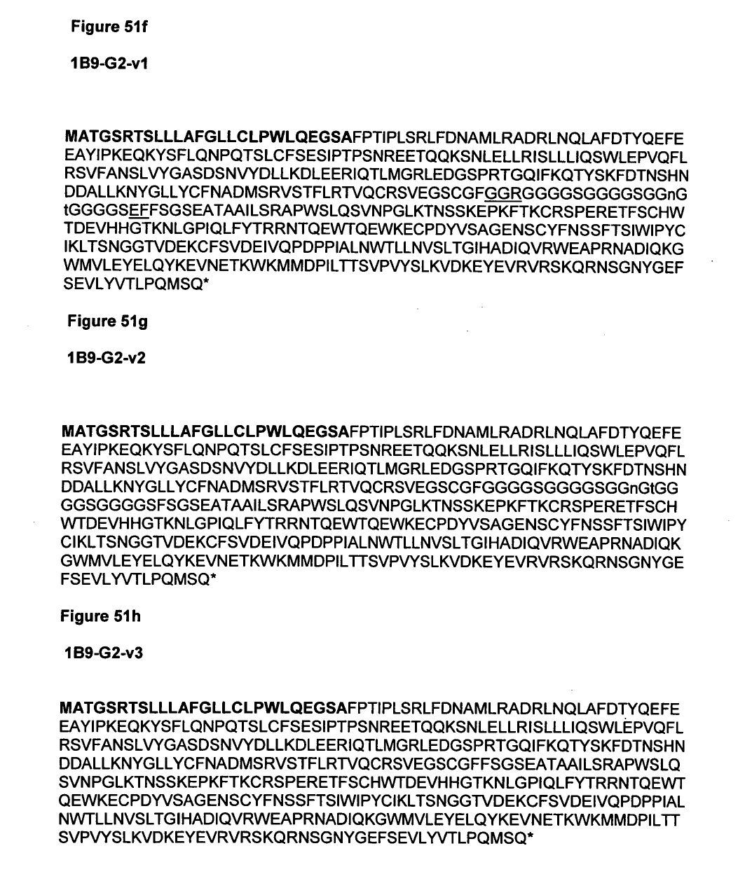

In a preferred embodiment of the invention said polypeptide comprises or consists of an amino acid sequence selected from the group consisting of the sequences represented in FIG. 51a, 51b, 51c, 51d, 51e, 51f or 51h.

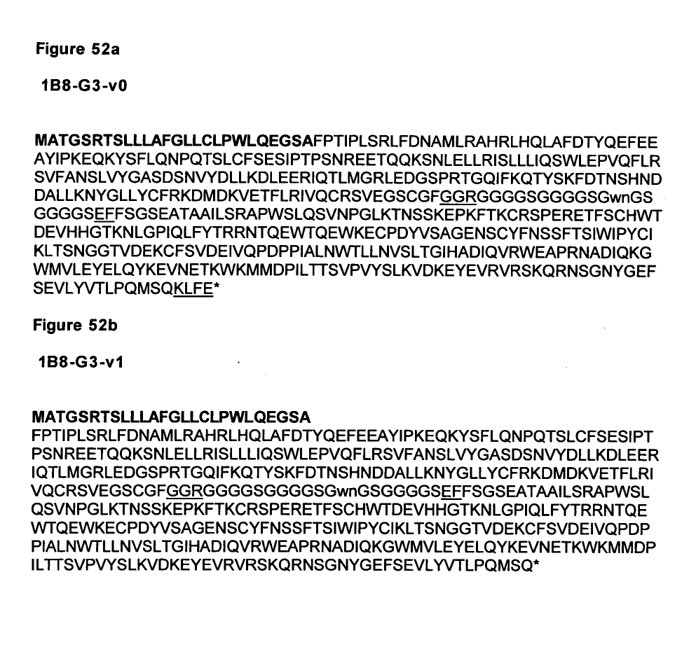

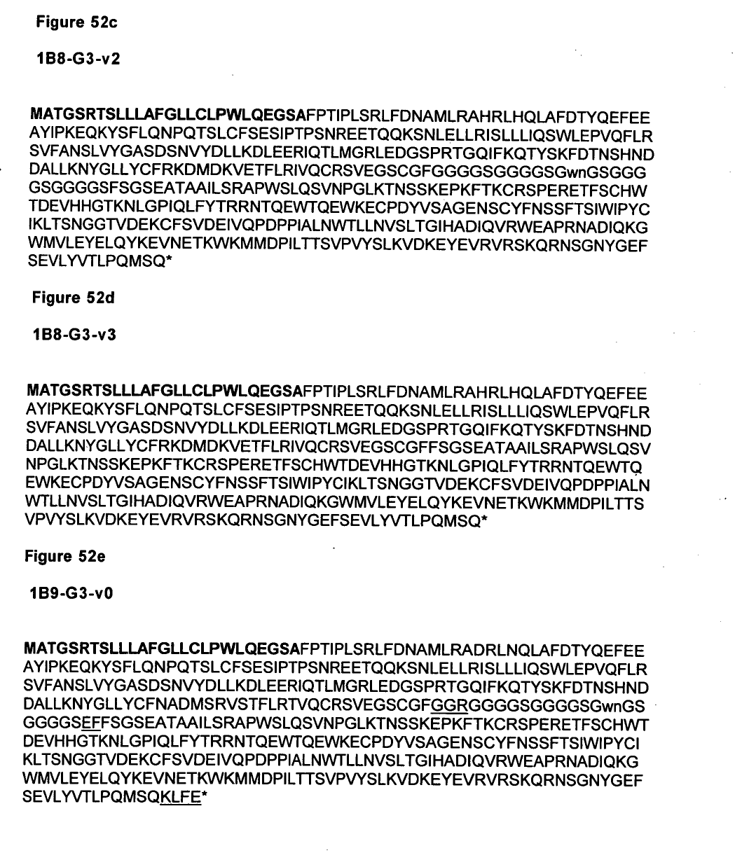

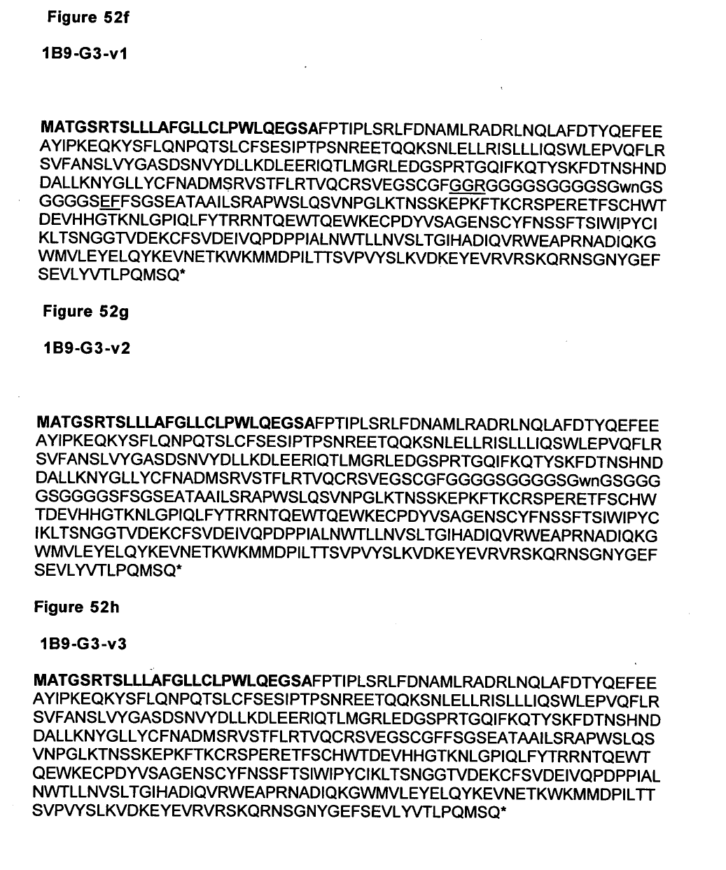

In a preferred embodiment of the invention said polypeptide comprises or consists of an amino acid sequence selected from the group consisting of the sequences represented in FIG. 52a, 52b, 52c, 52d, 52e, 52f or 52h.

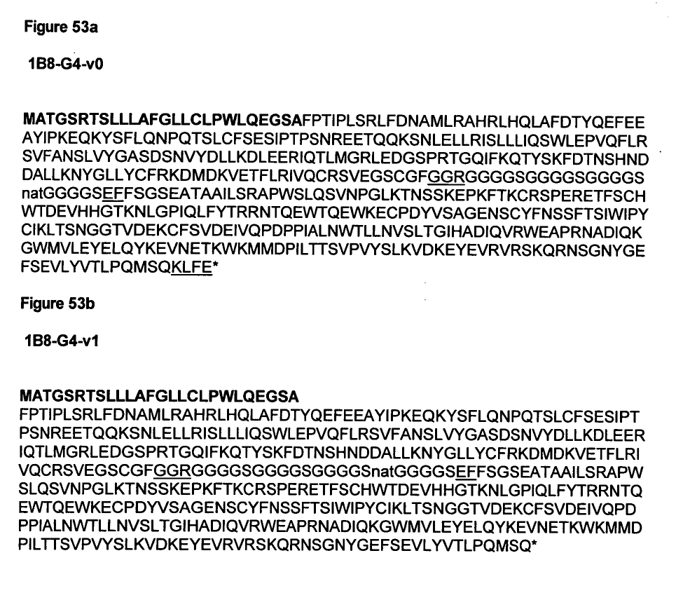

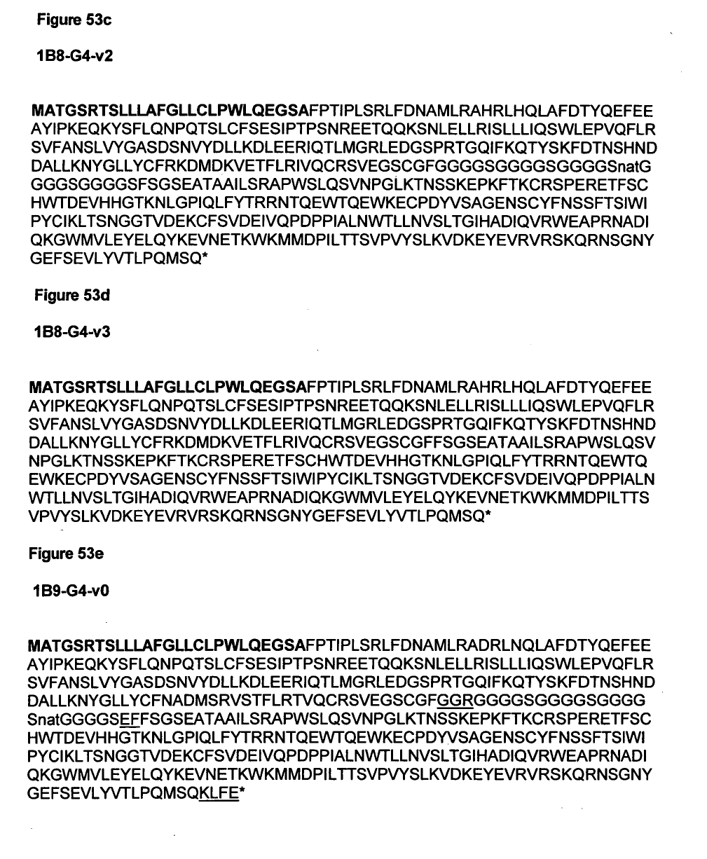

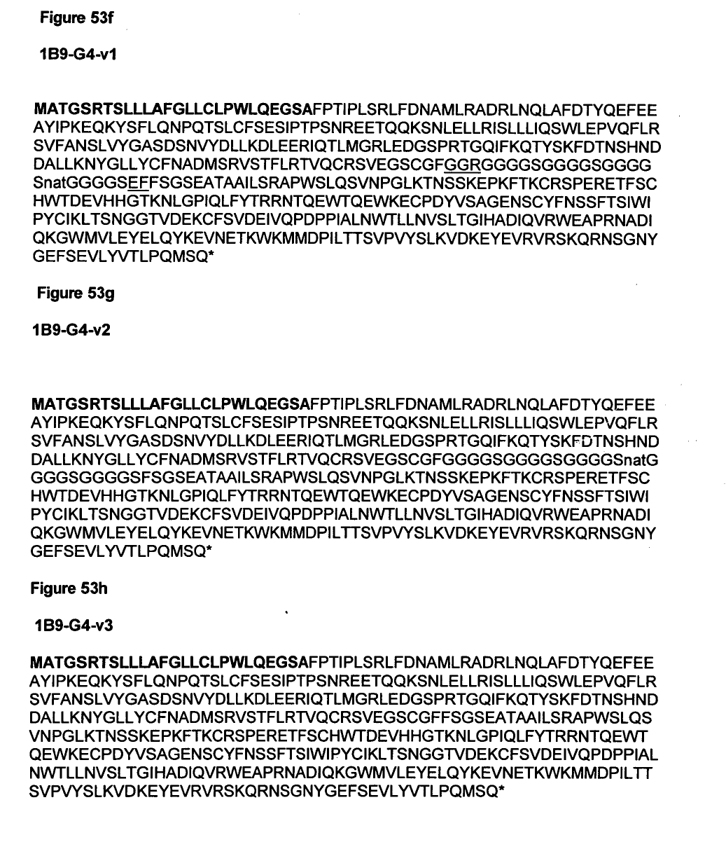

In a preferred embodiment of the invention said polypeptide comprises or consists of an amino acid sequence selected from the group consisting of the sequences represented in FIGS. 53a, 53b, 53c, 53d, 53e, 53f or 53h.

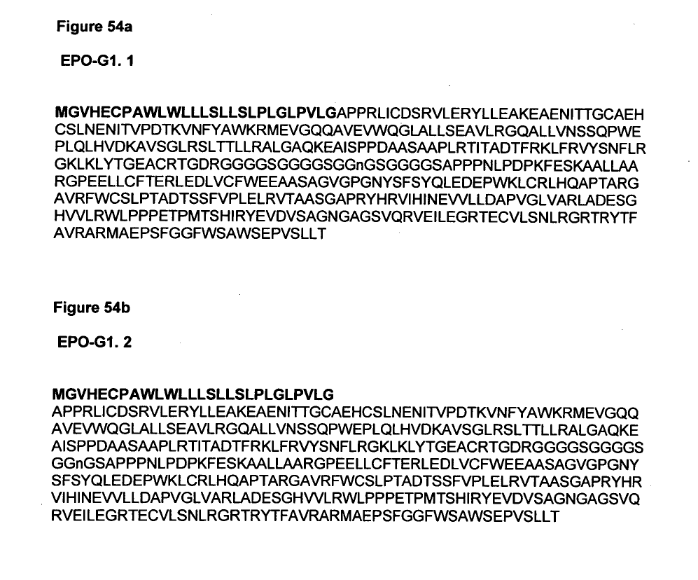

In a preferred embodiment of the invention said polypeptide comprises or consists of an amino acid sequence selected from the group consisting of the sequences represented in FIG. 54a or 54b.

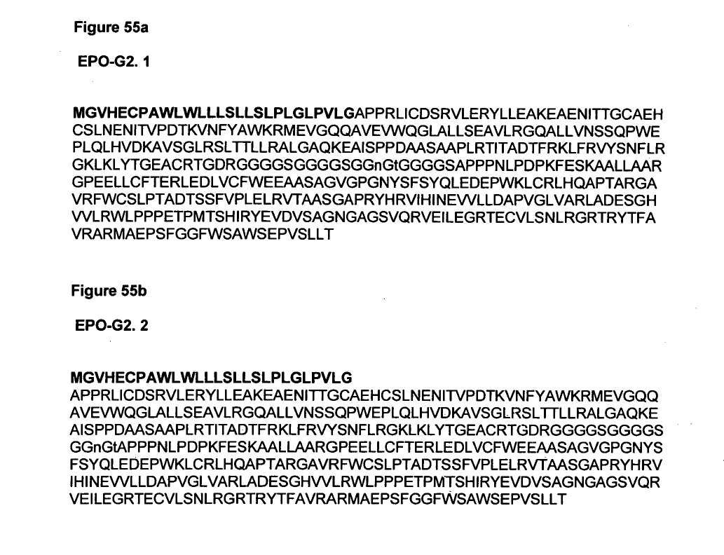

In a preferred embodiment of the invention said polypeptide comprises or consists of an amino acid sequence selected from the group consisting of the sequences represented in FIG. 55a or 55b.

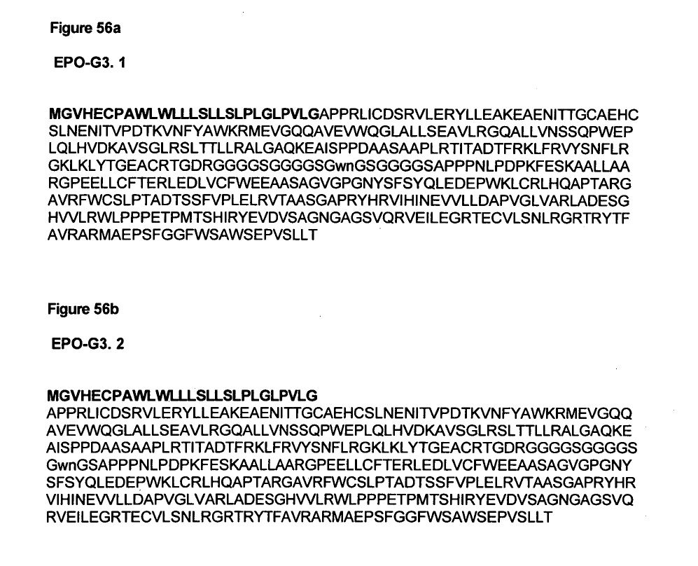

In a preferred embodiment of the invention said polypeptide comprises or consists of an amino acid sequence selected from the group consisting of the sequences represented in FIG. 56a or 56b

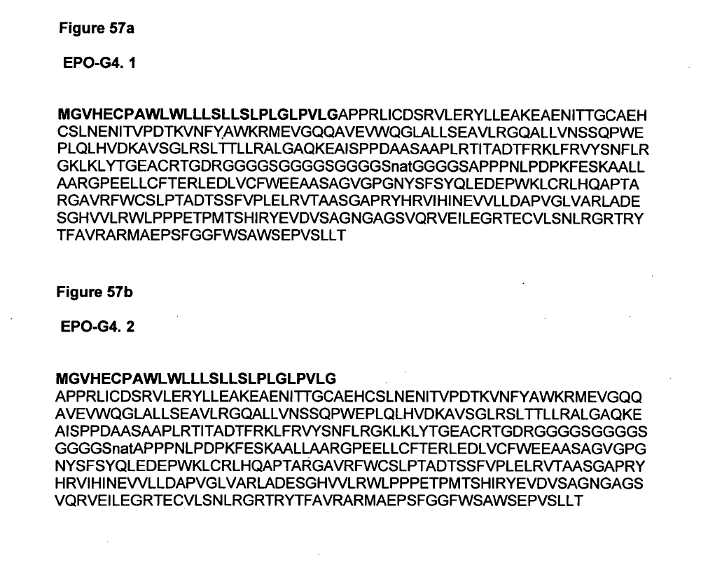

In a preferred embodiment of the invention said polypeptide comprises or consists of an amino acid sequence selected from the group consisting of the sequences represented in FIG. 57a or 57b.

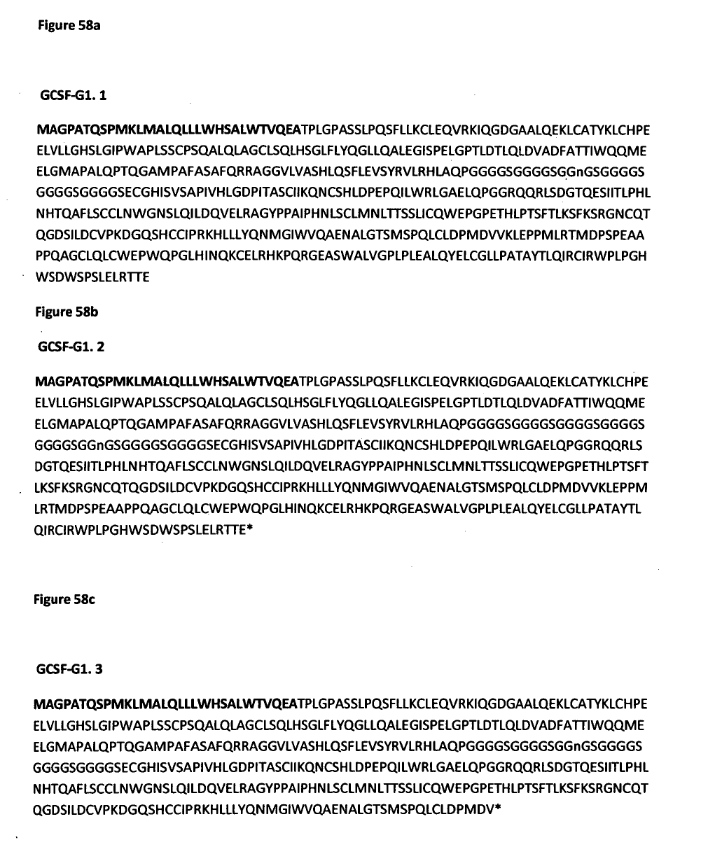

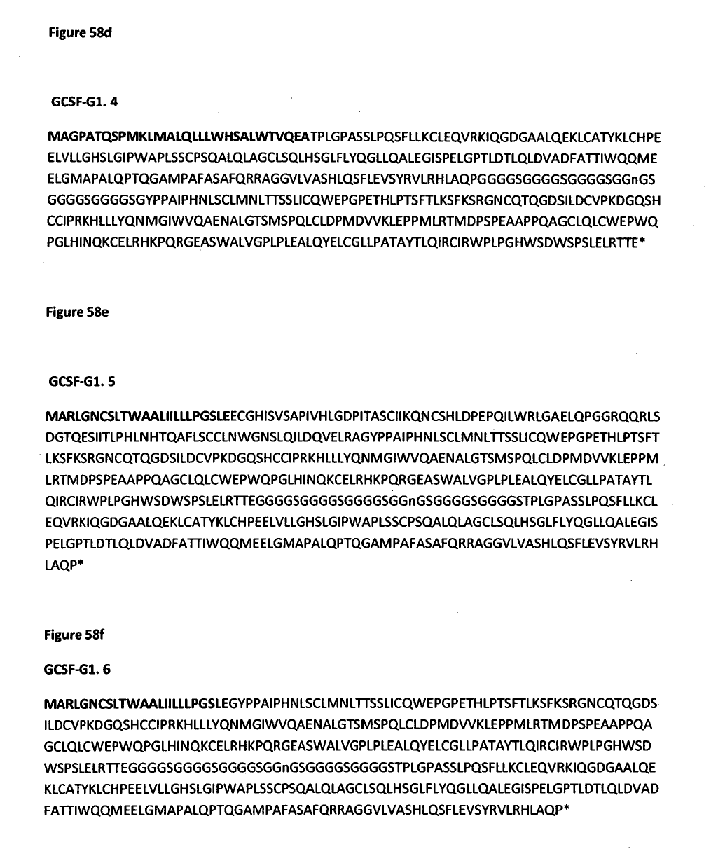

In a preferred embodiment of the invention said polypeptide comprises or consists of an amino acid sequence selected from the group consisting of the sequences represented in FIG. 58a, 58b, 58c, 58d, 58e or 58f.

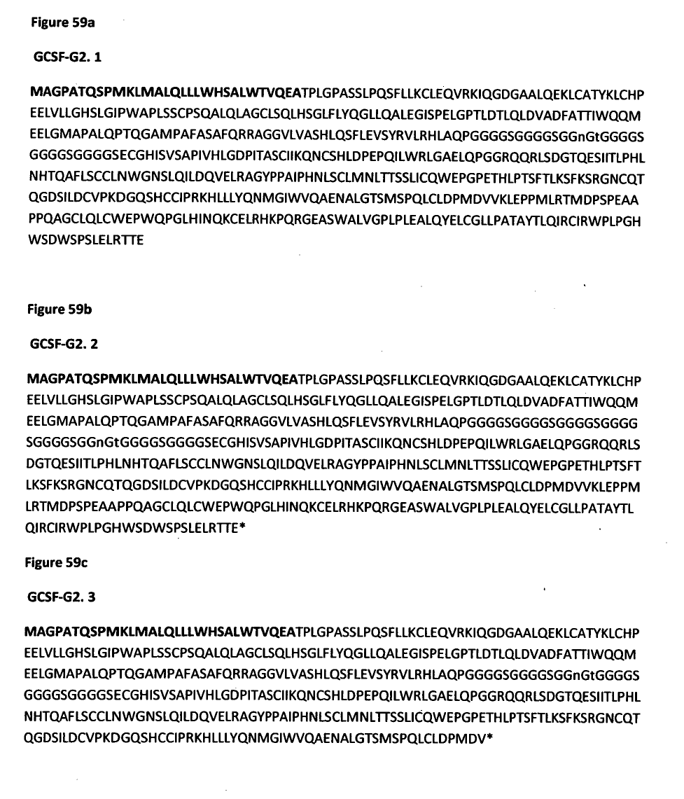

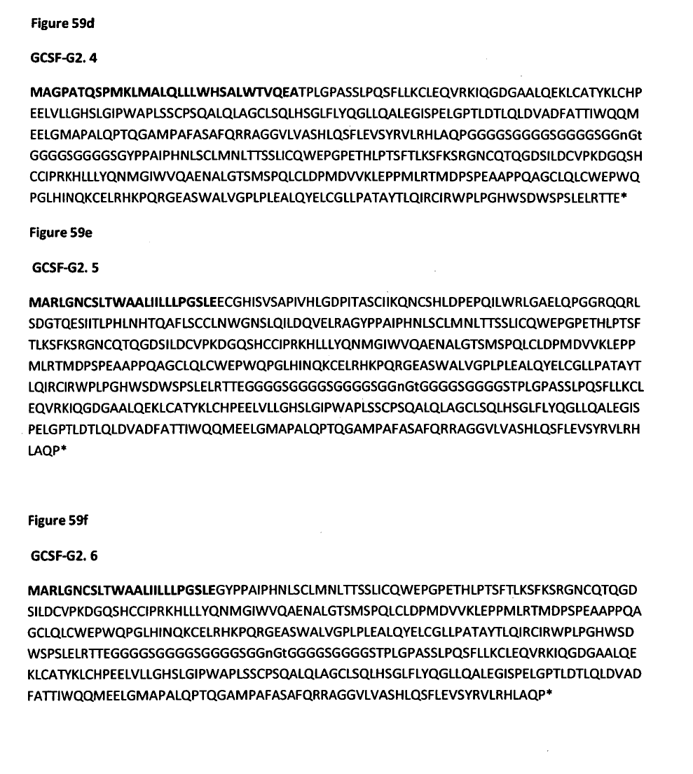

In a preferred embodiment of the invention said polypeptide comprises or consists of an amino acid sequence selected from the group consisting of the sequences represented in FIG. 59a, 59a, 59b, 59c, 59d, 59e or 59f.

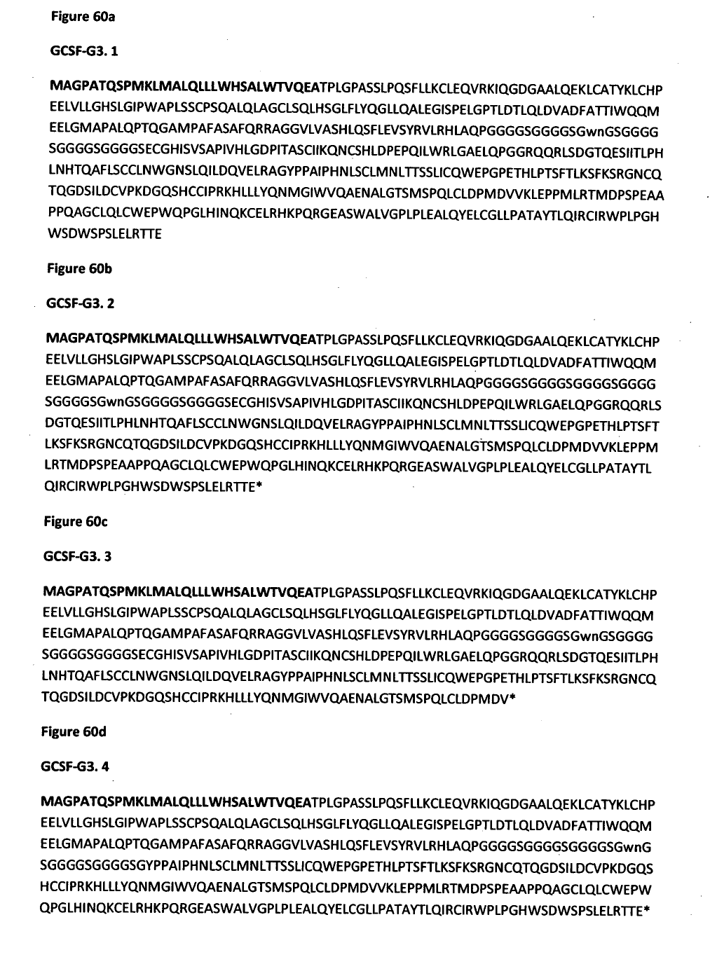

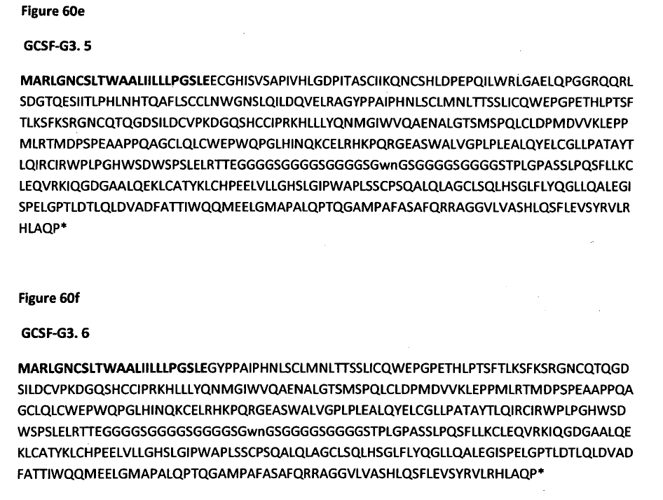

In a preferred embodiment of the invention said polypeptide comprises or consists of an amino acid sequence selected from the group consisting of the sequences represented in FIG. 60a, 60b, 60c, 60d, 60e or 60f.

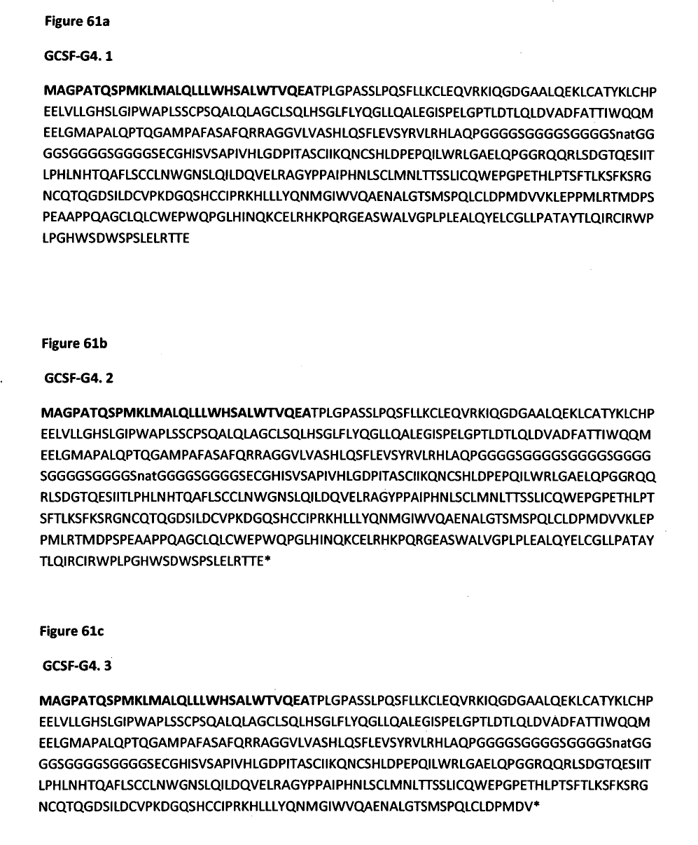

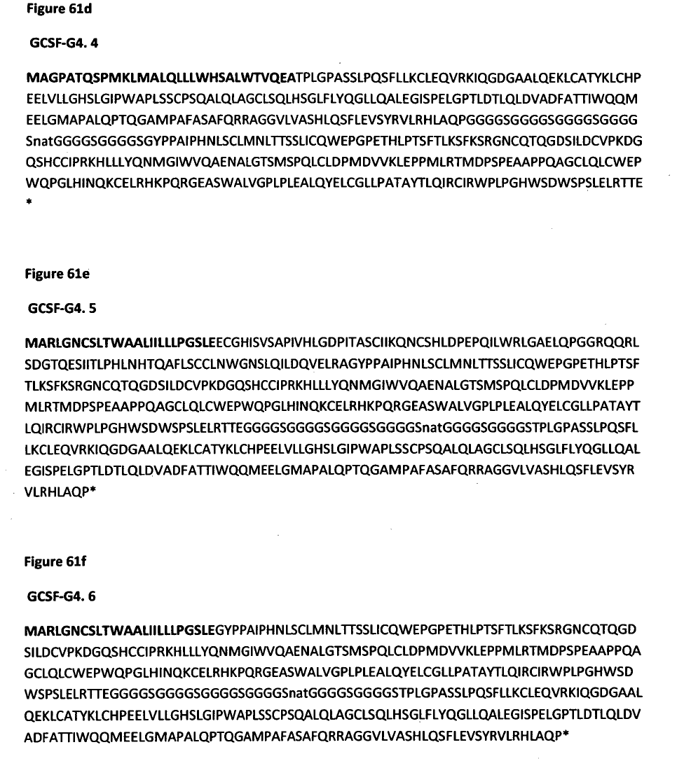

In a preferred embodiment of the invention said polypeptide comprises or consists of an amino acid sequence selected from the group consisting of the sequences represented in FIG. 61a, 61b, 61c, 61d, 61e or 61f.

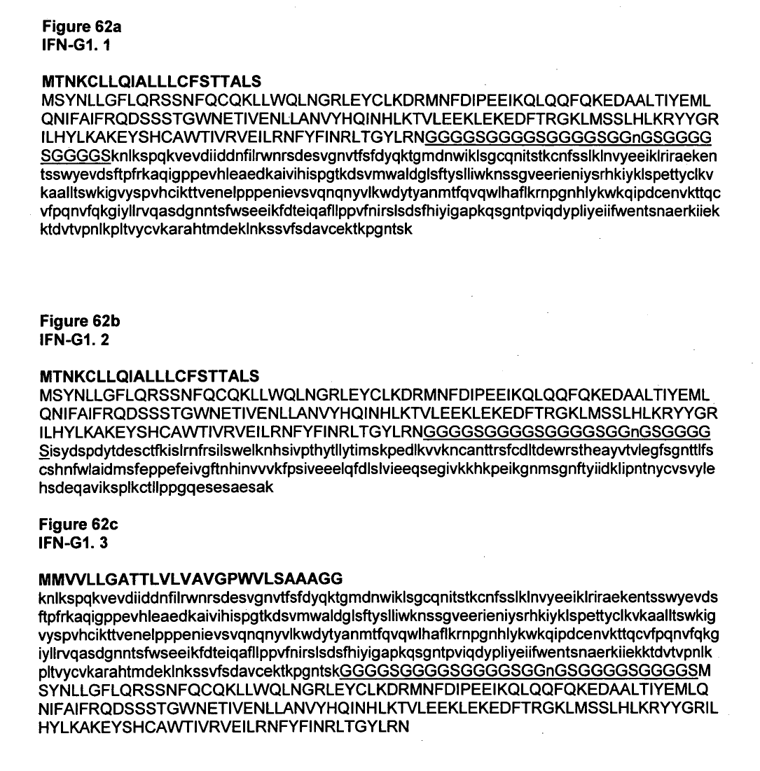

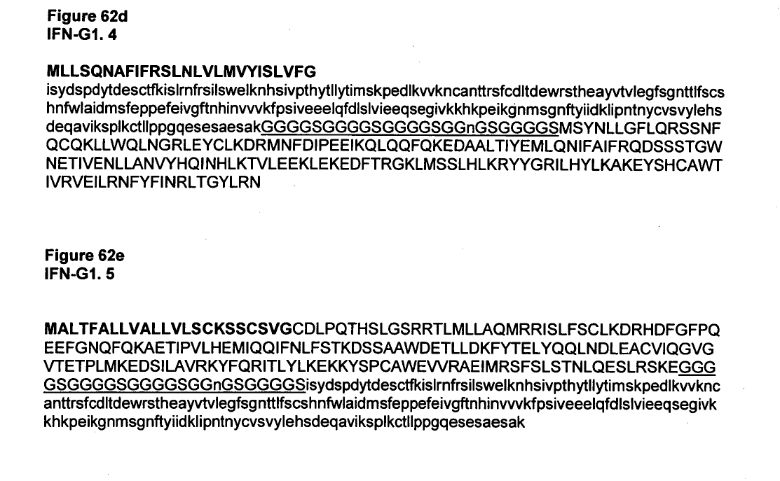

In a preferred embodiment of the invention said polypeptide comprises or consists of an amino acid sequence selected from the group consisting of the sequences represented in FIG. 62a, 62b, 62c, 62d or 62e.

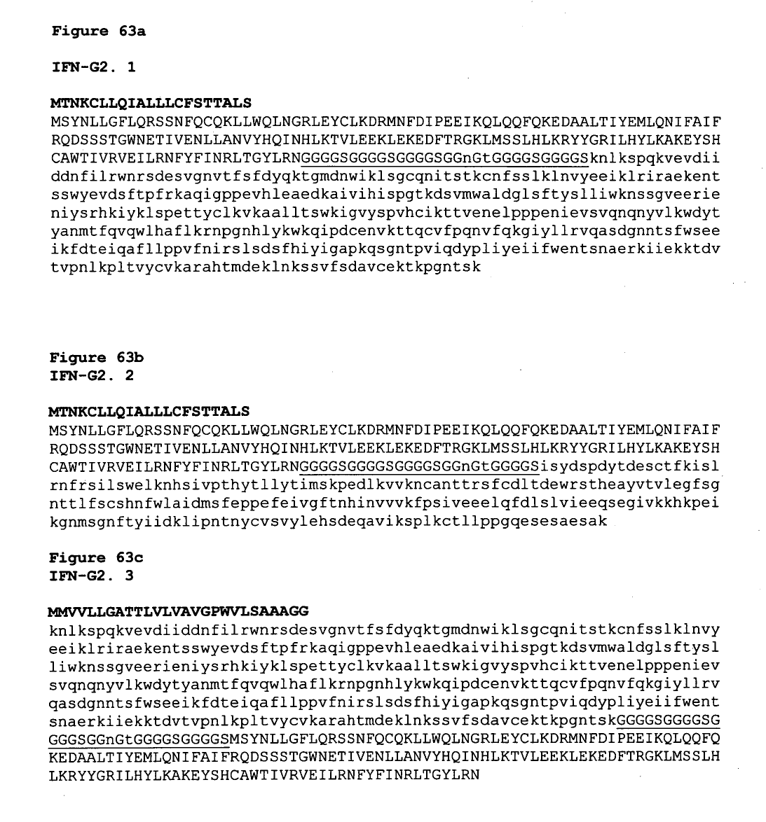

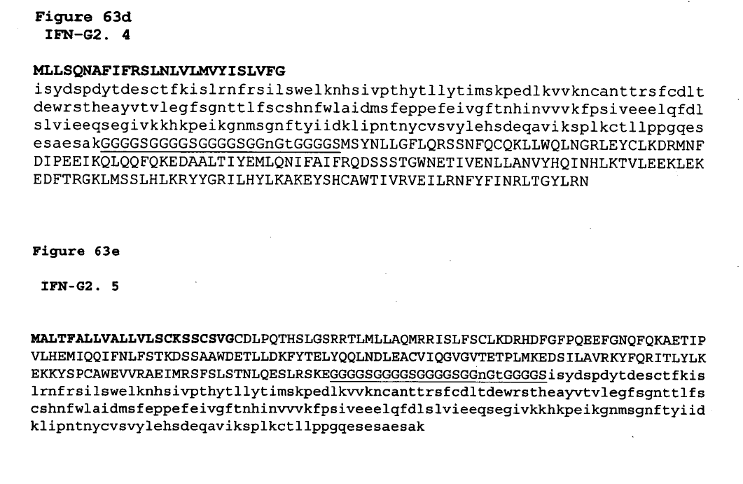

In a preferred embodiment of the invention said polypeptide comprises or consists of an amino acid sequence selected from the group consisting of the sequences represented in FIG. 63a, 63b, 63c, 63d or 63e.

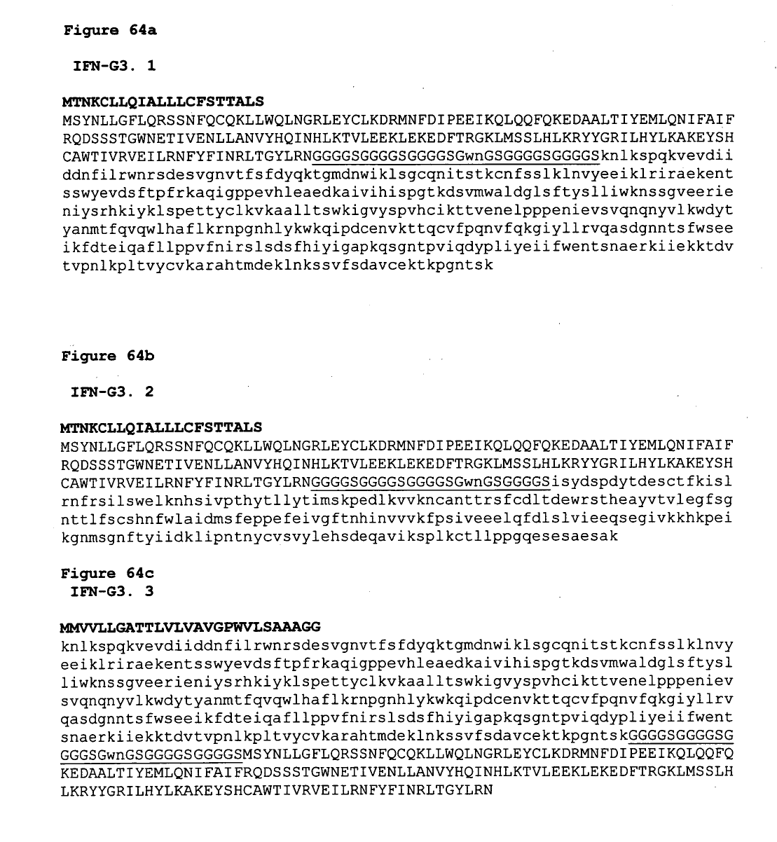

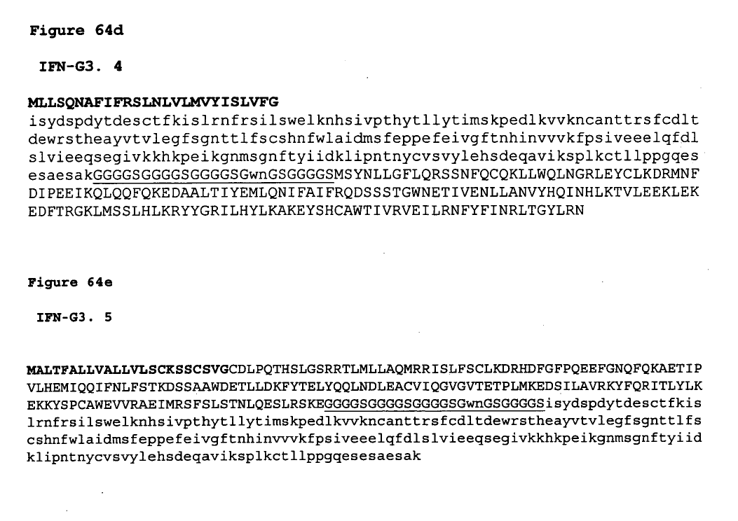

In a preferred embodiment of the invention said polypeptide comprises or consists of an amino acid sequence selected from the group consisting of the sequences represented in FIG. 64a, 64b, 64c, 64d or 64e.

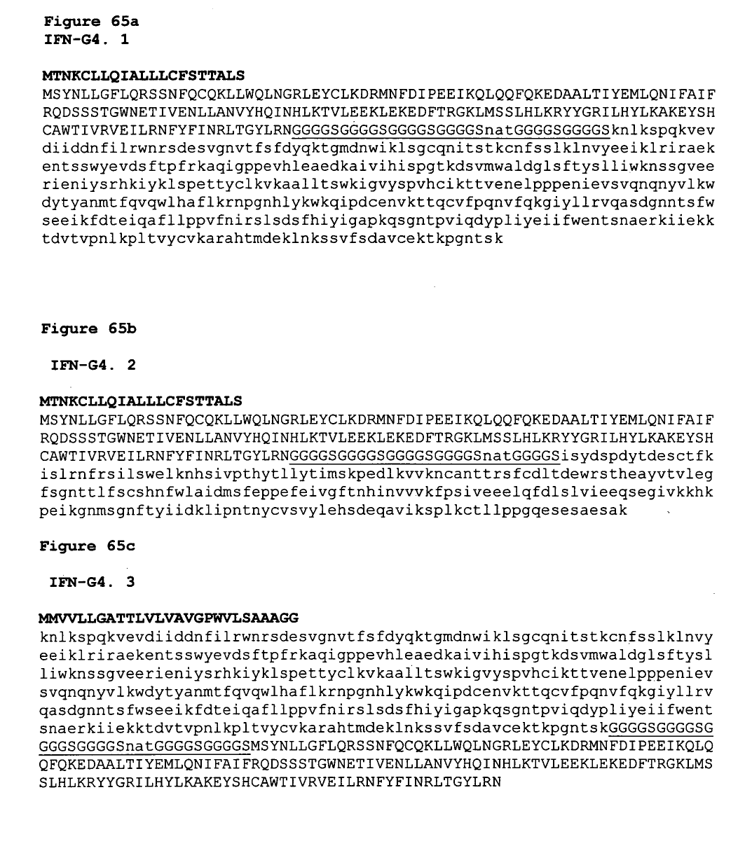

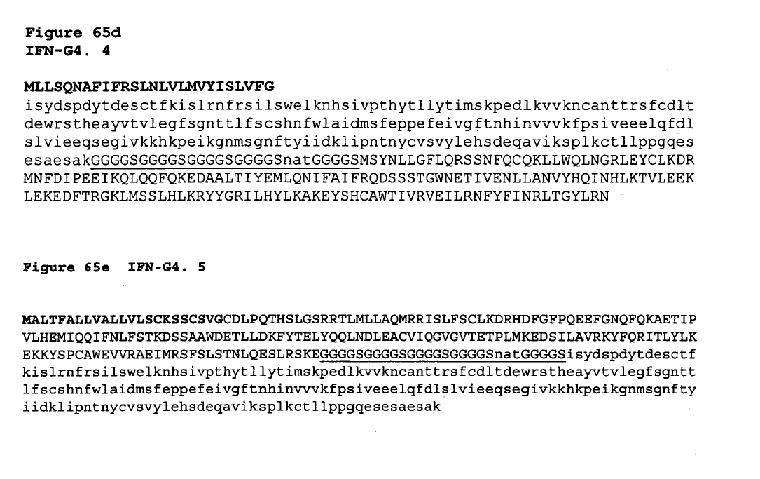

In a preferred embodiment of the invention said polypeptide comprises or consists of an amino acid sequence selected from the group consisting of the sequences represented in FIG. 65a, 65b, 65c, 65d or 65e.

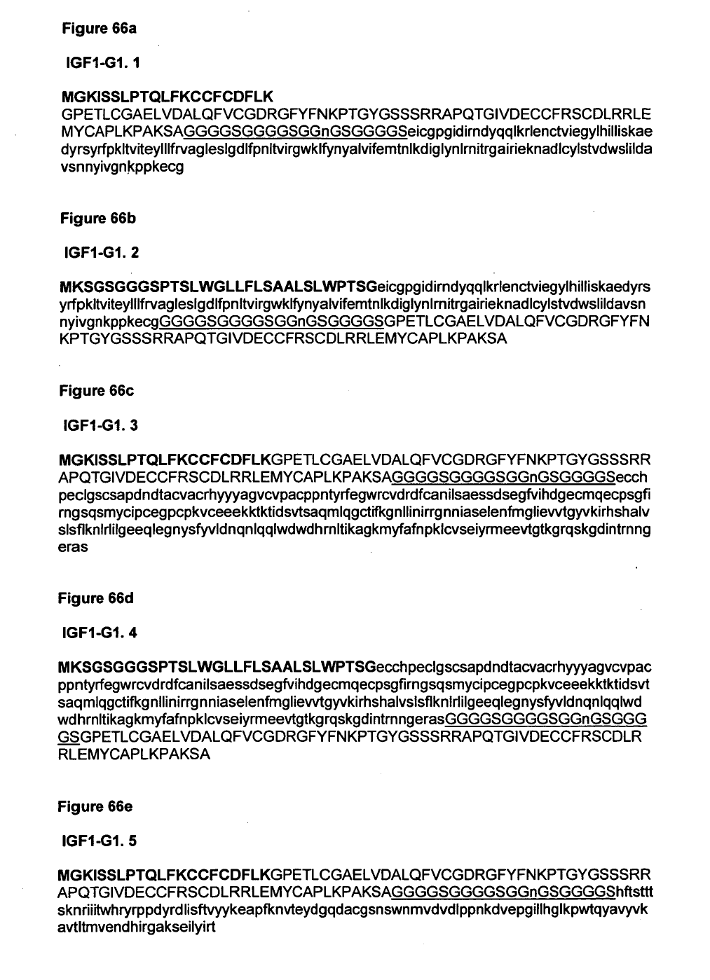

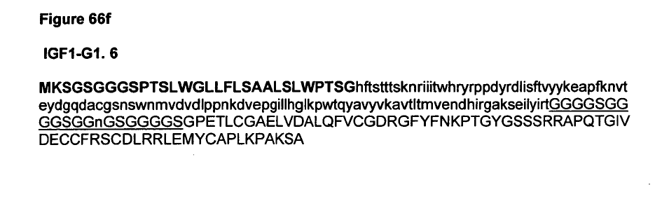

In a preferred embodiment of the invention said polypeptide comprises or consists of an amino acid sequence selected from the group consisting of the sequences represented in FIG. 66a, 66b, 66c, 66d, 66e or 66f.

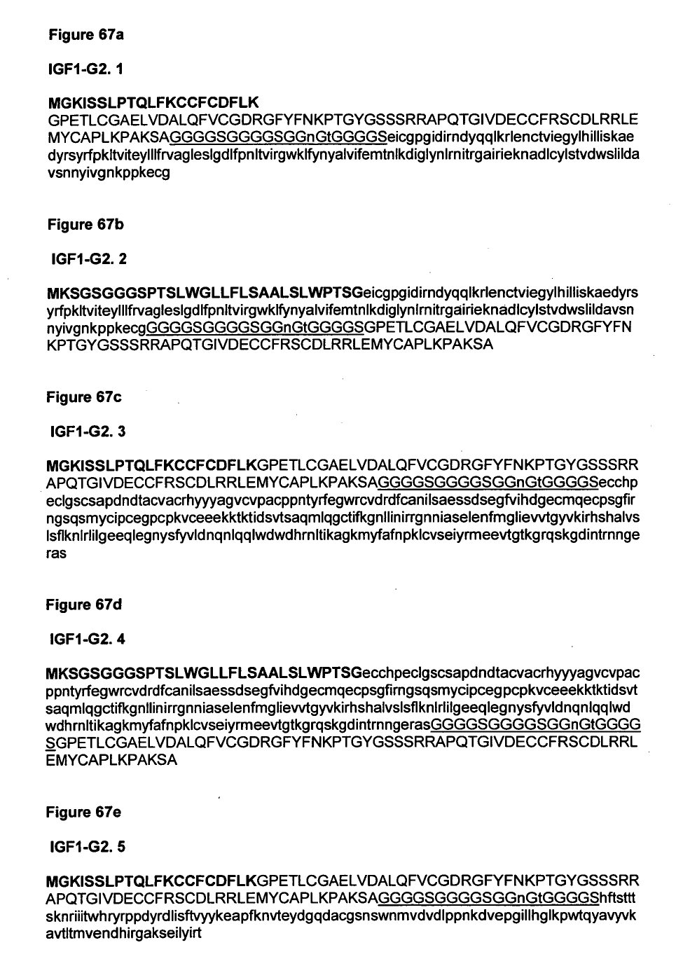

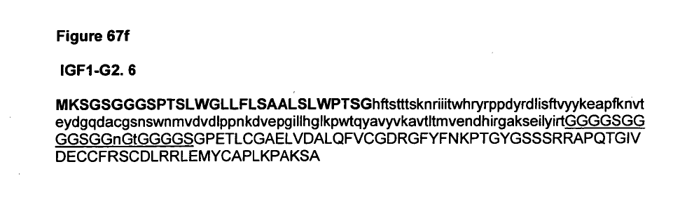

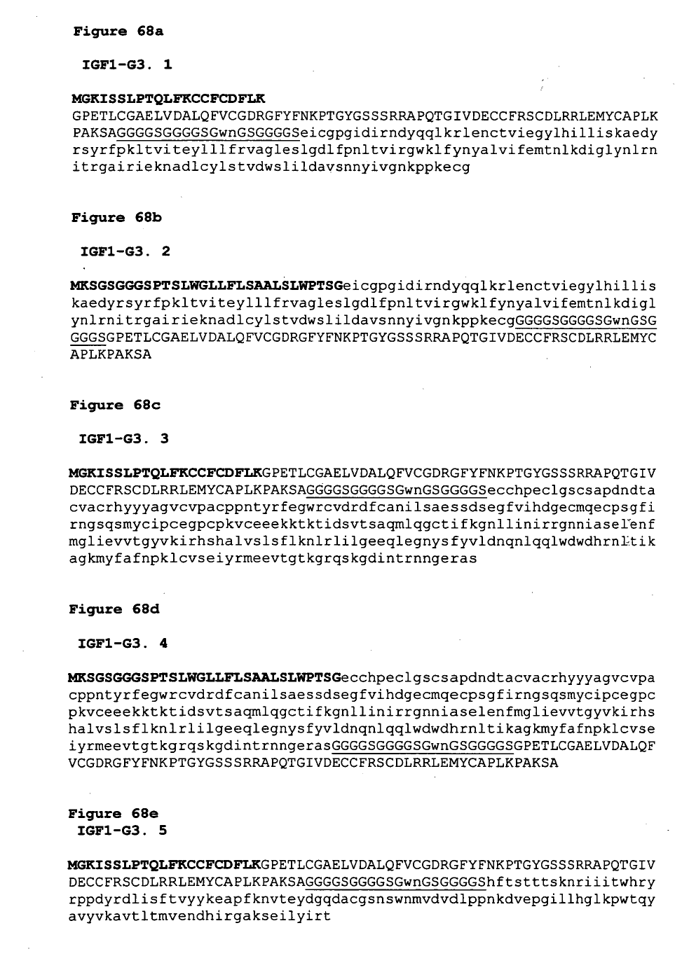

In a preferred embodiment of the invention said polypeptide comprises or consists of an amino acid sequence selected from the group consisting of the sequences represented in FIG. 67a, 67b, 67c, 67d, 67e or 67f.

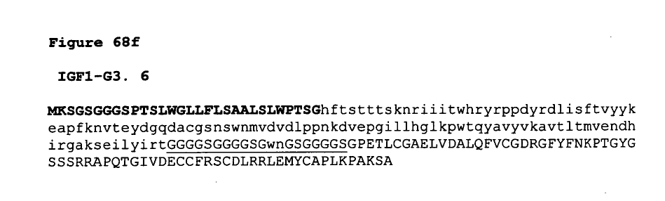

In a preferred embodiment of the invention said polypeptide comprises or consists of an amino acid sequence selected from the group consisting of the sequences represented in FIG. 68a, 68b, 68c, 68d, 68e or 68f.

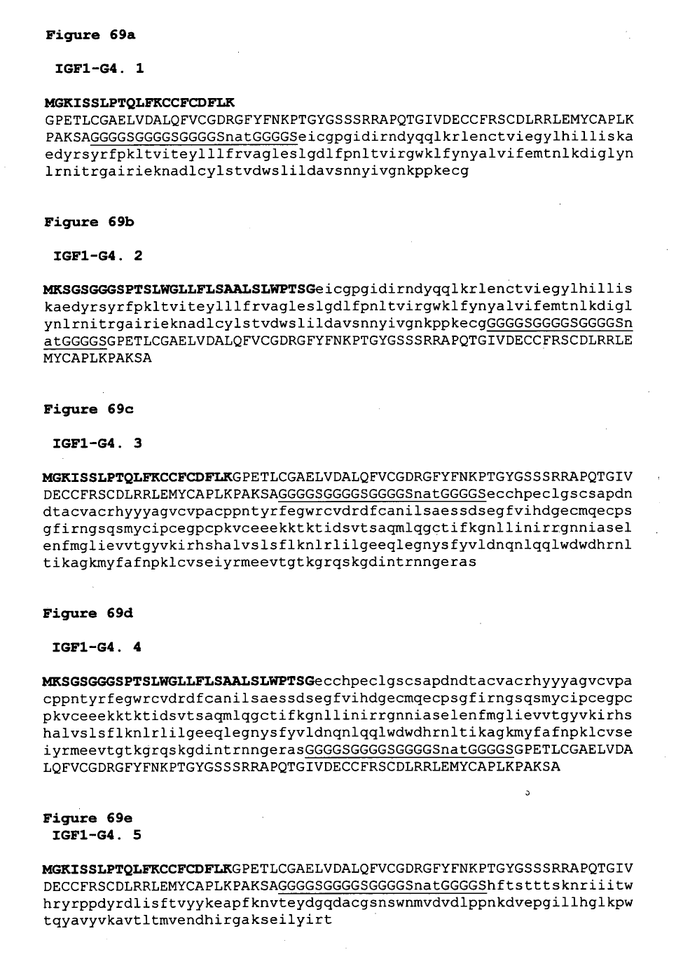

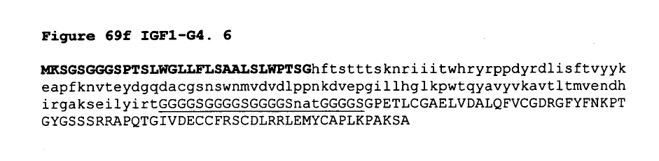

In a preferred embodiment of the invention said polypeptide comprises or consists of an amino acid sequence selected from the group consisting of the sequences represented in FIG. 69a, 69b, 69c, 69d, 69e or 69f.

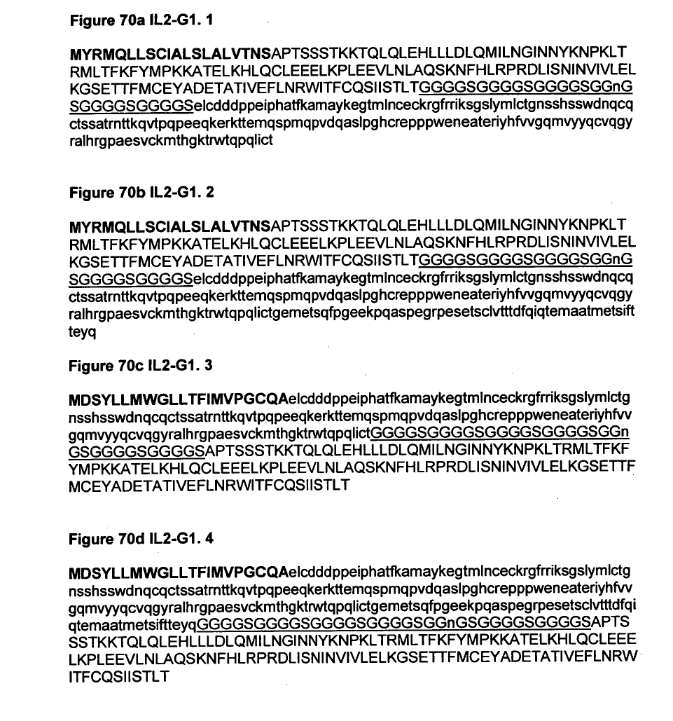

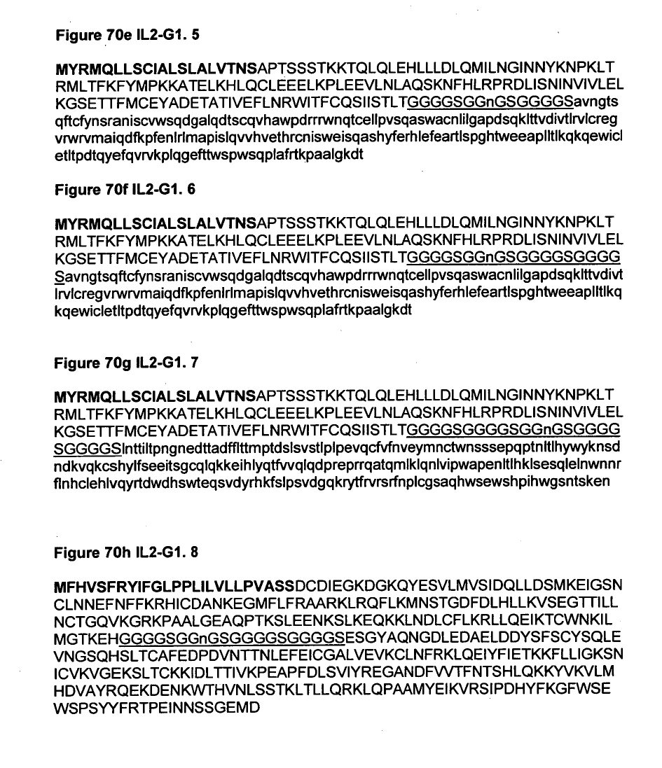

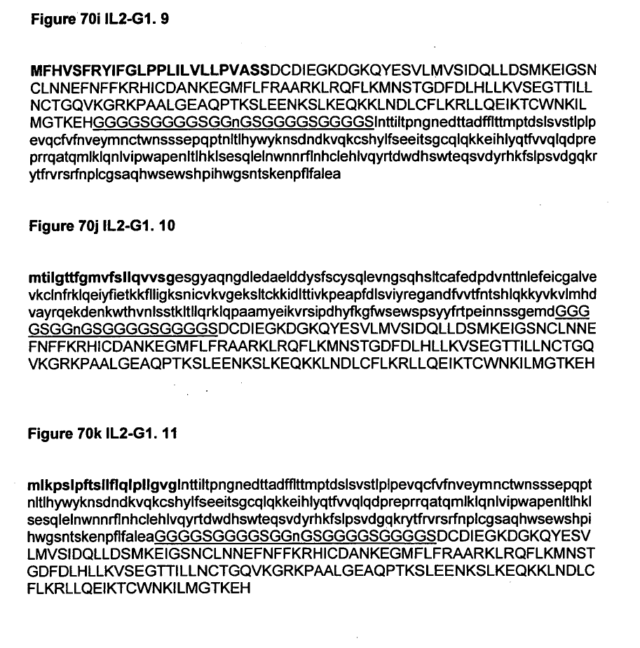

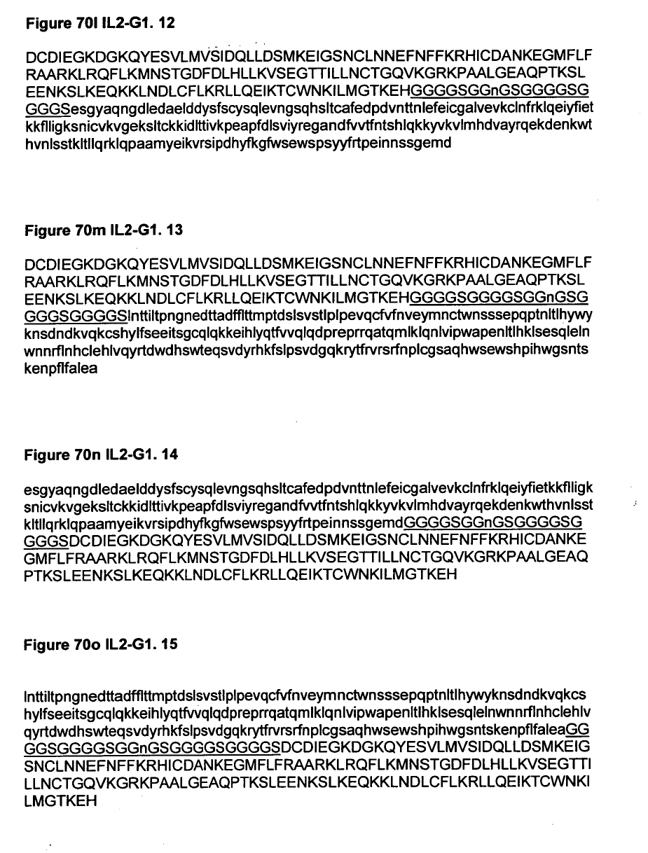

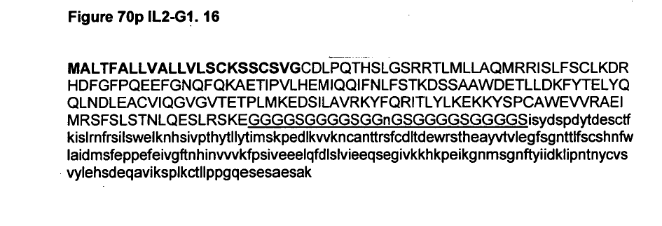

In a preferred embodiment of the invention said polypeptide comprises or consists of an amino acid sequence selected from the group consisting of the sequences represented in FIG. 70a, 70b, 70c, 70d, 70e, 70f, 70g, 70h, 70i, 70j, 70k, 70m, 70n, 70o and 70p.

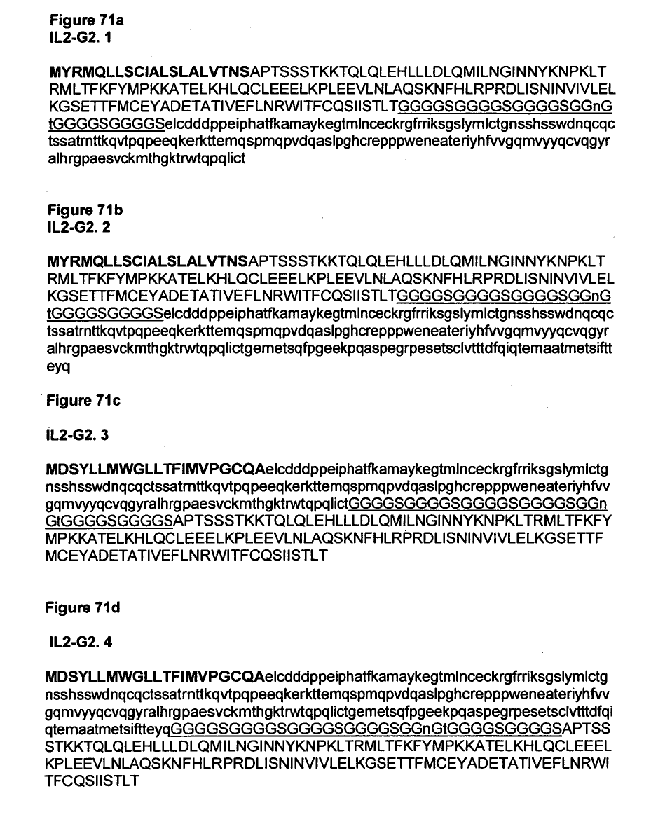

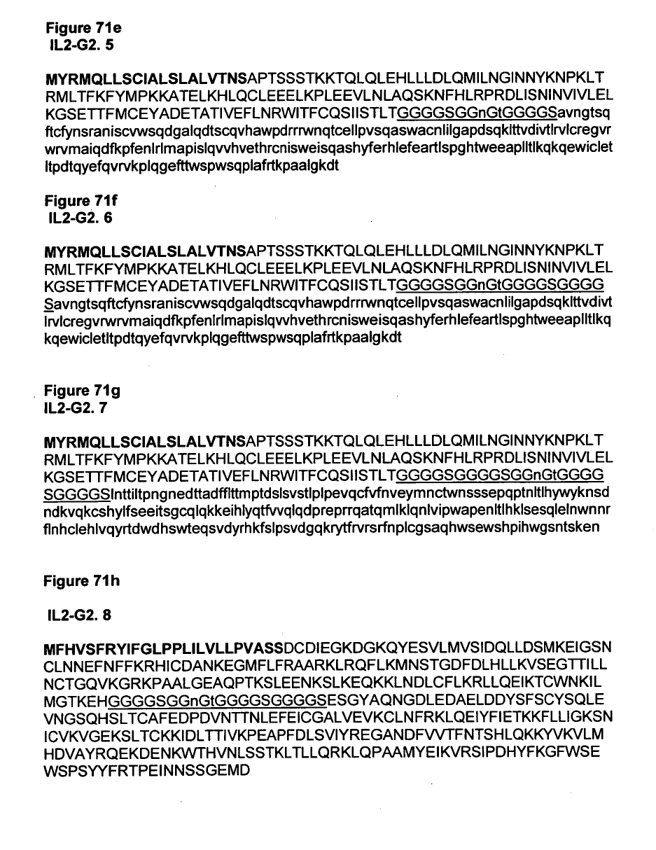

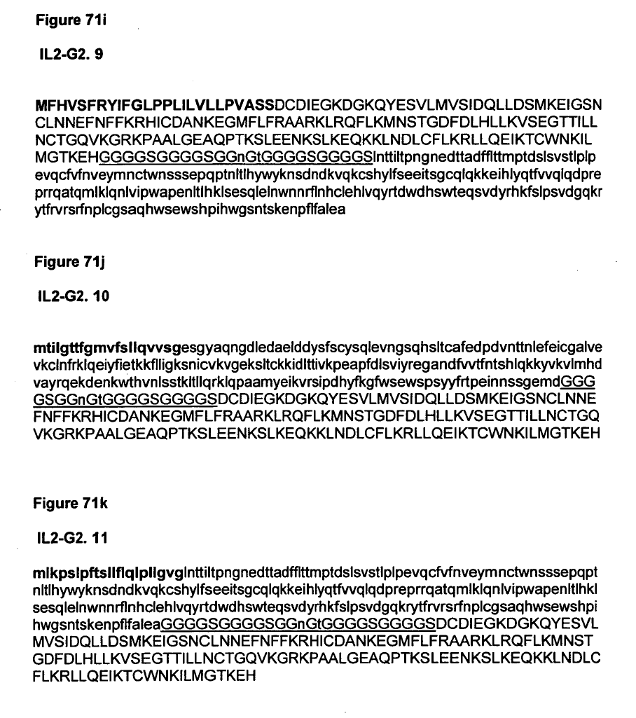

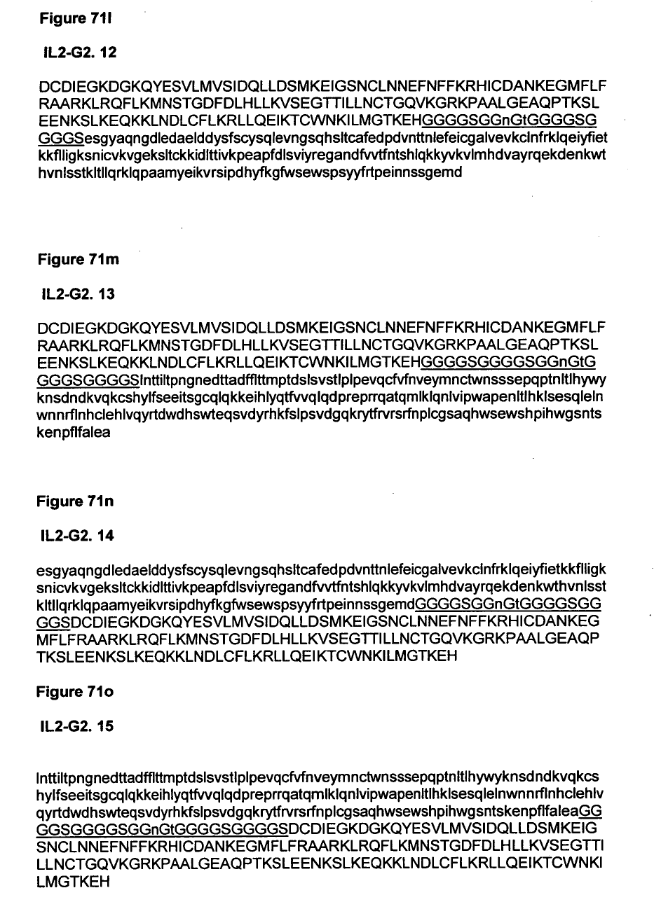

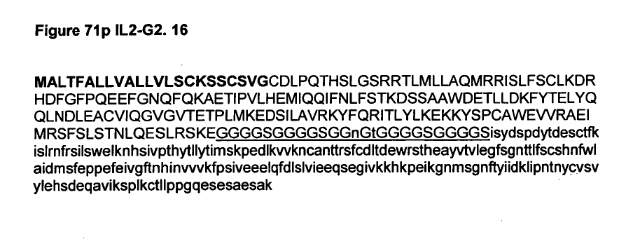

In a preferred embodiment of the invention said polypeptide comprises or consists of an amino acid sequence selected from the group consisting of the sequences represented in FIG. 71a, 71b, 71c, 71d, 71e, 71f, 71g, 71h, 71i, 71j, 71k, 71m, 71n, 71o and 71p.

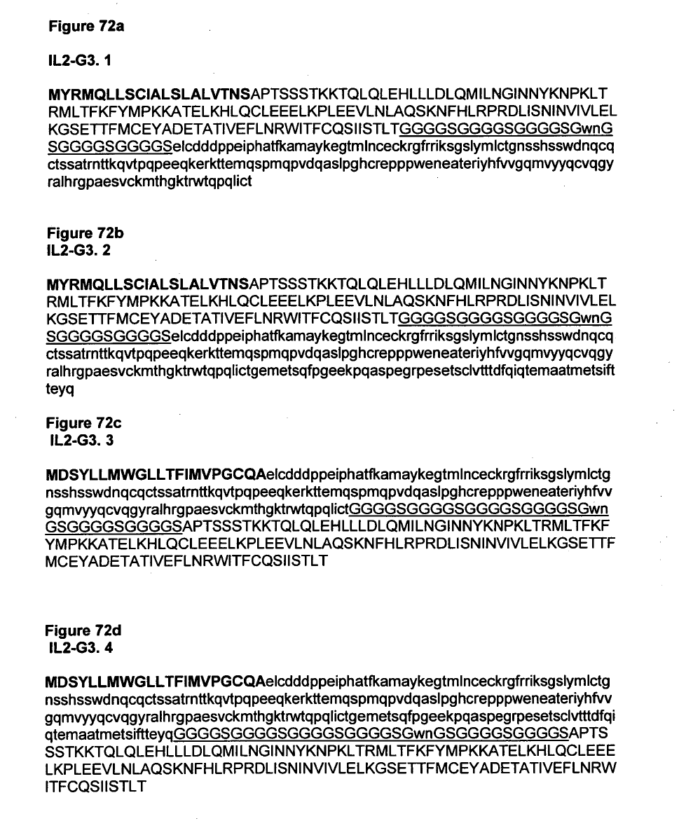

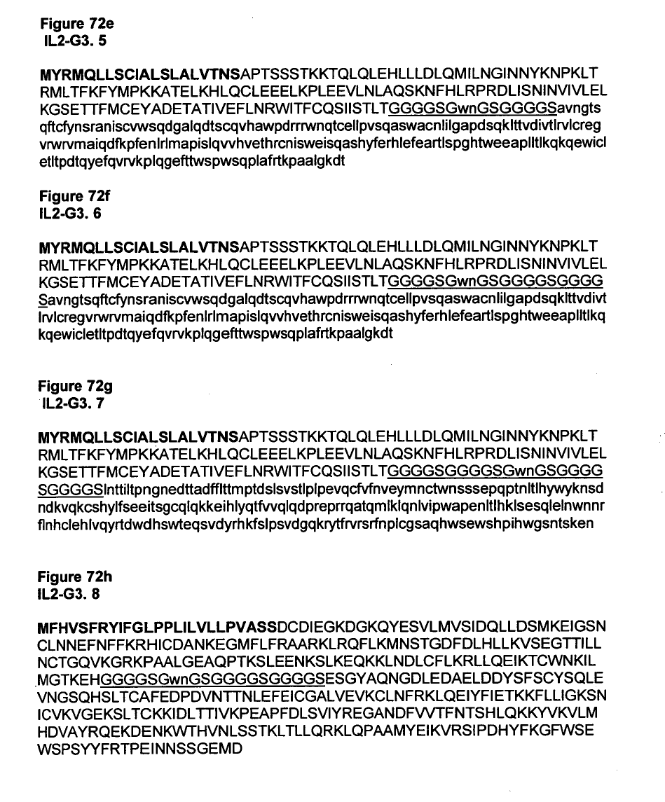

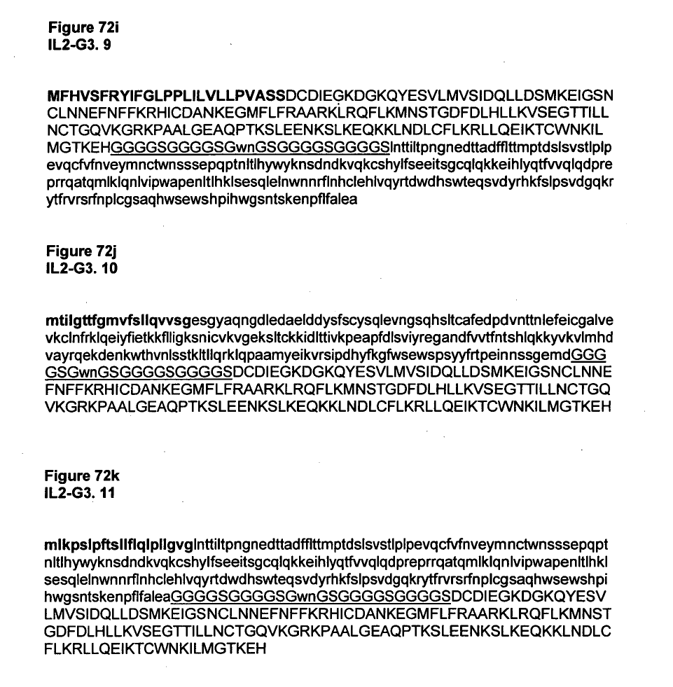

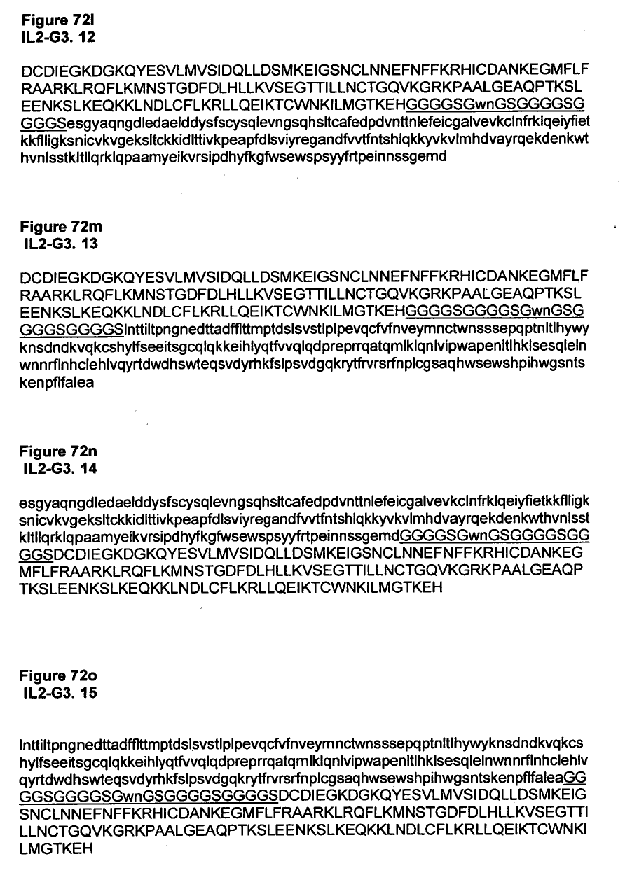

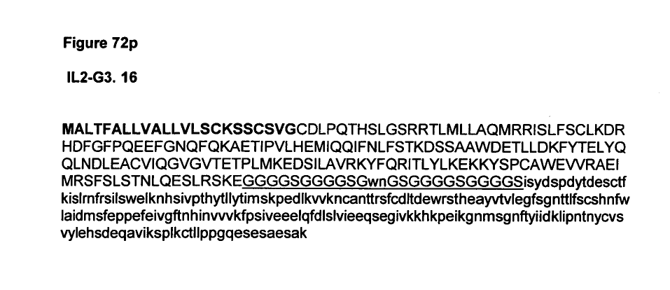

In a preferred embodiment of the invention said polypeptide comprises or consists of an amino acid sequence selected from the group consisting of the sequences represented in FIG. 72a, 72b, 72c, 72d, 72e, 72f, 72g, 72h, 72i, 72j, 72k, 72m, 72n, 72o and 72p.

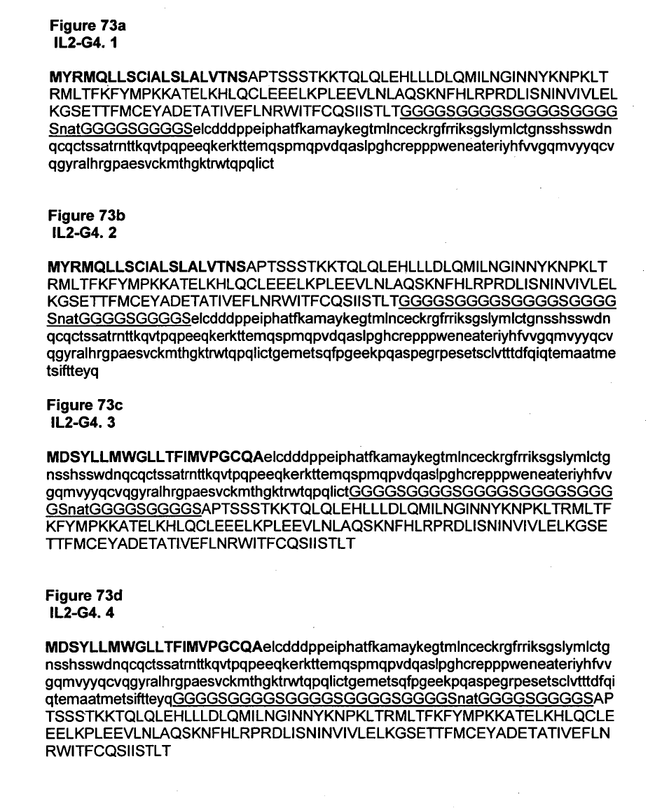

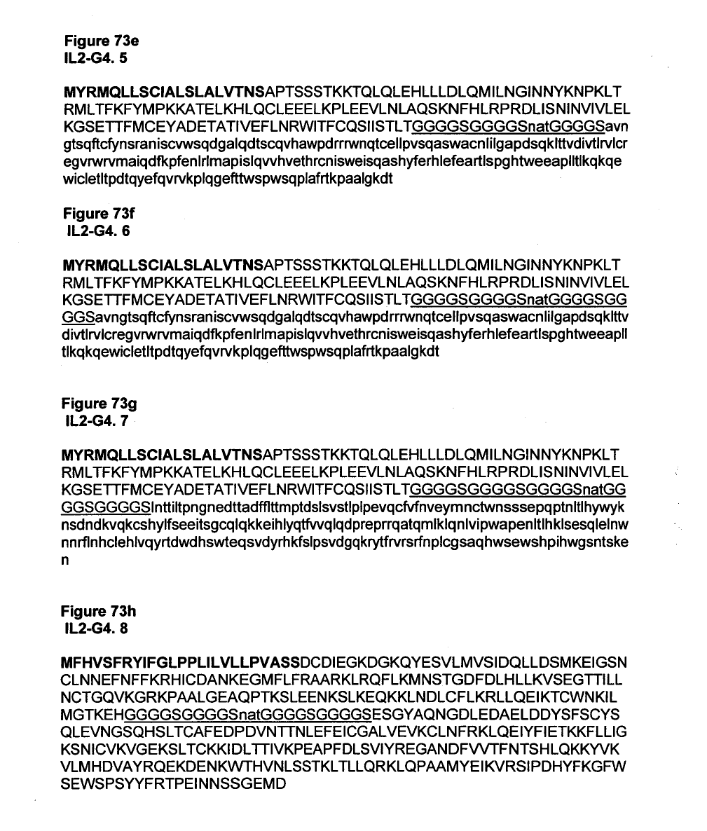

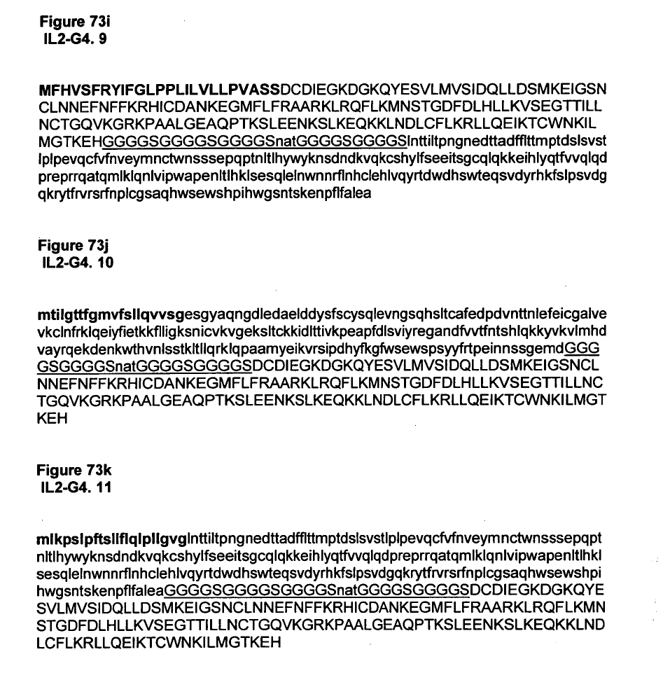

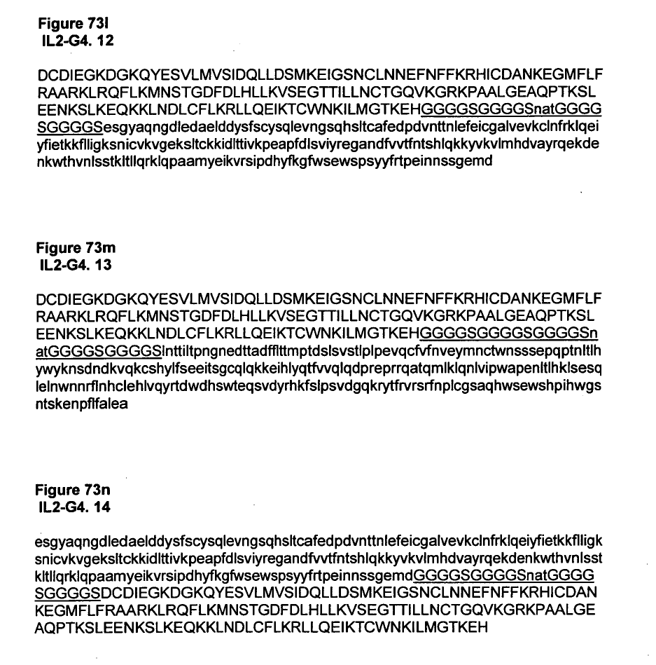

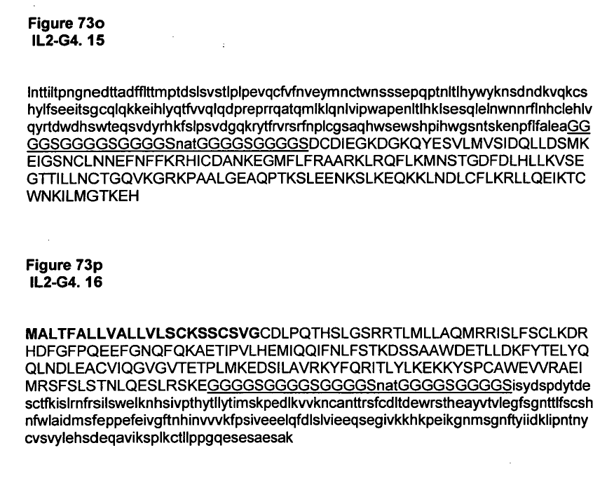

In a preferred embodiment of the invention said polypeptide comprises or consists of an amino acid sequence selected from the group consisting of the sequences represented in FIG. 73a, 73b, 73c, 73d, 73e, 73f, 73g, 73h, 73i, 73j, 73k, 73m, 73n, 73o and 73p.

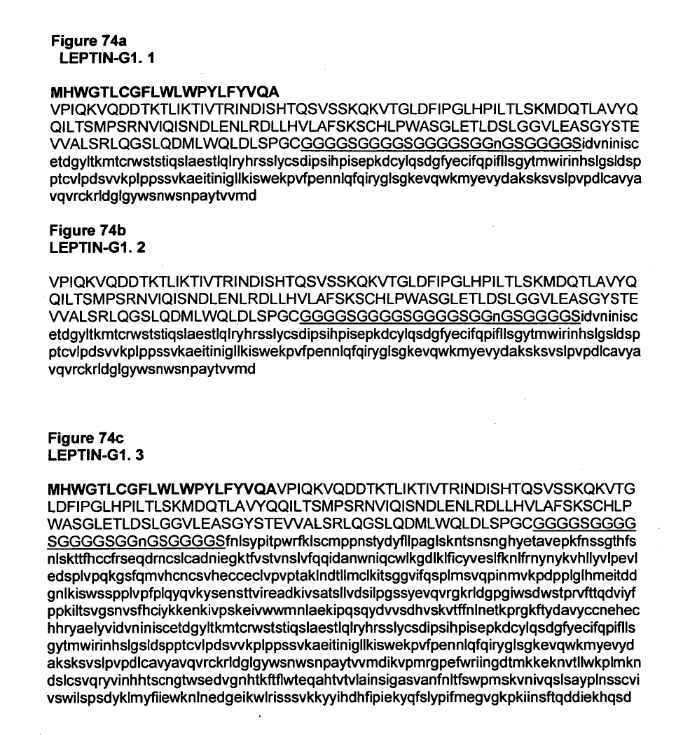

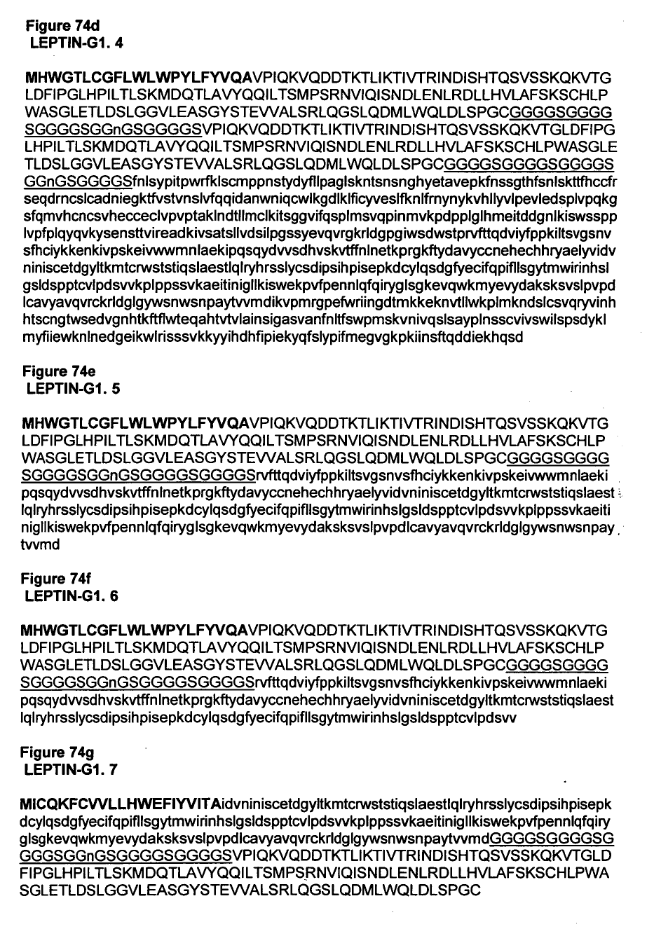

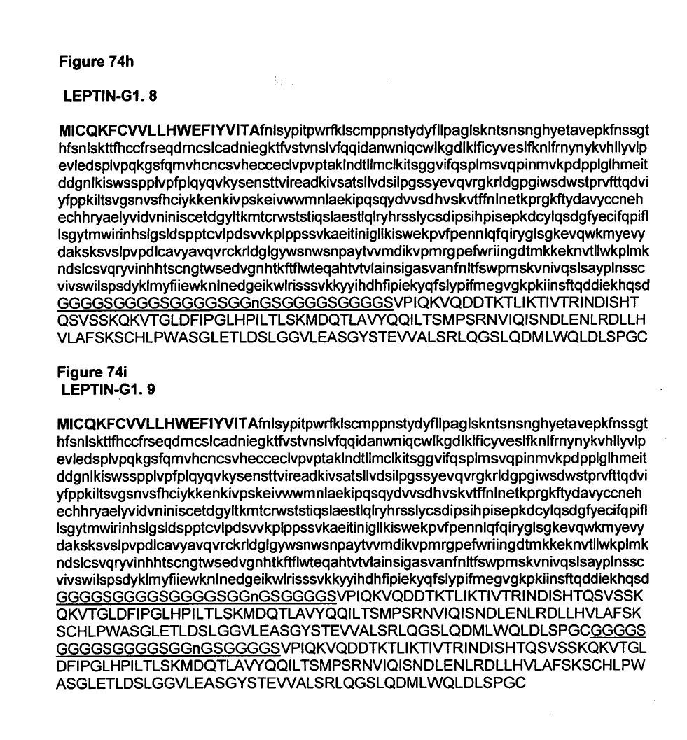

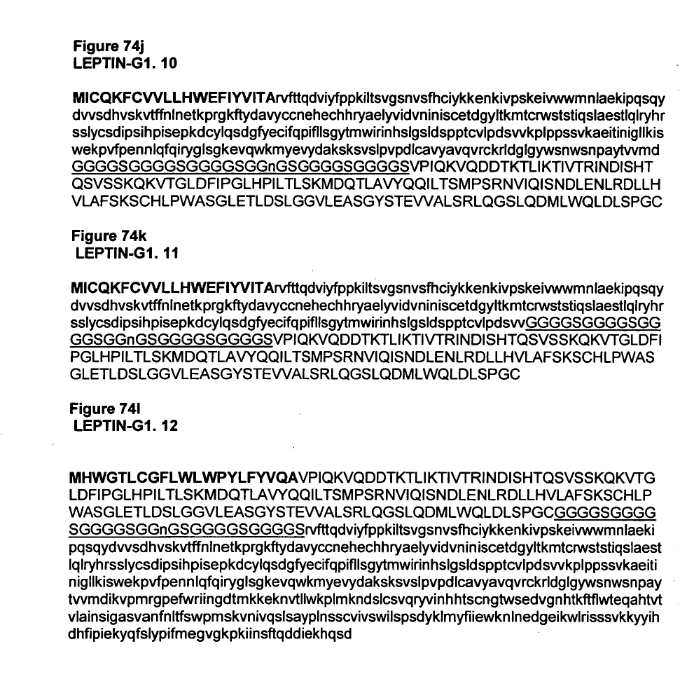

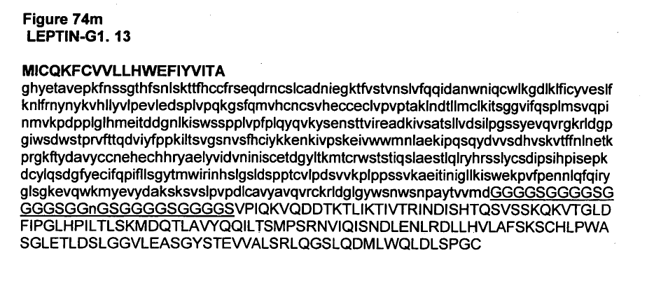

In a preferred embodiment of the invention said polypeptide comprises or consists of an amino acid sequence selected from the group consisting of the sequences represented in FIG. 74a, 74b, 74c, 74d, 74e, 74f, 74g, 74h, 74i, 74j, 74k or 74m.

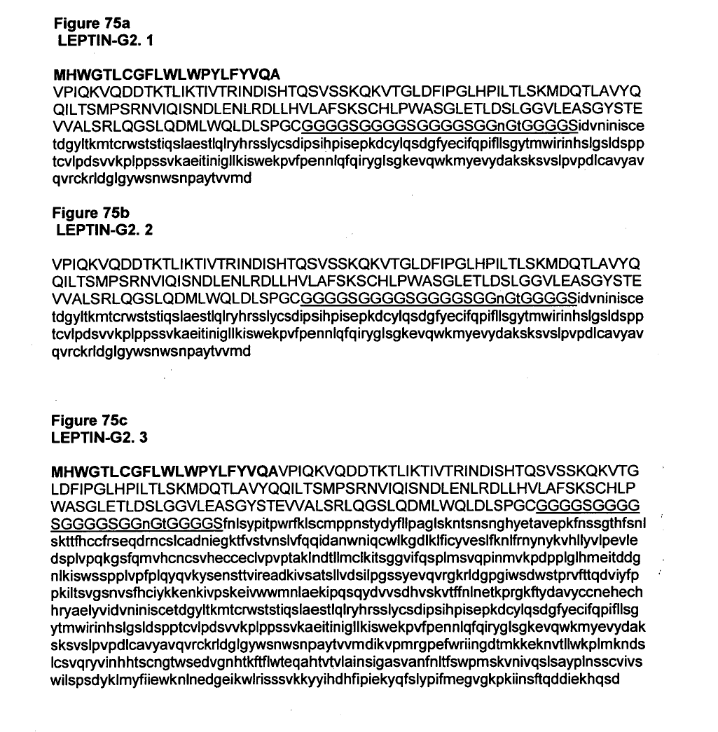

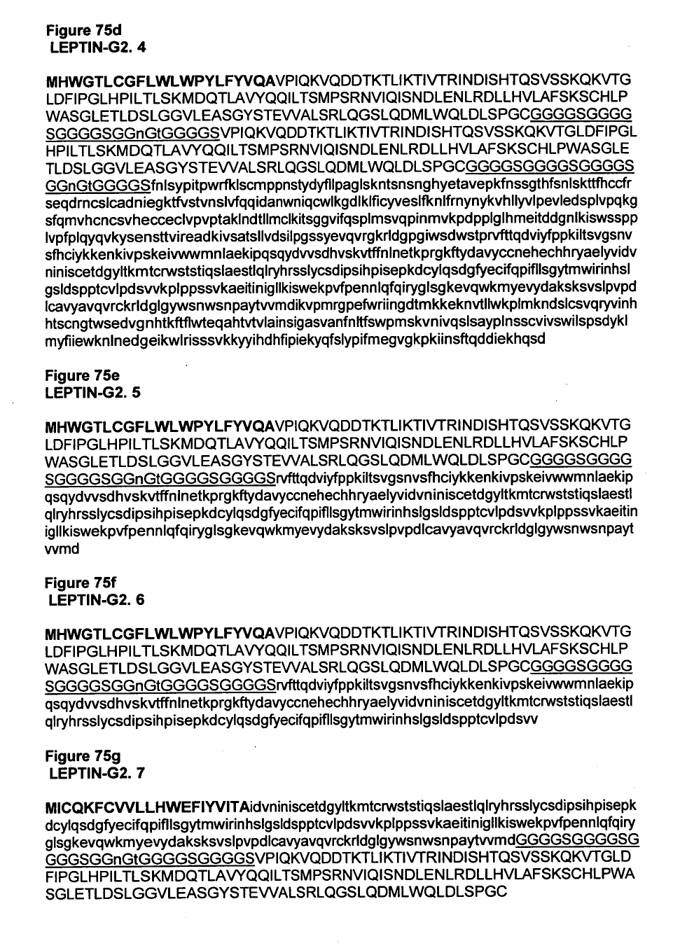

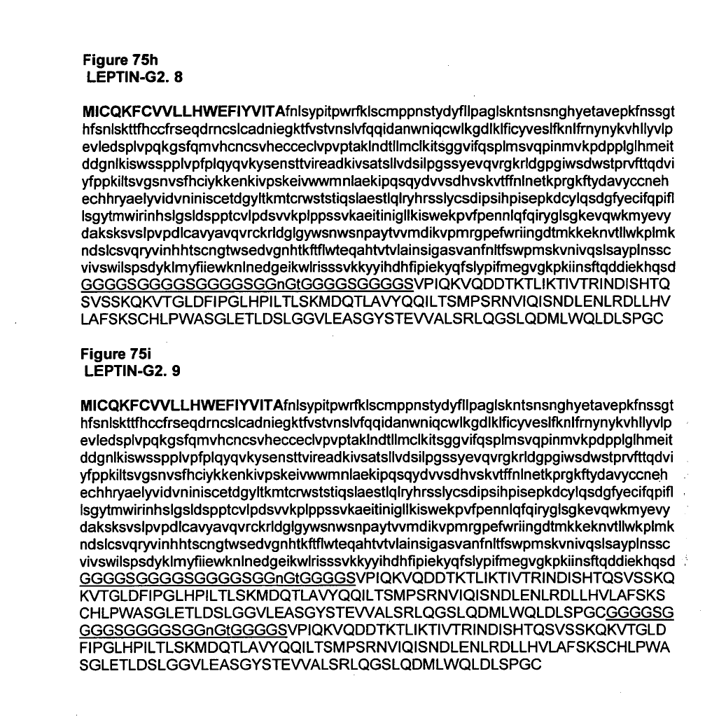

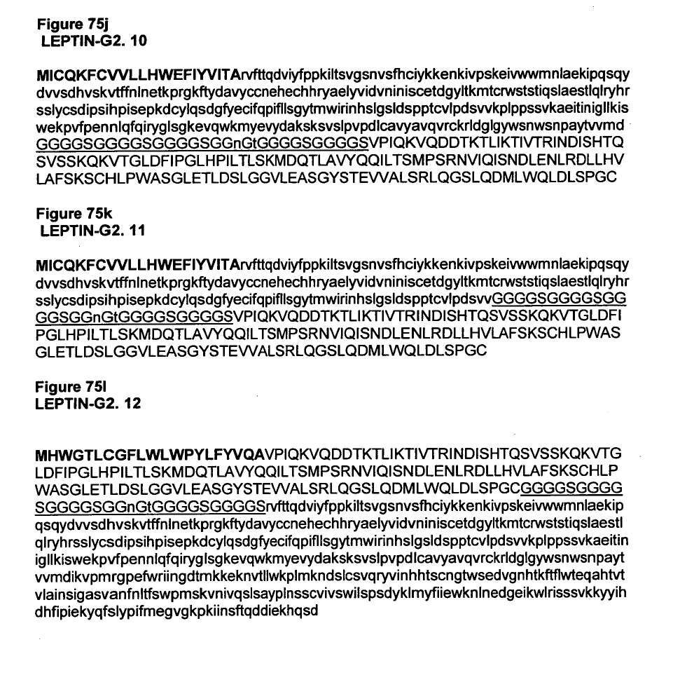

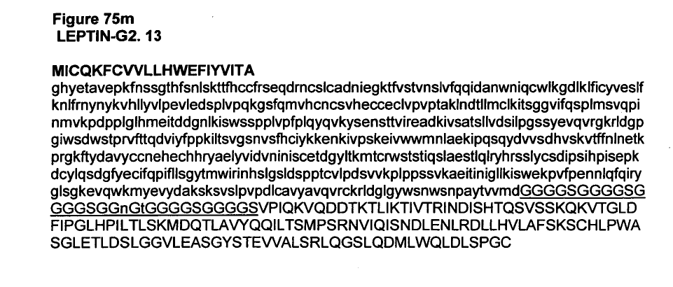

In a preferred embodiment of the invention said polypeptide comprises or consists of an amino acid sequence selected from the group consisting of the sequences represented in FIG. 75a, 75b, 75c, 75d, 75e, 75f, 75g, 75h, 75i, 75j, 75k or 75m.

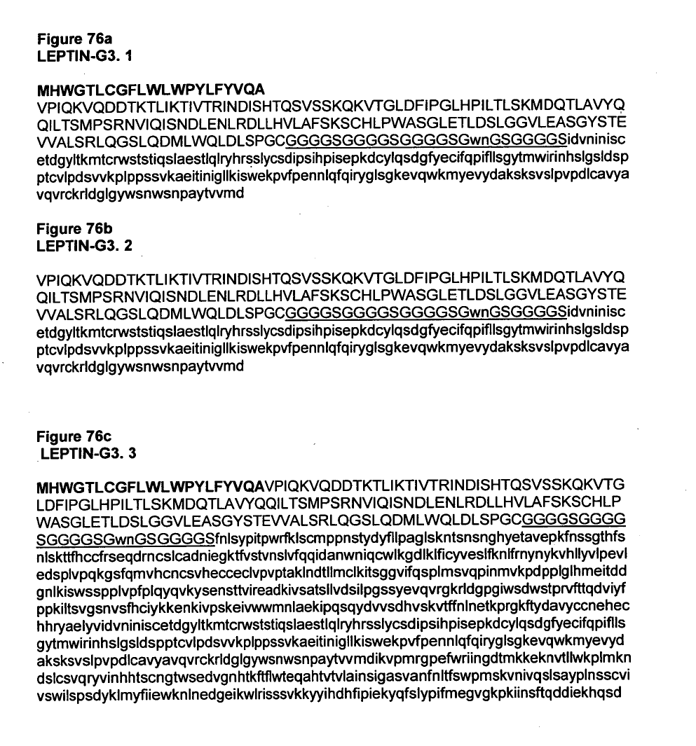

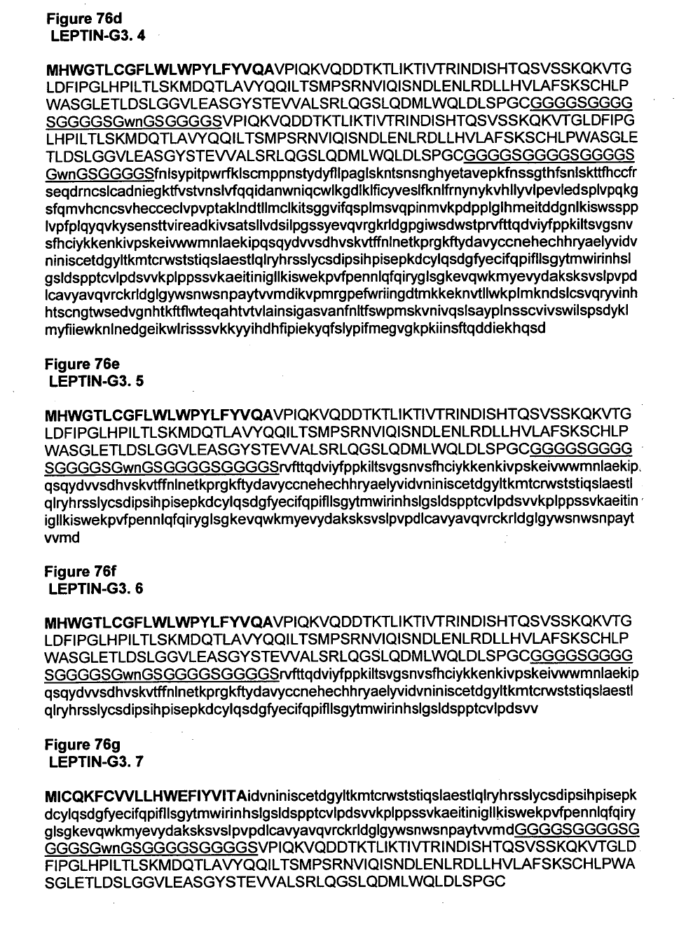

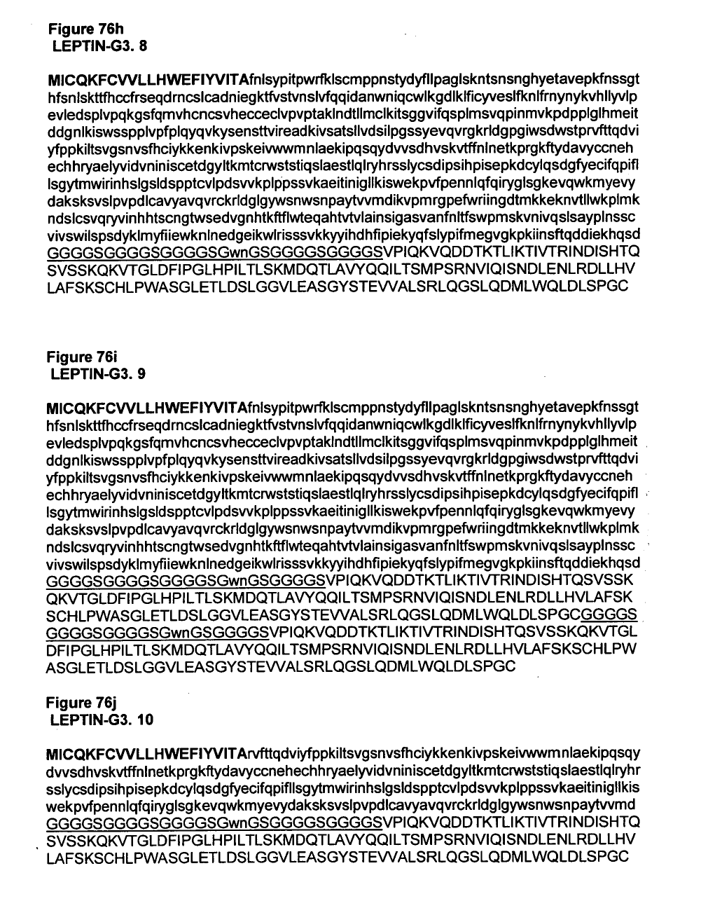

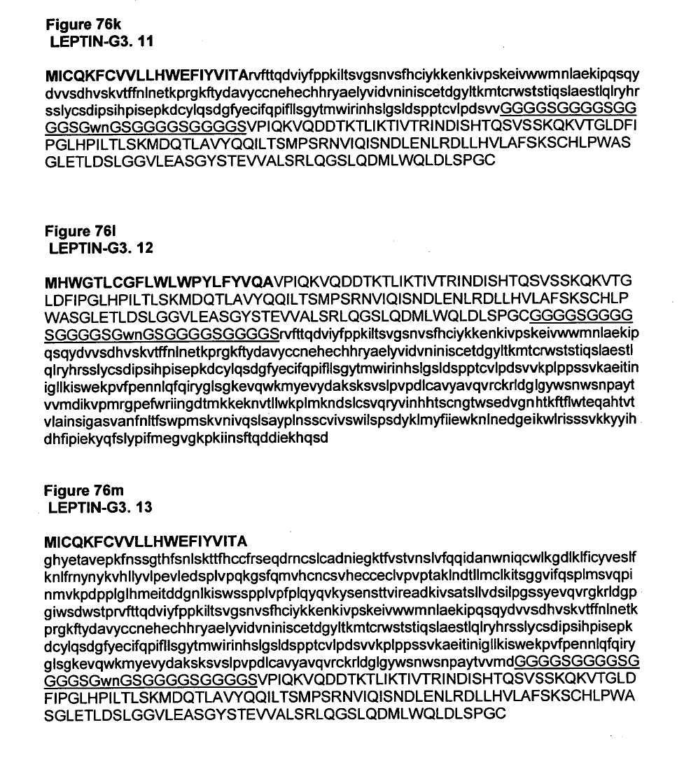

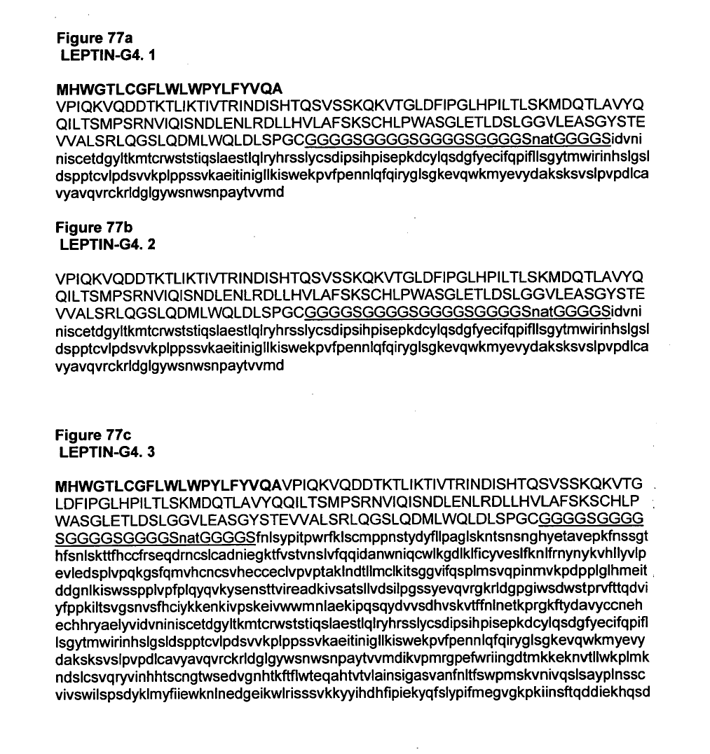

In a preferred embodiment of the invention said polypeptide comprises or consists of an amino acid sequence selected from the group consisting of the sequences represented in FIG. 76a, 76b, 76c, 76d, 76e, 76f, 76g, 76h, 76i, 76j, 76k or 76m.

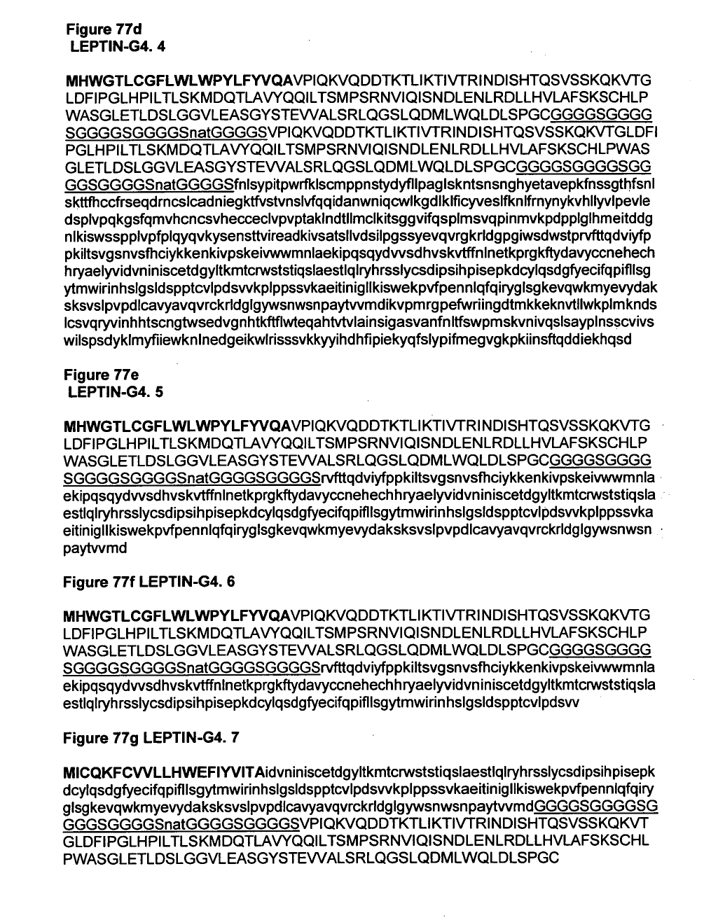

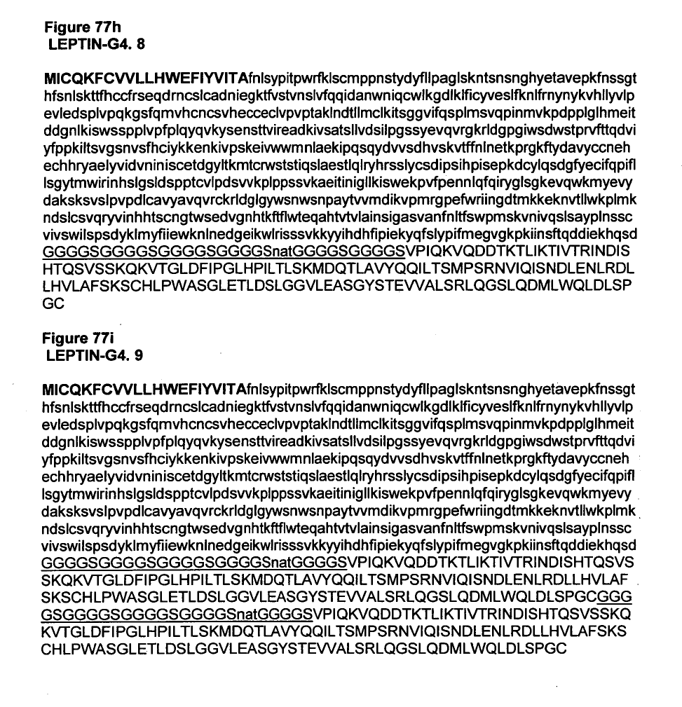

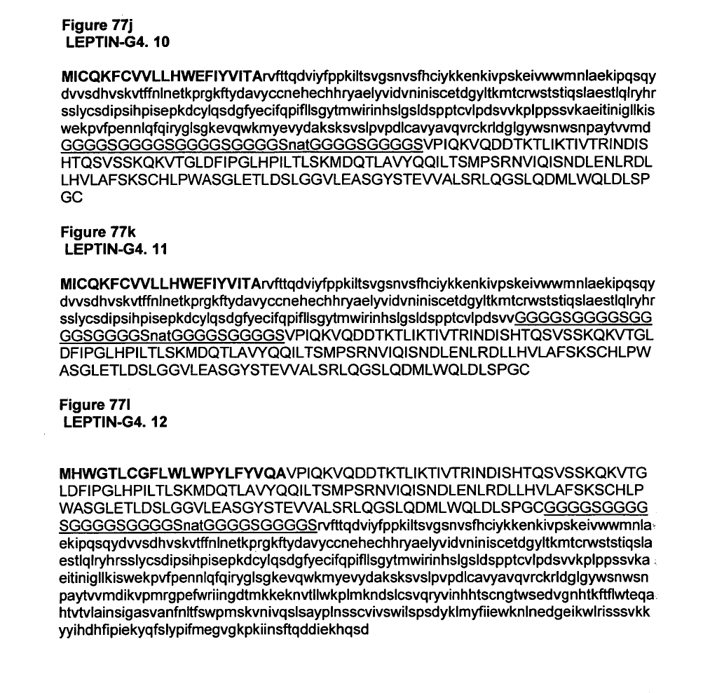

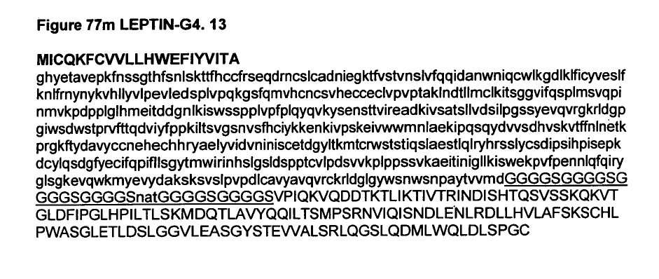

In a preferred embodiment of the invention said polypeptide comprises or consists of an amino acid sequence selected from the group consisting of the sequences represented in FIG. 77a, 77b, 77c, 77d, 77e, 77f, 77g, 77h, 77i, 77j, 77k or 77m.

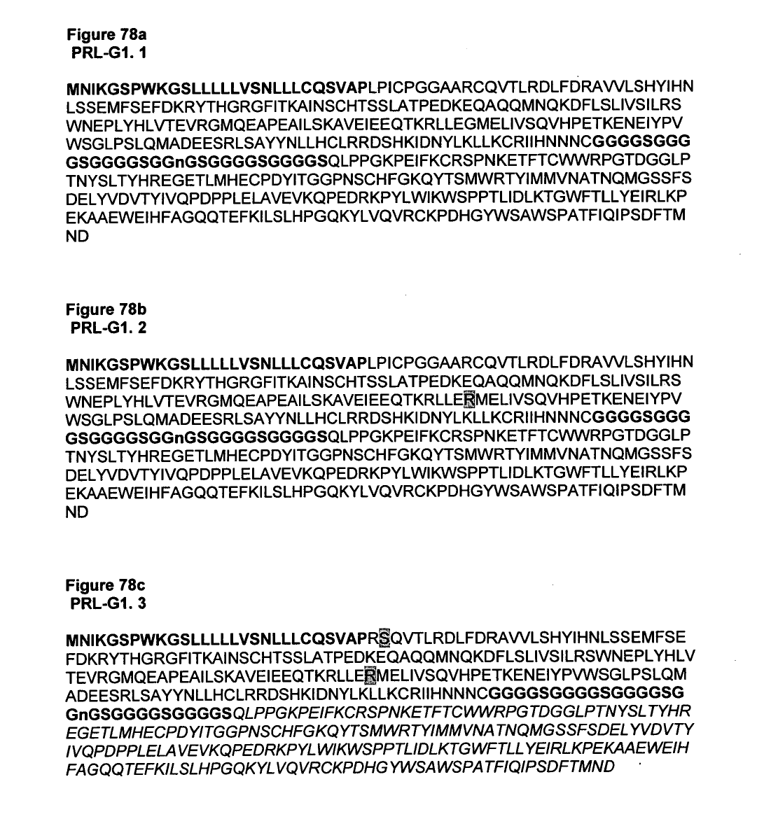

In a preferred embodiment of the invention said polypeptide comprises or consists of an amino acid sequence selected from the group consisting of the sequences represented in FIG. 78a, 78b or 78c.

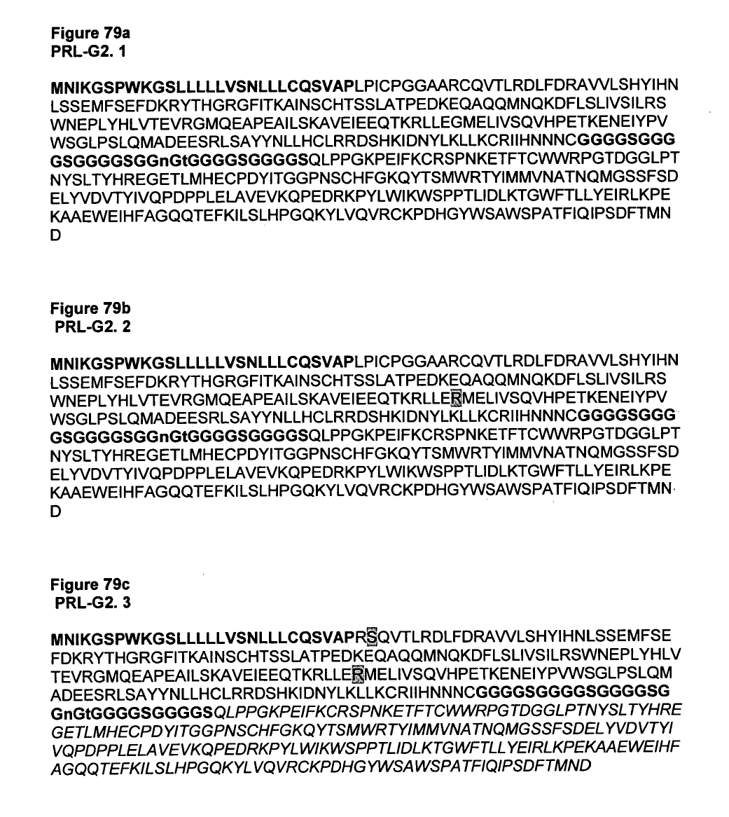

In a preferred embodiment of the invention said polypeptide comprises or consists of an amino acid sequence selected from the group consisting of the sequences represented in FIG. 79a, 79b or 79c.

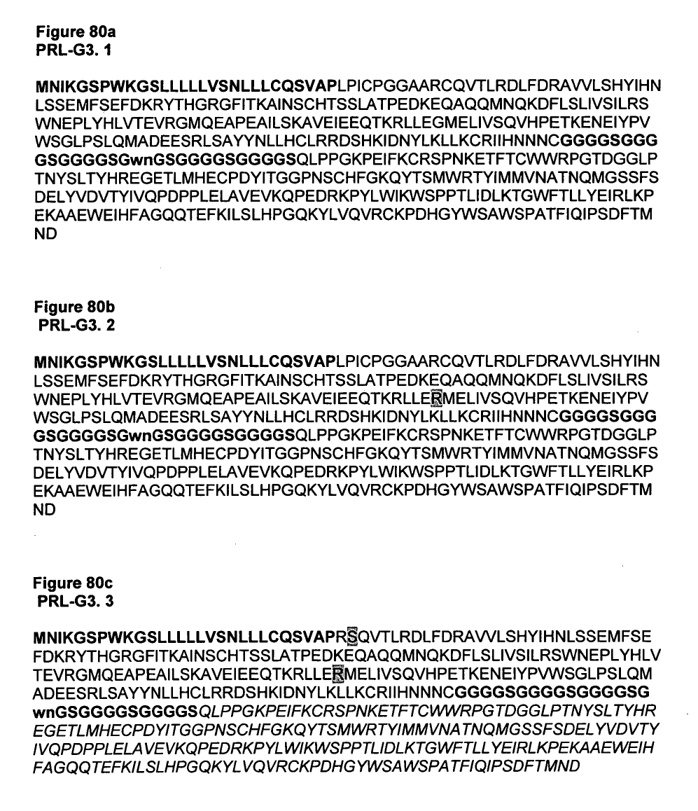

In a preferred embodiment of the invention said polypeptide comprises or consists of an amino acid sequence selected from the group consisting of the sequences represented in FIG. 80a, 80b or 80c.

In a preferred embodiment of the invention said peptide linker molecule is a flexible peptide linker.

In a preferred embodiment of the invention said peptide linker molecule comprises or consists of one copy of the glycosylation motif Asn-Xaa-Ser or Asn-Xaa-Thr where X is any amino acid except proline.

In a preferred embodiment of the invention said peptide linker molecule comprises at least 5 amino acid residues.

In a preferred embodiment of the invention said peptide linker comprises 5-50 amino acid residues.

In a further preferred embodiment of the invention said peptide linker consists of 5, 10, 15, 20, 25, 30, 35, 40, 45 or 50 amino acid residues.

In a preferred embodiment of the invention said peptide linker molecule comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr.

In a preferred embodiment of the invention said peptide linker comprises at least one copy of an amino acid motif selected from the group consisting of: Asn₁-Xaa₂-Ser₃ Xaa₄ Xaa₅ wherein Xaa₂ is any amino acid except proline; Xaa₁ Asn₂-Xaa₃-Ser₄ Xaa₅ wherein Xaa₃ is any amino acid except proline; Xaa₁ Xaa₂ Asn₃-Xaa₄-Ser₅ wherein Xaa₄ is any amino acid except proline; Asn₁-Xaa₂-Thr₃ Xaa₄ Xaa₅ wherein Xaa₂ is any amino acid except proline;

Xaa₁ Asn₂-Xaa₃-Thr₄ Xaa₅ wherein Xaa₃ is any amino acid except proline; and

Xaa₁ Xaa₂ Asn₃-Xaa₄-Thr₅ wherein Xaa₄ is any amino acid except proline.

Preferably said peptide linker comprises at least one copy of a motif selected from the group consisting of:

Asn₁-Xaa₂-Ser₃ Gly₄ Ser₅ wherein Xaa₂ is any amino acid except proline;

Asn₂-Xaa₃-Ser₄ Ser₅ wherein Xaa₃ is any amino acid except proline;

Gly₂ Asn₃-Xaa₄-Ser₅ wherein Xaa₄ is any amino acid except proline;

Asn₁-Xaa₂-Thr₃ Gly₄ Ser₅ wherein Xaa₂ is any amino acid except proline;

Gly, Asn₂-Xaa₃-Thr₄ Ser₅ wherein Xaa₃ is any amino acid except proline; and

Gly₂ Asn₃-Xaa₄-Thr₅ wherein Xaa₄ is any amino acid except proline.

In an alternative preferred embodiment of the invention said peptide linker comprises at least one copy of a motif selected from the group consisting of:

Asn₁-Xaa₂-Ser₃ Ser₄ Gly₅ wherein Xaa₂ is any amino acid except proline;

Ser₁ Asn₂-Xaa₃-Ser₄ Gly₅ wherein Xaa₃ is any amino acid except proline;

Ser₁ Ser₂ Asn₃-Xaa₄-Ser₅ wherein Xaa₄ is any amino acid except proline;

Asn₁-Xaa₂-Thr₃ Ser₄ Gly₅wherein Xaa₂ is any amino acid except proline;

Ser₁ Asn₂-Xaa₃-Thr₄ Gly₅ wherein Xaa₃ is any amino acid except proline; and

Ser₁ Ser₂ Asn₃-Xaa₄-Thr₅ wherein Xaa₄ is any amino acid except proline.

In a preferred embodiment of the invention said peptide linker molecule comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr and at least one copy of the motif (Gly Gly Gly Gly Ser) wherein said peptide linker is 5-50 amino acids.

In a preferred embodiment of the invention said peptide linker comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr and a copy of the motif (Ser Ser Ser Ser Gly) wherein said peptide linker is 5-50 amino acids.

In a preferred embodiment of the invention said fusion polypeptide linker is modified by the addition of at least one sugar selected from the group consisting of: mannose, galactose, n-acetyl glucosamine, n-acetyl neuraminic, acid n-glycolyl neuraminic acid, n-acetyl galactosamine, fucose, glucose, rhamnose, xylose, or a combinations of sugars, for example in an oligosacharide or scaffolded system.

Suitable carbohydrate moieties include monosaccharides, oligosaccharides and polysaccharides, and include any carbohydrate moiety that is present in naturally occurring glycoproteins or in biological systems. For example, optionally protected glycosyl or glycoside derivatives, for example optionally-protected glucosyl, glucoside, galactosyl or galactoside derivatives. Glycosyl and glycoside groups include both a and β groups. Suitable carbohydrate moieties include glucose, galactose, fucose, GlcNAc, GalNAc, sialic acid, and mannose, and oligosaccharides or polysaccharides comprising at least one glucose, galactose, fucose, GlcNAc, GalNAc, sialic acid, and/or mannose residue.

Any functional groups in the carbohydrate moiety may optionally be protected using protecting groups known in the art (see for example Greene et al, “Protecting groups in organic synthesis”, 2nd Edition, Wiley, New York, 1991, the disclosure of which is hereby incorporated by reference). Suitable protecting groups for any —OH groups in the carbohydrate moiety include acetate (Ac), benzyl (Bn), silyl (for example tert-butyl dimethylsilyl (TBDMSi) and tert-butyldiphenylsilyl (TMDPSi)), acetals, ketals, and methoxymethyl (MOM). Any protecting groups may be removed before or after attachment of the carbohydrate moiety to the peptide linker.

In a preferred embodiment of the invention said sugars are unprotected.

Particularly preferred carbohydrate moieties include Glc(Ac)₄β-, Glc(Bn)₄β-, Gal(Ac)₄β-, Gal(Bn)₄β-, Glc(Ac)₄α(1,4)Glc(Ac)₃α(1,4)Glc(Ac)₄β-, β-Glc, β-Gal, -Et-β-Gal,-Et-β-Glc, Et-α-Glc, -Et-α-Man, -Et-Lac, -β-Glc(Ac)₂, -β-Glc(Ac)₃, -Et-α-Glc(Ac)₂, -Et-α-Glc(Ac)₃, -Et-α-Glc(Ac)₄, -Et-β-Glc(Ac)₂, -Et-β-Glc(Ac)₃, -Et-β-Glc(Ac)₄, -Et-α-Man(Ac)₃, -Et-α-Man(Ac)₄, -Et-β-Gal(Ac)₃, -Et-β-Gal(Ac)₄, -Et-Lac(Ac)₅, -Et-Lac(Ac)₆, -Et-Lac(Ac)₇, and their deprotected equivalents.

Preferably, any saccharide units making up the carbohydrate moiety which are derived from naturally occurring sugars will each be in the naturally occurring enantiomeric form, which may be either the D-form (e.g. D-glucose or D-galactose), or the L-form (e.g. L-rhamnose or L-fucose). Any anomeric linkages may be α- or β-linkages.

According to a further aspect of the invention there is provided a nucleic acid molecule that encodes a polypeptide comprising a peptide linker capable of being glycosylated.

According to a further aspect of the invention there is provided a vector comprising a nucleic acid molecule according to the invention.

In a preferred embodiment of the invention said vector is an expression vector adapted to express the nucleic acid molecule according to the invention.

A vector including nucleic acid (s) according to the invention need not include a promoter or other regulatory sequence, particularly if the vector is to be used to introduce the nucleic acid into cells for recombination into the genome for stable transfection.

Preferably the nucleic acid in the vector is operably linked to an appropriate promoter or other regulatory elements for transcription in a host cell. The vector may be a bi-functional expression vector which functions in multiple hosts. By “promoter” is meant a nucleotide sequence upstream from the transcriptional initiation site and which contains all the regulatory regions required for transcription. Suitable promoters include constitutive, tissue-specific, inducible, developmental or other promoters for expression in eukaryotic or prokaryotic cells. “Operably linked” means joined as part of the same nucleic acid molecule, suitably positioned and oriented for transcription to be initiated from the promoter. DNA operably linked to a promoter is “under transcriptional initiation regulation” of the promoter.

In a preferred embodiment the promoter is a constitutive, an inducible or regulatable promoter.

According to a further aspect of the invention there is provided a cell transfected or transformed with a nucleic acid molecule or vector according to the invention.

Preferably said cell is a eukaryotic cell. Alternatively said cell is a prokaryotic cell.

In a preferred embodiment of the invention said cell is selected from the group consisting of; a fungal cell (e.g. Pichia spp, Saccharomyces spp, Neurospora spp); insect cell (e.g. Spodoptera spp); a mammalian cell (e.g. COS cell, CHO cell); a plant cell.

According to a further aspect of the invention there is provided a pharmaceutical composition comprising a polypeptide according to the invention including an excipient or carrier.

In a preferred embodiment of the invention said pharmaceutical composition is combined with a further therapeutic agent.

When administered the pharmaceutical composition of the present invention is administered in pharmaceutically acceptable preparations. Such preparations may routinely contain pharmaceutically acceptable concentrations of salt, buffering agents, preservatives, compatible carriers, and optionally other therapeutic agents.

The pharmaceutical compositions of the invention can be administered by any conventional route, including injection. The administration and application may, for example, be oral, intravenous, intraperitoneal, intramuscular, intracavity, intra-articuar, subcutaneous, topical (eyes), dermal (e.g a cream lipid soluble insert into skin or mucus membrane), transdermal, or intranasal.

Pharmaceutical compositions of the invention are administered in effective amounts. An “effective amount” is that amount of pharmaceuticals/compositions that alone, or together with further doses or synergistic drugs, produces the desired response. This may involve only slowing the progression of the disease temporarily, although more preferably, it involves halting the progression of the disease permanently. This can be monitored by routine methods or can be monitored according to diagnostic methods.

The doses of the pharmaceuticals compositions administered to a subject can be chosen in accordance with different parameters, in particular in accordance with the mode of administration used and the state of the subject (i.e. age, sex). When administered, the pharmaceutical compositions of the invention are applied in pharmaceutically-acceptable amounts and in pharmaceutically-acceptable compositions. When used in medicine salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as syrup, elixir or an emulsion.

Compositions suitable for parenteral administration conveniently comprise a sterile aqueous or non-aqueous preparation that is preferably isotonic with the blood of the recipient. This preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1, 3-butane diol. Among the acceptable solvents that may be employed are water, Ringer's solution, and isotonic sodium chloride solution. In addition, sterile, fixed oils are conventionally employed as a solvent or suspending medium. For this purpose any bland fixed oil may be employed including synthetic mono-or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

An embodiment of the invention will now be described by example only and with reference to the following figures:

FIG. 1 is the amino acid sequence of human insulin-like growth factor 1;

FIG. 2 is the amino acid sequence of human IGF-1 receptor polypeptide extracellular domain;

FIG. 3 is the amino acid sequence of human IGF-2;

FIG. 4a is the amino acid sequence of IGF-2 receptor extracellular domain; FIG. 4b is the binding domain for IGF-2;

FIG. 5 is the amino acid sequence of human growth hormone;

FIG. 6 is the amino acid sequence of human growth hormone receptor extracellular domain;

FIG. 7 is the amino acid sequence of human leptin;

FIG. 8 is the amino acid sequence of human leptin receptor extracellular domain;

FIG. 9 is the amino acid sequence of human erythropoietin;

FIG. 10 is the amino acid sequence of human erythropoietin receptor extracellular domain;

FIG. 11 is the amino acid sequence of human prolactin;

FIG. 12 is the amino acid sequence of human prolactin receptor extracellular domain;

FIG. 13 is the amino acid sequence of human G-CSF;

FIG. 14 is the amino acid sequence of human G-CSF receptor extracellular domain;

FIG. 15 is the amino acid sequence of human somatostatin receptor 1;

FIG. 16 is the amino acid sequence of human FSH α subunit;

FIG. 17 is the amino acid sequence of human FSH β subunit;

FIG. 18 is the amino acid sequence of human FSH receptor;

FIG. 19 is the amino acid sequence of human LH β subunit;

FIG. 20 is the amino acid sequence of human LH receptor;

FIG. 21 is the amino acid sequence of human TSH β;

FIG. 22 is the amino acid sequence of human TSH receptor;

FIG. 23 illustrates growth hormone tandem GHT-G1: GH-GH tandem expressed in mammalian cells. N replaces G in central G4S. GH secretion signal shown in bold and lower case;

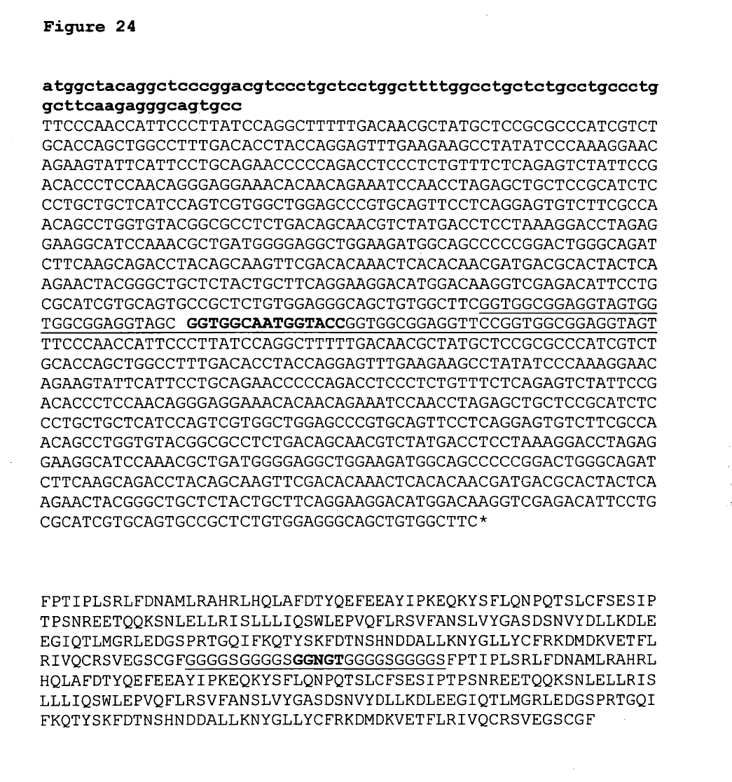

FIG. 24 illustrates growth hormone tandem GHT-G2: GH-GH tandem expressed in mammalian cells. N replaces G in central G4S, T replaces S. GH secretion signal shown in bold and lower case;

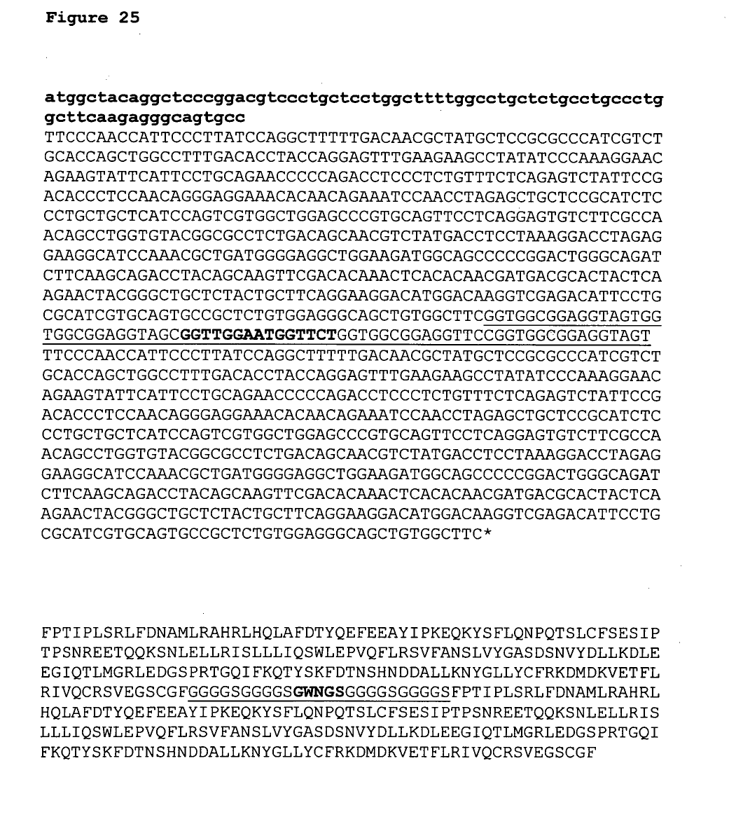

FIG. 25 illustrates growth hormone tandem GHT-G3: GH-GH tandem expressed in mammalian cells. N replaces G in central G4S; W replaces G before N.GH secretion signal shown in bold and lower case.

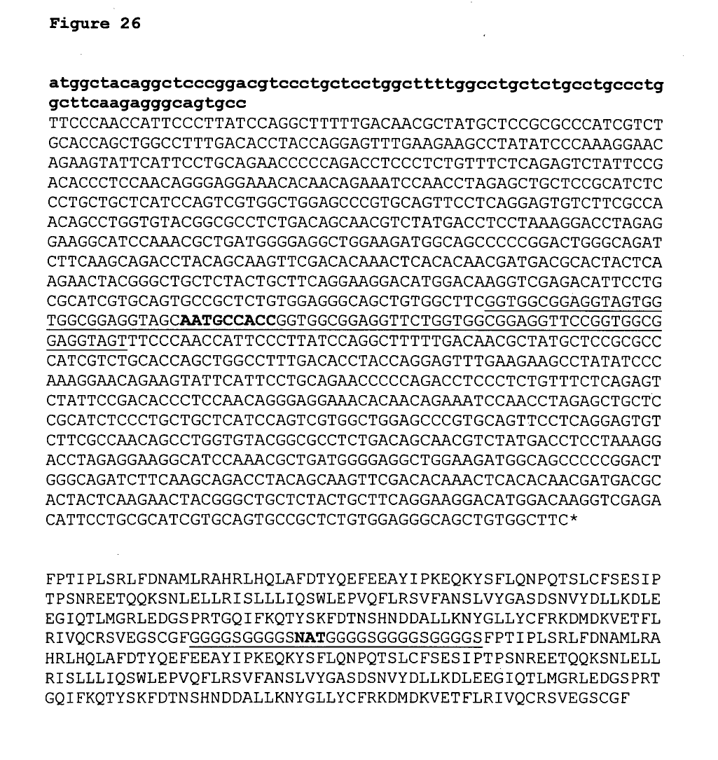

FIG. 26 illustrates growth hormone tandem GHT-G4: GH-GH tandem expressed in mammalian cells. A glycosylation signal (NAT) is placed between G4S units. GH secretion signal shown in bold and lower case.

FIG. 27 illustrates interferon β chimera 7B1-G1 N replaces G in central G4S;

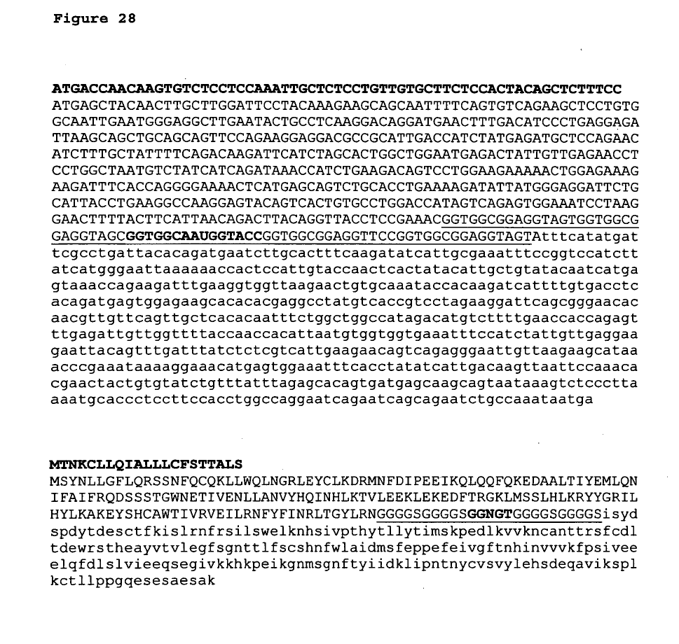

FIG. 28 illustrates interferon β chimera 7B1-G2 N replaces G in central G4S, T replaces S;

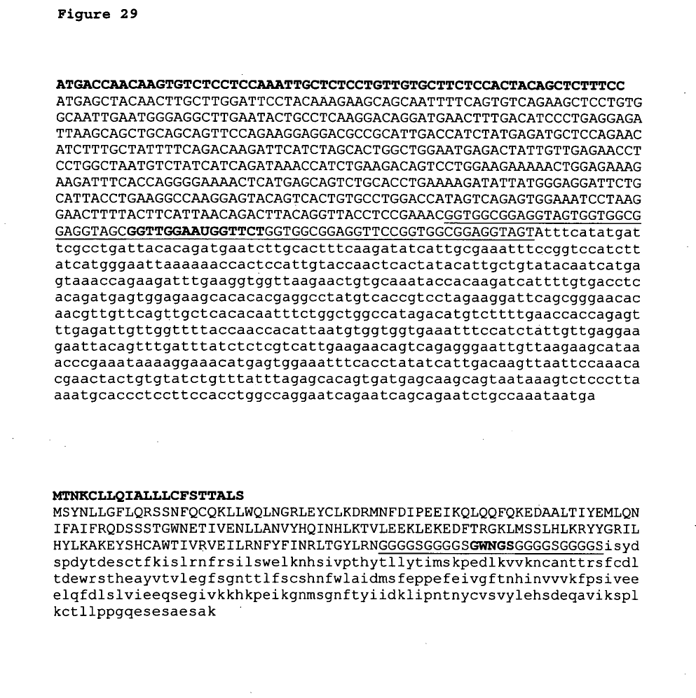

FIG. 29 illustrates interferon β chimera 7B1-G3 N replaces G in central G4S, W replaces G before N;

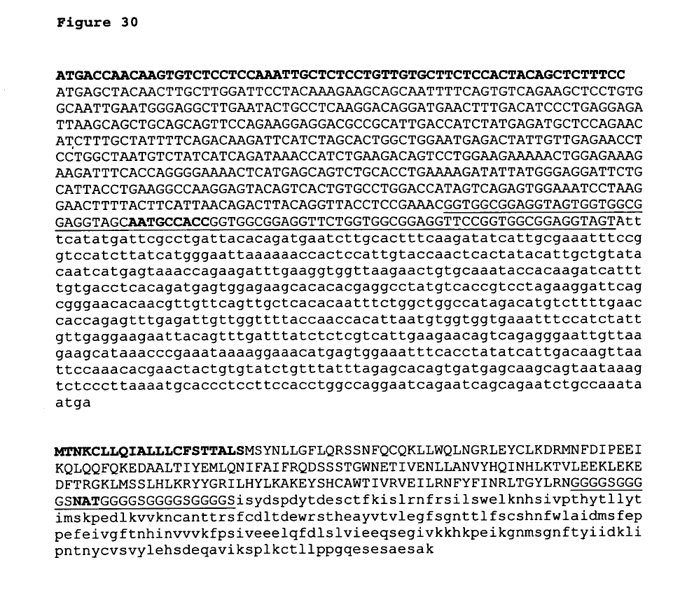

FIG. 30 illustrates interferon β chimera 7B1-G4 which incorporates a NAT glycosylation motif;

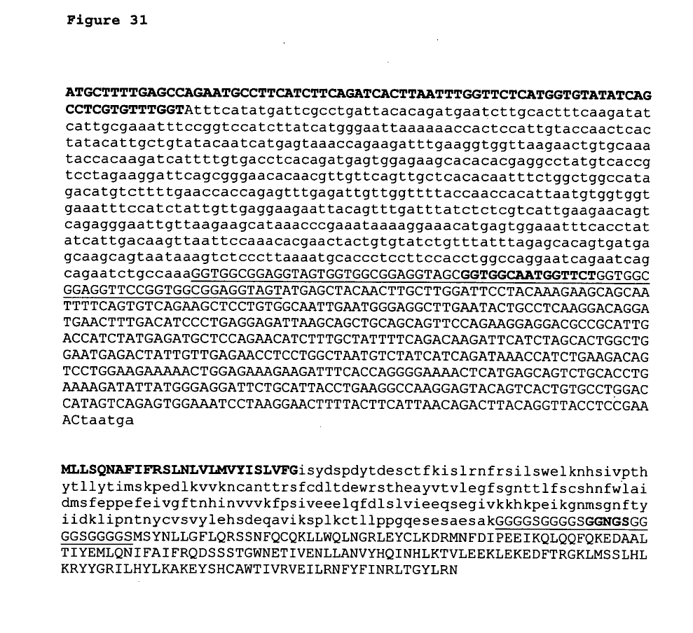

FIG. 31 illustrates interferon β chimera 7D1-G4N replaces G in central G4S;

FIG. 32 illustrates somatostatin-14 tandem [SMS14-G1]: N replaces G in central G4S.

FIG. 33 illustrates somatostatin-14 tandem [SMS14-G2]: N replaces G in central G4S, T replaces S

FIG. 34 illustrates somatostatin-14 tandem [SMS14-G3]: N replaces G in central G4S, W replaces G before N.

FIG. 35 illustrates somatostatin-14 tandem [SMS14-G4]: A glycosylation signal (NAT) is placed between G4S units.

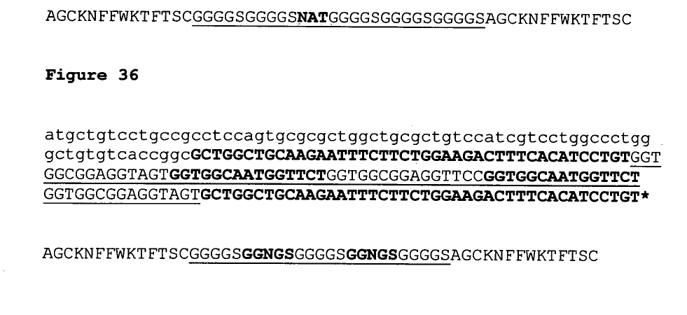

FIG. 36 illustrates somatostatin-14 tandem [SMS14-G6]: N replaces G in two G4S repeats;

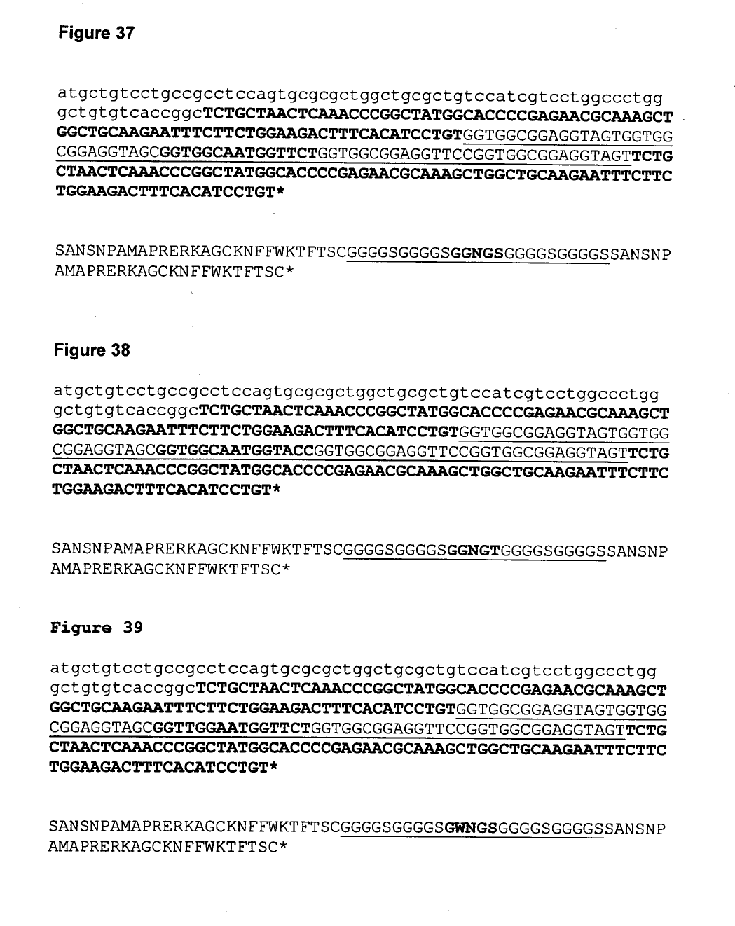

FIG. 37 illustrates somatostatin-28 tandem [SMS28-G1]: N replaces G in central G4S.

FIG. 38 illustrates somatostatin-28 tandem [SMS28-G2]: N replaces G in central G4S, T replaces S.

FIG. 39 illustrates somatostatin-28 tandem [SMS28-G3]:N replaces G in central G4S, W replaces G before N.

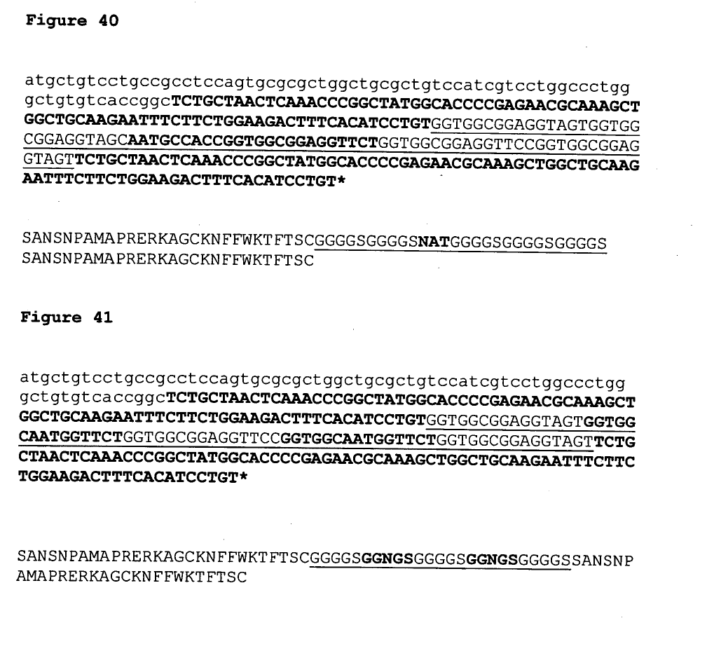

FIG. 40 illustrates somatostatin-28 tandem [SMS28-G4]: A glycosylation signal (NAT) is placed between G4S units

FIG. 41 illustrates somatostatin-28 tandem [SMS28-G6]:N replaces G in two G4S repeats

FIG. 42 illustrates somatostatin-28:somatostatin-14 tandem [SMS2814-G1]:N replaces G in central G4S.

FIG. 43 illustrates somatostatin-14:somatostatin-28 tandem [SMS1428-G1]:N replaces G in central G4S;

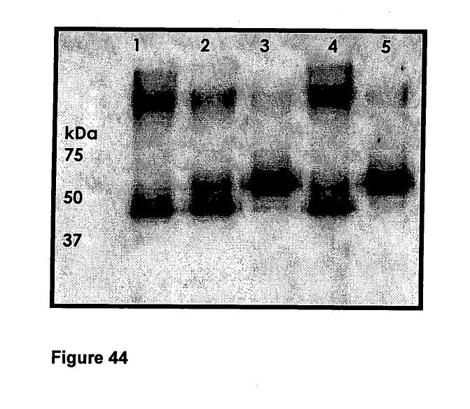

FIG. 44 shows western blot of CHO Flp-In expressed GH tandem molecules under non reducing conditions. Lane 1; GHT-0 (wild-type), Lane 2: GHT-1, Lane 3: GHT-2, Lane 4: GHT-3, Lane 5: GHT-4

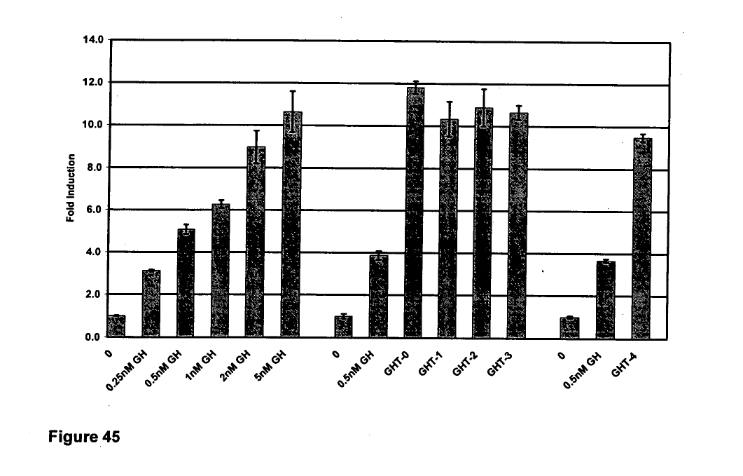

FIG. 45 Bioactivity of media samples taken from transfected CHO Fip-In cells expressing either wild-type GH tandem (GHT-0) or linker mutated GH tandem molecules (GHT-1-4). The bioassay is based on a STAT-5 reporter gene assay linked to luciferase expression, using Hek293 cells expressing full length GHR. All molecules studies exhibited GH activity in the assay;

FIG. 46a is the amino acid sequence of growth hormone fusion polypeptide 1B7-G1-V0 unprocessed; FIG. 46b is the amino acid sequence of growth hormone fusion polypeptide 1B7-G1-V0 processed; FIG. 46c is the amino acid sequence of growth hormone fusion polypeptide 1B7-G1-V1 unprocessed; FIG. 46d is the amino acid sequence of growth hormone fusion polypeptide 1B7-G1-V1 processed; FIG. 46e is the amino acid sequence of growth hormone fusion polypeptide 1B7-G1-V2 un processed; FIG. 46f is the amino acid sequence of growth hormone fusion polypeptide 1B7-G1-V2 processed;

FIG. 47a is the amino acid sequence of growth hormone fusion polypeptide 1B7-G2-V0 unprocessed; FIG. 47b is the amino acid sequence of growth hormone fusion polypeptide 1B7-G2-V0 processed; FIG. 47c is the amino acid sequence of growth hormone fusion polypeptide 1B7-G2-V1 unprocessed; FIG. 47d is the amino acid sequence of growth hormone fusion polypeptide 1B7-G2-V1 processed; FIG. 47e is the amino acid sequence of growth hormone fusion polypeptide 1B7-G2-V2 un processed; FIG. 47f is the amino acid sequence of growth hormone fusion polypeptide 1B7-G2-V2 processed;

FIG. 48a is the amino acid sequence of growth hormone fusion polypeptide 1B7-G3-V0 unprocessed; FIG. 48b is the amino acid sequence of growth hormone fusion polypeptide 1B7-G3-V0 processed; FIG. 48c is the amino acid sequence of growth hormone fusion polypeptide 1B7-G3-V1 unprocessed; FIG. 48d is the amino acid sequence of growth hormone fusion polypeptide 1B7-G3-V1 processed; FIG. 48e is the amino acid sequence of growth hormone fusion polypeptide 1B7-G3-V2 un processed; FIG. 48f is the amino acid sequence of growth hormone fusion polypeptide 1B7-G3-V2 processed;

FIG. 49a is the amino acid sequence of growth hormone fusion polypeptide 1B7-G4-V0 unprocessed; FIG. 49b is the amino acid sequence of growth hormone fusion polypeptide 1B7-G4-V0 processed; FIG. 49c is the amino acid sequence of growth hormone fusion polypeptide 1B7-G4-V1 unprocessed; FIG. 49d is the amino acid sequence of growth hormone fusion polypeptide 1B7-G4-V1 processed; FIG. 49e is the amino acid sequence of growth hormone fusion polypeptide 1B7-G4-V2 un processed; FIG. 49f is the amino acid sequence of growth hormone fusion polypeptide 1B7-G4-V2 processed;

FIG. 50a is the amino acid sequence of growth hormone fusion polypeptide 1B8-G1-V0; FIG. 50b is the amino acid sequence of growth hormone fusion polypeptide 1B8-G1-V1; FIG. 50c is the amino acid sequence of growth hormone fusion polypeptide 1B8-G1-V2; FIG. 50d is the amino acid sequence of growth hormone fusion polypeptide 1B8-G1-V3; FIG. 50e is the amino acid sequence of growth hormone fusion polypeptide 1B9-G1-V0; FIG. 50f is the amino acid sequence of growth hormone fusion polypeptide 1B9-G1-V1; FIG. 50g is the amino acid sequence of growth hormone fusion polypeptide 1B9-G1-V2; FIG. 50h is the amino acid sequence of growth hormone fusion polypeptide 1B9-G1-V3;

FIG. 51a is the amino acid sequence of growth hormone fusion polypeptide 1B8-G2-V0; FIG. 51 b is the amino acid sequence of growth hormone fusion polypeptide 1B8-G2-V1; FIG. 51c is the amino acid sequence of growth hormone fusion polypeptide 1B8-G2-V2; FIG. 51d is the amino acid sequence of growth hormone fusion polypeptide 1B8-G2-V3; FIG. 51e is the amino acid sequence of growth hormone fusion polypeptide 1B9-G2-V0; FIG. 51f is the amino acid sequence of growth hormone fusion polypeptide 1B9-G2-V1; FIG. 51g is the amino acid sequence of growth hormone fusion polypeptide 1B9-G2-V2; FIG. 51 h is the amino acid sequence of growth hormone fusion polypeptide 1B9-G2-V3;

FIG. 52a is the amino acid sequence of growth hormone fusion polypeptide 1B8-G3-V0; FIG. 52b is the amino acid sequence of growth hormone fusion polypeptide 1B8-G3-V1; FIG. 52c is the amino acid sequence of growth hormone fusion polypeptide 1B8-G3-V2; FIG. 52d is the amino acid sequence of growth hormone fusion polypeptide 1B8-G3-V3; FIG. 52e is the amino acid sequence of growth hormone fusion polypeptide 1B9-G3-V0; FIG. 52f is the amino acid sequence of growth hormone fusion polypeptide 1B9-G3-V1; FIG. 52g is the amino acid sequence of growth hormone fusion polypeptide 1B9-G3-V2; FIG. 52h is the amino acid sequence of growth hormone fusion polypeptide 1B9-G3-V3;

FIG. 53a is the amino acid sequence of growth hormone fusion polypeptide 1B8-G4-V0; FIG. 53b is the amino acid sequence of growth hormone fusion polypeptide 1B8-G4-V1; FIG. 53c is the amino acid sequence of growth hormone fusion polypeptide 1B8-G4-V2; FIG. 53d is the amino acid sequence of growth hormone fusion polypeptide 1B8-G4-V3; FIG. 53e is the amino acid sequence of growth hormone fusion polypeptide 1B9-G4-V0; FIG. 53f is the amino acid sequence of growth hormone fusion polypeptide 1B9-G4-V1; FIG. 53g is the amino acid sequence of growth hormone fusion polypeptide 1B9-G4-V2; FIG. 53h is the amino acid sequence of growth hormone fusion polypeptide 1B9-G4-V3;

FIG. 54a EPO-G1. 1 is the amino acid sequence for EPO-LI-EPOrEC: (EPO linked via a (G4S) 4 based glycolinker to EPOrEC) expressed in mammalian cells. (length=406aa without signal sequence). Signal sequence shown in bold; FIG. 54b EPO-G1. 2 is the amino acid sequence of EPO-L2-EPOrEC (EPO linked via a (G4S)3 based glycolinker to EPOrEC) expressed in mammalian cells. Sequence represents the full sequence expressed in a mammalian cell line along with the signal sequence. (length=401aa without signal sequence). Signal sequence shown in bold;

FIG. 55a EPO-G2. 1 is the amino acid sequence for EPO-L1-EPOrEC: (EPO linked via a (G4S) 4 based glycolinker to EPOrEC) expressed in mammalian cells. (length=406aa without signal sequence). Signal sequence shown in bold; FIG. 55b EPO-G2. 2 is the amino acid sequence of EPO-L2-EPOrEC (EPO linked via a (G4S)3 based glycolinker to EPOrEC) expressed in mammalian cells. Sequence represents the full sequence expressed in a mammalian cell line along with the signal sequence. (length=401aa without signal sequence). Signal sequence shown in bold;

FIG. 56a EPO-G3. 1 is the amino acid sequence for EPO-LI-EPOrEC: (EPO linked via a (G4S) 4 based glycolinker to EPOrEC) expressed in mammalian cells. (length=406aa without signal sequence). Signal sequence shown in bold; FIG. 56b EPO-G3. 2 is the amino acid sequence of EPO-L2-EPOrEC (EPO linked via a (G4S)3 based glycolinker to EPOrEC) expressed in mammalian cells. Sequence represents the full sequence expressed in a mammalian cell line along with the signal sequence. (length=401aa without signal sequence). Signal sequence shown in bold;

FIG. 57a EPO-G4. 1 is the amino acid sequence for EPO-LI-EPOrEC: (EPO linked via a (G4S) 4 based glycolinker to EPOrEC) expressed in mammalian cells. (length=406aa without signal sequence). Signal sequence shown in bold; FIG. 57b EPO-G4. 2 is the amino acid sequence of EPO-L2-EPOrEC (EPO linked via a (G4S)3 based glycolinker to EPOrEC) expressed in mammalian cells. Sequence represents the full sequence expressed in a mammalian cell line along with the signal sequence. (length=401aa without signal sequence). Signal sequence shown in bold;

FIG. 58a GCSF-G1. 1. amino acid sequence encoding GCSF-L6-GCSFrEC (1-3): contains GCSF linked via G4Sx6-based glycolinker to GCSF extracellular receptor domains 1-3 (Ig, BN and BC). amino acid sequence length=511aa (not including signal sequence);

FIG. 58b GCSF-G1. 2. amino acid sequence encoding GCSF-L8-GCSFrEC (1-3) expressed in a mammalian cell line: contains GCSF linked via G4Sx8-based glycolinker to GCSF extracellular receptor domains 1-3 (Ig, BN and BC). Signal sequence in bold and lower case; Amino acid sequence length=521 aa (not including signal sequence)

FIG. 58c GCSF-G1. 3. amino acid sequence encoding GCSF-L6-GCSFrEC (1-2): contains GCSF linked via G4Sx6-based glycolinker to GCSF extracellular receptor domains 1-2 (Ig and BN). Amino acid sequence length=404aa (not including signal sequence)

FIG. 58d GCSF-G1. 4. amino acid acid encoding GCSF-L6-GCSFrEC (2-3): contains GCSF linked via G4Sx6-based glycolinker to GCSF extracellular receptor domains 2-3 (BN and BC). *refers to stop codon. Signal sequence in bold and lower case; FIG. 58e GCSF-G1. 8b amino acid sequence length=416aa (not including signal sequence)

FIG. 58e GCSF-G1. 5. amino acid encoding GCSFrEC (1-3)-L6-GCSF: contains GCSFrEC (domains 1-3) linked via G4Sx6-based glycolinker to GCSF. Amino acid sequence length=511 as (not including signal sequence)

FIG. 58f GCSF-G1. 6. amino acid encoding GCSFrEC (2-3)-L6-GCSF expressed in a mammalian cell line: contains GCSFrEC (domains 2-3) linked via G4Sx6-based glycolinker to GCSF. Signal sequence in bold and lower case; amino acid sequence length=416aa (not including signal sequence)

FIG. 59a GCSF-G2. 1. amino acid sequence encoding GCSF-L6-GCSFrEC (1-3): contains GCSF linked via G4Sx6-based glycolinker to GCSF extracellular receptor domains 1-3 (Ig, BN and BC). amino acid sequence length=511aa (not including signal sequence);

FIG. 59b GCSF-G2. 2. amino acid sequence encoding GCSF-L8-GCSFrEC (1-3) expressed in a mammalian cell line: contains GCSF linked via G4Sx8-based glycolinker to GCSF extracellular receptor domains 1-3 (Ig, BN and BC). Signal sequence in bold and lower case; Amino acid sequence length=521aa (not including signal sequence)

FIG. 59c GCSF-G2. 3. amino acid sequence encoding GCSF-L6-GCSFrEC (1-2): contains GCSF linked via G4Sx6-based glycolinker to GCSF extracellular receptor domains 1-2 (Ig and BN). Amino acid sequence length=404aa (not including signal sequence)

FIG. 59d GCSF-G2. 4. amino acid acid encoding GCSF-L6-GCSFrEC (2-3): contains GCSF linked via G4Sx6-based glycolinker to GCSF extracellular receptor domains 2-3 (BN and BC). *refers to stop codon. Signal sequence in bold and lower case; Figure GCSF-G2. 8b amino acid sequence length=416aa (not including signal sequence)

FIG. 59e GCSF-G2. 5. amino acid encoding GCSFrEC (1-3)-L6-GCSF: contains GCSFrEC (domains 1-3) linked via G4Sx6-based glycolinker to GCSF. Amino acid sequence length=511aa (not including signal sequence)

FIG. 59f GCSF-G2. 6. amino acid encoding GCSFrEC (2-3)-L6-GCSF expressed in a mammalian cell line: contains GCSFrEC (domains 2-3) linked via G4Sx6-based glycolinker to GCSF. Signal sequence in bold and lower case; amino acid sequence length=416aa (not including signal sequence)

FIG. 60a GCSF-G3. 1. amino acid sequence encoding GCSF-L6-GCSFrEC (1-3): contains GCSF linked via G4Sx6-based glycolinker to GCSF extracellular receptor domains 1-3 (Ig, BN and BC). amino acid sequence length=511aa (not including signal sequence);

FIG. 60b GCSF-G3. 2. amino acid sequence encoding GCSF-L8-GCSFrEC (1-3) expressed in a mammalian cell line: contains GCSF linked via G4Sx8-based glycolinker to GCSF extracellular receptor domains 1-3 (Ig, BN and BC). Signal sequence in bold and lower case; Amino acid sequence length=521aa (not including signal sequence)

FIG. 60c GCSF-G3. 3. amino acid sequence encoding GCSF-L6-GCSFrEC (1-2): contains GCSF linked via G4Sx6-based glycolinker to GCSF extracellular receptor domains 1-2 (Ig and BN). Amino acid sequence length=404aa (not including signal sequence)

FIG. 60d GCSF-G3. 4. amino acid acid encoding GCSF-L6-GCSFrEC (2-3): contains GCSF linked via G4Sx6-based glycolinker to GCSF extracellular receptor domains 2-3 (BN and BC). *refers to stop codon. Signal sequence in bold and lower case; Figure GCSF-G3. 8b amino acid sequence length=416aa (not including signal sequence)

FIG. 60e GCSF-G3. 5. amino acid encoding GCSFrEC (1-3)-L6-GCSF: contains GCSFrEC (domains 1-3) linked via G4Sx6-based glycolinker to GCSF. Amino acid sequence length=511 aa (not including signal sequence)

FIG. 60f GCSF-G3. 6. amino acid encoding GCSFrEC (2-3)-L6-GCSF expressed in a mammalian cell line: contains GCSFrEC (domains 2-3) linked via G4Sx6-based glycolinker to GCSF. Signal sequence in bold and lower case; amino acid sequence length=416aa (not including signal sequence)

FIG. 61a GCSF-G4. 1. amino acid sequence encoding GCSF-L6-GCSFrEC (1-3): contains GCSF linked via G4Sx6-based glycolinker to GCSF extracellular receptor domains 1-3 (Ig, BN and BC). amino acid sequence length=511aa (not including signal sequence);

FIG. 61b GCSF-G4. 2. amino acid sequence encoding GCSF-L8-GCSFrEC (1-3) expressed in a mammalian cell line: contains GCSF linked via G4Sx8-based glycolinker to GCSF extracellular receptor domains 1-3 (Ig, BN and BC). Signal sequence in bold and lower case; Amino acid sequence length=521aa (not including signal sequence)

FIG. 61c GCSF-G4. 3. amino acid sequence encoding GCSF-L6-GCSFrEC (1-2): contains GCSF linked via G4Sx6-based glycolinker to GCSF extracellular receptor domains 1-2 (Ig and BN). Amino acid sequence length=404aa (not including signal sequence)

FIG. 61d GCSF-G4. 4. amino acid acid encoding GCSF-L6-GCSFrEC (2-3): contains GCSF linked via G4Sx6-based glycolinker to GCSF extracellular receptor domains 2-3 (BN and BC). *refers to stop codon. Signal sequence in bold and lower case; Figure GCSF-G4. 8b amino acid sequence length=416aa (not including signal sequence)

FIG. 61e GCSF-G4. 5. amino acid encoding GCSFrEC (1-3)-L6-GCSF: contains GCSFrEC (domains 1-3) linked via G4Sx6-based glycolinker to GCSF. Amino acid sequence length=511aa (not including signal sequence)

FIG. 61f GCSF-G4. 6. amino acid encoding GCSFrEC (2-3)-L6-GCSF expressed in a mammalian cell line: contains GCSFrEC (domains 2-3) linked via G4Sx6-based glycolinker to GCSF. Signal sequence in bold and lower case; amino acid sequence length=416aa (not including signal sequence)

FIG. 62a IFN-G1. 1 is the amino acid sequence of LR 7A1;

FIG. 62b IFN-G1. 2 is the amino acid sequence of LR 7B1;

FIG. 62c IFN-G1. 3 is the amino acid sequence of LR 7C1;

FIG. 62d IFN-G1. 4 is the amino acid sequence of LR 7D1;

FIG. 62e IFN-G1. 5 is the amino acid sequence of LR a7B1;

FIG. 63a IFN-G2. 1 is the amino acid sequence of LR 7A1;

FIG. 63b IFN-G2. 2 is the amino acid sequence of LR 7B1;

FIG. 63c IFN-G2. 3 is the amino acid sequence of LR 7C1;

FIG. 63d IFN-G2. 4 is the amino acid sequence of LR 7D1;

FIG. 63e IFN-G2. 5 is the amino acid sequence of LR a7B1;

FIG. 64a IFN-G3. 1 is the amino acid sequence of LR 7A1;

FIG. 64b IFN-G3. 2 is the amino acid sequence of LR 7B1;

FIG. 64c IFN-G3. 3 is the amino acid sequence of LR 7C1;

FIG. 64d IFN-G3. 4 is the amino acid sequence of LR 7D1;

FIG. 64e IFN-G3. 5 is the amino acid sequence of LR a7B1;

FIG. 65a IFN-G4. 1 is the amino acid sequence of LR 7A1;

FIG. 65b IFN-G4. 2 is the amino acid sequence of LR 7B1;

FIG. 65c IFN-G4. 3 is the amino acid sequence of LR 7C1;

FIG. 65d IFN-G4. 4 is the amino acid sequence of LR 7D1;

FIG. 65e IFN-G4. 5 is the amino acid sequence of LR a7B1;

FIG. 66a IGF1-G1. 1 is the amino acid sequence of LR5A1;

FIG. 66b IGF1-G1. 2 is the amino acid sequence of LR5B1;

FIG. 66c IGF1-G1. 3 is the amino acid sequence of LR5C1;

FIG. 66d IGF1-G1. 4 is the amino acid sequence of LR5D1;

FIG. 66e IGF1-G1. 5 is the amino acid sequence of LR5E1;

FIG. 66f IGF1-G1. 6 is the amino acid sequence of LR5F1;

FIG. 67a IGF1-G2. 1 is the amino acid sequence of LR5A1;

FIG. 67b IGF1-G2. 2 is the amino acid sequence of LR5B1;

FIG. 67c IGF1-G2. 3 is the amino acid sequence of LR5C1;

FIG. 67d IGF1-G2. 4 is the amino acid sequence of LR5D1;

FIG. 67e IGF1-G2. 5 is the amino acid sequence of LR5E1;

FIG. 67f IGF1-G2. 6 is the amino acid sequence of LR5F1;

FIG. 68a IGF1-G3. 1 is the amino acid sequence of LR5A1;

FIG. 68b IGF1-G3. 2 is the amino acid sequence of LR5B1;

FIG. 68c IGF1-G3. 3 is the amino acid sequence of LR5C1;

FIG. 68d IGF1-G3. 4 is the amino acid sequence of LR5D1;

FIG. 68e IGF1-G3. 5 is the amino acid sequence of LR5E1;

FIG. 68f IGF1-G3. 6 is the amino acid sequence of LR5F1;

FIG. 69a IGF1-G4. 1 is the amino acid sequence of LR5A1;

FIG. 69b IGF1-G4. 2 is the amino acid sequence of LR5B1;

FIG. 69c IGF1-G4. 3 is the amino acid sequence of LR5C1;

FIG. 69d IGF1-G4. 4 is the amino acid sequence of LR5D1;

FIG. 69e IGF1-G4. 5 is the amino acid sequence of LR5E1;

FIG. 69f IGF1-G4. 6 is the amino acid sequence of LR5F1;

FIG. 70a IL2-G1. 1 is the amino acid sequence of LR 6A1;

FIG. 70b IL2-G1. 2 is the amino acid sequence of LR 6B1;

FIG. 70c IL2-G1. 3 is the amino acid sequence of LR 6C1;

FIG. 70d IL2-G1. 4 is the amino acid sequence of LR 6D1;

FIG. 70e IL2-G1. 5 is the amino acid sequence of LR 6E1;

FIG. 70f IL2-G1. 6 is the amino acid sequence of LR 6E2;

FIG. 70g IL2-G1. 7 is the amino acid sequence of LR 6F1;

FIG. 70h IL2-G1. 8 is the amino acid sequence of LR fusion IL7ss-IL7-(G₄S)₄-IL7Ralpha;

FIG. 70i IL2-G1. 9 is the amino acid sequence of LR fusion IL7ss-IL7-(G₄S)₅-IL2Rgamma;

FIG. 70j IL2-G1. 10 is the amino acid sequence of LR fusion IL7Rass-IL7Ralpha-(G4S)₄-I L7;

FIG. 70k IL2-G1. 11 is the amino acid sequence of LR fusion IL2gss-IL2gamma-(G₄S)₅-IL7;

FIG. 701 IL2-G1. 12 is the amino acid sequence of LR fusion IL7-(G₄S)₄-IL7Ralpha;

FIG. 70m IL2-G1. 13 is the amino sequence of LR fusion IL7-(G₄S)₅-IL2Rgamma;

FIG. 70n IL2-G1. 14 is the amino sequence of LR fusion IL7RaIpha-(G₄S)₄-IL7;

FIG. 70o IL2-G1. 15 is the amino acid sequence of LR fusion IL2gamma-(G₄S)₅-IL7;

FIG. 70p IL2-G1. 16 is the amino acid sequence of LR a7B1

FIG. 71a IL2-G2. 1 is the amino acid sequence of LR 6A1;

FIG. 71b IL2-G2. 2 is the amino acid sequence of LR 6B1;

FIG. 71c IL2-G2. 3 is the amino acid sequence of LR 6C1;

FIG. 71d IL2-G2. 4 is the amino acid sequence of LR 6D1;

FIG. 71e IL2-G2. 5 is the amino acid sequence of LR 6E1;

FIG. 71f IL2-G2. 6 is the amino acid sequence of LR 6E2;

FIG. 71g IL2-G2. 7 is the amino acid sequence of LR 6F1;

FIG. 71h IL2-G2. 8 is the amino acid sequence of LR fusion IL7ss-IL7-(G₄S)₄-IL7Ralpha;

FIG. 71i IL2-G2. 9 is the amino acid sequence of LR fusion IL7ss-IL7-(G₄S)₅-IL2Rgamma;

FIG. 71j IL2-G2. 10 is the amino acid sequence of LR fusion IL7Rass-IL7RaIpha-(G₄S)₄-I L7;

FIG. 71k IL2-G2. 11 is the amino acid sequence of LR fusion IL2gss-IL2gamma-(G₄S)₅-IL7;

FIG. 71l IL2-G2. 12 is the amino acid sequence of LR fusion IL7-(G₄S)₄-IL7RaIpha;

FIG. 71m IL2-G2. 13 is the amino sequence of LR fusion IL7-(G₄S)₅-IL2Rgamma;

FIG. 71 n 1L2-G2. 14 is the amino sequence of LR fusion IL7RaIpha-(G₄S)₄-IL7;

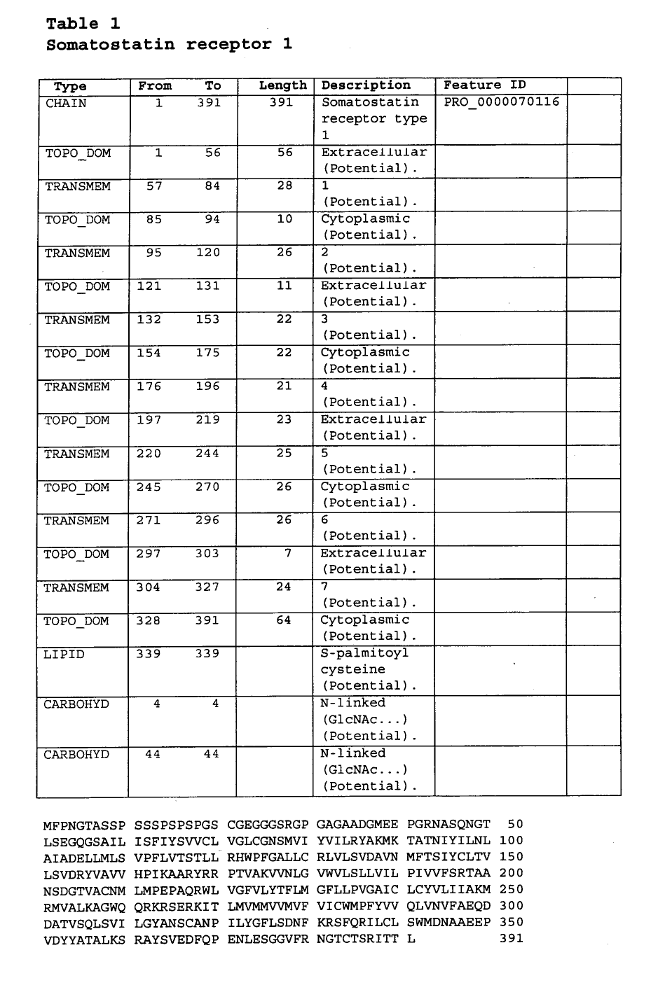

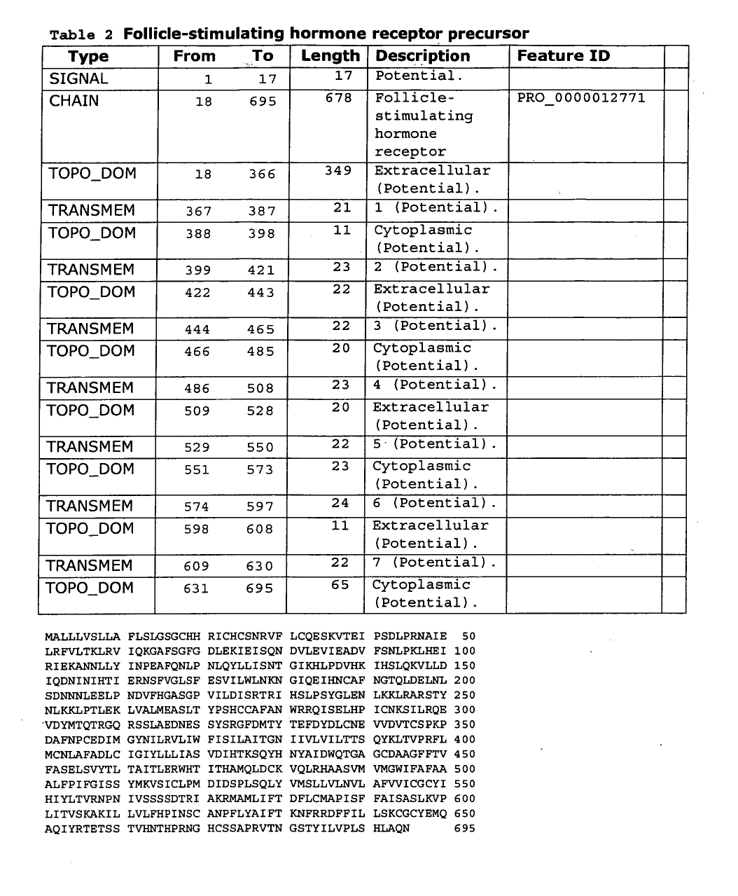

FIG. 710 IL2-G2. 15 is the amino acid sequence of LR fusion IL2gamma-(G₄S)₅-IL7;

FIG. 71p 1L2-G2. 16 is the amino acid sequence of LR a7B1

FIG. 72a 1L2-G3. 1 is the amino acid sequence of LR 6A1;

FIG. 72b IL2-G3. 2 is the amino acid sequence of LR 6B1;

FIG. 72c IL2-G3. 3 is the amino acid sequence of LR 6C1;

FIG. 72d IL2-G3. 4 is the amino acid sequence of LR 6D1;

FIG. 72e IL2-G3. 5 is the amino acid sequence of LR 6E1;

FIG. 72f 1L2-G3. 6 is the amino acid sequence of LR 6E2;

FIG. 72g IL2-G3. 7 is the amino acid sequence of LR 6F1;

FIG. 72h 11_2-G3. 8 is the amino acid sequence of LR fusion IL7ss-IL7-(G₄S)₄-IL7Ralpha;

FIG. 72i IL2-G3. 9 is the amino acid sequence of LR fusion IL7ss-IL7-(G₄S)₅-IL2Rgamma;

FIG. 72j IL2-G3. 10 is the amino acid sequence of LR fusion IL7Rass-IL7Ralpha-(G₄S)₄-IL7;

FIG. 72k IL2-G3. 11 is the amino acid sequence of LR fusion IL2gss-IL2gamma-(G₄S)₅-IL7;

FIG. 72l IL2-G3. 12 is the amino acid sequence of LR fusion IL7-(G₄S)₄-IL7Ralpha;

FIG. 72m IL2-G3. 13 is the amino sequence of LR fusion IL7-(G₄S)₅-IL2Rgamma;

FIG. 72n IL2-G3. 14 is the amino sequence of LR fusion IL7Ralpha-(G₄S)₄-IL7;

FIG. 72o IL2-G3. 15 is the amino acid sequence of LR fusion IL2gamma-(G₄S)₅-IL7;

FIG. 72p IL2-G3. 16 is the amino acid sequence of LR a7B1

FIG. 73a IL2-G4. 1 is the amino acid sequence of LR 6A1;

FIG. 73b IL2-G4. 2 is the amino acid sequence of LR 6B1;

FIG. 73c IL2-G4. 3 is the amino acid sequence of LR 6C1;

FIG. 73d IL2-G4. 4 is the amino acid sequence of LR 601;

FIG. 73e IL2-G4. 5 is the amino acid sequence of LR 6E1;

FIG. 73f IL2-G4. 6 is the amino acid sequence of LR 6E2;

FIG. 73g IL2-G4. 7 is the amino acid sequence of LR 6F1;

FIG. 73h IL2-G4. 8 is the amino acid sequence of LR fusion IL7ss-IL7-(G₄S)₄-IL7Ralpha;

FIG. 73i IL2-G4. 9 is the amino acid sequence of LR fusion IL7ss-IL7-(G₄S)₅-IL2Rgamma;

FIG. 73j IL2-G4. 10 is the amino acid sequence of LR fusion IL7Rass-IL7RaIpha-(G₄S)₄-IL7;

FIG. 73k IL2-G4. 11 is the amino acid sequence of LR fusion IL2gss-IL2gamma-(G₄S)₅-IL7;

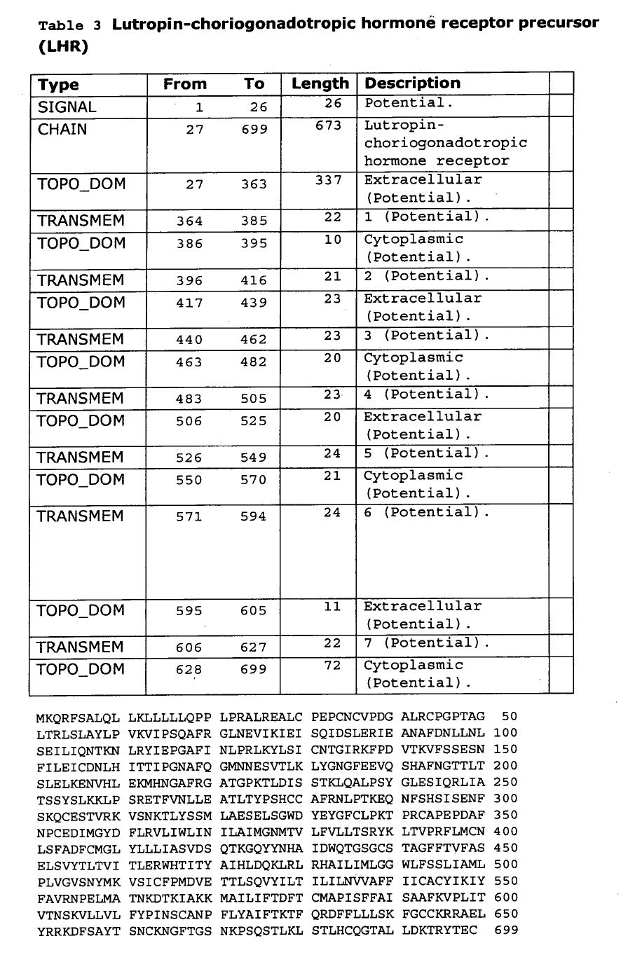

FIG. 731 IL2-G4. 12 is the amino acid sequence of LR fusion IL7-(G₄S)₄-IL7Ralpha;

FIG. 73m 1L2-G4. 13 is the amino sequence of LR fusion IL7-(G₄S)₅-IL2Rgamma;

FIG. 73n IL2-G4. 14 is the amino sequence of LR fusion IL7Ralpha-(G₄S)₄-IL7;

FIG. 73o IL2-G4. 15 is the amino acid sequence of LR fusion IL2gamma-(G₄S)₅-IL7;

FIG. 73p IL2-G4. 16 is the amino acid sequence of LR a7B1

FIG. 74a LEPTIN-G1. 1 is the amino acid sequence of LR 2A1;

FIG. 74b LEPTIN-G1. 2 is the amino acid sequence of LR 2A1 adapted for bacterial expression;

FIG. 74c LEPT1N-G1. 3 is the amino acid sequence of LR 2B1;

FIG. 74d LEPTIN-G1. 4 is the amino acid sequence of LR 2D1;

FIG. 74e LEPTIN-G1. 5 is the amino acid sequence of LR 2E1;

FIG. 74f LEPTIN-G1. 6 is the amino acid sequence of LR 2F1;

FIG. 74g LEPTIN-G1. 7 is the amino acid sequence of LR 2G1;

FIG. 74h LEPT1N-G1. 8 is the amino acid sequence of LR 2H1;

FIG. 74i LEPTIN-G1. 9 is the amino acid sequence of LR 2I1;

FIG. 74j LEPTIN-G1. 10 is the amino acid sequence of LR 2J1;

FIG. 74k LEPTIN-G1. 11 is the amino acid sequence of LR 2K1;

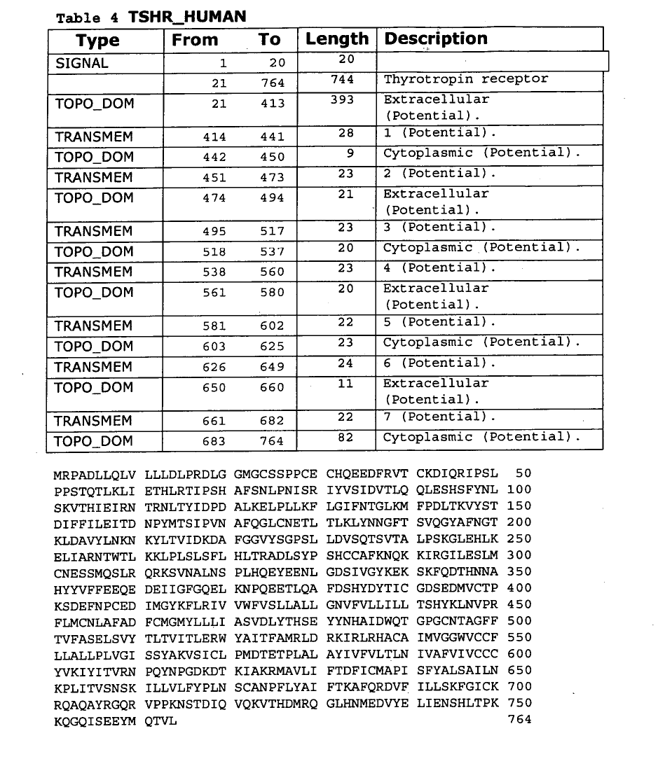

FIG. 741 LEPTIN-G1. 12 is the amino acid sequence of LR 2L1;

FIG. 74m LEPT1N-G1. 13 is the amino acid sequence of LR 2M1;

FIG. 75a LEPTIN-G2. 1 is the amino acid sequence of LR 2A1;

FIG. 75b LEPTIN-G2. 2 is the amino acid sequence of LR 2A1 adapted for bacterial expression;

FIG. 75c LEPTIN-G2. 3 is the amino acid sequence of LR 2B1;

FIG. 75d LEPTIN-G2. 4 is the amino acid sequence of LR 2D1;

FIG. 75e LEPTIN-G2. 5 is the amino acid sequence of LR 2E1;

FIG. 75 LEPTIN-G2. 6 is the amino acid sequence of LR 2F1;

FIG. 75g LEPTIN-G2. 7 is the amino acid sequence of LR 2G1;

FIG. 75h LEPTIN-G2. 8 is the amino acid sequence of LR 2H1;

FIG. 75i LEPTIN-G2. 9 is the amino acid sequence of LR 2I1;

FIG. 75j LEPTIN-G2. 10 is the amino acid sequence of LR 2J1;

FIG. 75k LEPTIN-G2. 11 is the amino acid sequence of LR 2K1;

FIG. 75I LEPTIN-G2. 12 is the amino acid sequence of LR 2L1;

FIG. 75m LEPTIN-G2. 13 is the amino acid sequence of LR 2M1;

FIG. 76a LEPTIN-G3. 1 is the amino acid sequence of LR 2A1;

FIG. 76b LEPTIN-G3. 2 is the amino acid sequence of LR 2A1 adapted for bacterial expression;

FIG. 76c LEPTIN-G3. 3 is the amino acid sequence of LR 2B1;

FIG. 76d LEPTIN-G3. 4 is the amino acid sequence of LR 2D1;

FIG. 76e LEPTIN-G3. 5 is the amino acid sequence of LR 2E1;

FIG. 76f LEPTIN-G3. 6 is the amino acid sequence of LR 2F1;

FIG. 76g LEPTIN-G3. 7 is the amino acid sequence of LR 2G1;

FIG. 76h LEPTIN-G3. 8 is the amino acid sequence of LR 2H1;

FIG. 76i LEPTIN-G3. 9 is the amino acid sequence of LR 2I1;

FIG. 76j LEPTIN-G3. 10 is the amino acid sequence of LR 2J1;

FIG. 76k LEPTIN-G3. 11 is the amino acid sequence of LR 2K1;

FIG. 76l LEPTIN-G3. 12 is the amino acid sequence of LR 2L1;

FIG. 76m LEPTIN-G3. 13 is the amino acid sequence of LR 2M1;

FIG. 77a LEPTIN-G4. 1 is the amino acid sequence of LR 2A1;

FIG. 77b LEPTIN-G4. 2 is the amino acid sequence of LR 2A1 adapted for bacterial expression;

FIG. 77c LEPTIN-G4. 3 is the amino acid sequence of LR 2B1;

FIG. 77d LEPTIN-G4. 4 is the amino acid sequence of LR 2D1;

FIG. 77e LEPTIN-G4. 5 is the amino acid sequence of LR 2E1;

FIG. 77f LEPTIN-G4. 6 is the amino acid sequence of LR 2F1;

FIG. 77g LEPTIN-G4. 7 is the amino acid sequence of LR 2G1;

FIG. 77h LEPTIN-G4. 8 is the amino acid sequence of LR 2H1;

FIG. 77i LEPTIN-G4. 9 is the amino acid sequence of LR 2I1;

FIG. 77j LEPTIN-G4. 10 is the amino acid sequence of LR 2J1;

FIG. 77k LEPTIN-G4. 11 is the amino acid sequence of LR 2K1;

FIG. 77l LEPTIN-G4. 12 is the amino acid sequence of LR 2L1;

FIG. 77m LEPTIN-G4. 13 is the amino acid sequence of LR 2M1;

FIG. 78a PRL-G1. 1 is the amino acid sequence of 8B7v2: PRL linked via a G4Sx6-based glycolinker to PRLRext amino acid Protein sequence: 439 amino acids (not including signal sequence);

FIG. 78b PRL-G1. 2 is the amino acid sequence of 8B8v2: PRL (G129Rmutation) linked via G4Sx6 based glycolinker to PRLRext, signal sequence shown in bold. G129R mutation highlighted; (439 amino acids not including signal sequence).

FIG. 78c PRL-G1. 3 is the amino acid sequence of 8B9v2: Consists of PRL (Deleted N-terminal residues 1-9/C11S/G129R mutations) linked via a G4S x6-based glycolinker to PRLRext. Signal sequence is shown in bold. C11S and G129R mutations highlighted; 430 amino acids (not including signal sequence).

FIG. 79a PRL-G2. 1 is the amino acid sequence of 8B7v2: PRL linked via a G4Sx6-based glycolinker to PRLRext amino acid Protein sequence: 439 amino acids (not including signal sequence);

FIG. 79b PRL-G2. 2 is the amino acid sequence of 8B8v2: PRL (G129Rmutation) linked via G4Sx6 based glycolinker to PRLRext, signal sequence shown in bold. G129R mutation highlighted; (439 amino acids not including signal sequence).

FIG. 79c PRL-G2. 3 is the amino acid sequence of 8B9v2: Consists of PRL (Deleted N-terminal residues 1-9/C11S/G129R mutations) linked via a G4S x6-based glycolinker to PRLRext. Signal sequence is shown in bold. C11S and G129R mutations highlighted; 430 amino acids (not including signal sequence).

FIG. 80a PRL-G3. 1 is the amino acid sequence of 8B7v2: PRL linked via a G4Sx6-based glycolinker to PRLRext amino acid Protein sequence: 439 amino acids (not including signal sequence);

FIG. 80b PRL-G3. 2 is the amino acid sequence of 8B8v2: PRL (G129Rmutation) linked via G4Sx6 based glycolinker to PRLRext, signal sequence shown in bold. G129R mutation highlighted; (439 amino acids not including signal sequence).

FIG. 80c PRL-G3. 3 is the amino acid sequence of 8B9v2: Consists of PRL (Deleted N-terminal residues 1-9/C11S/G129R mutations) linked via a G4S x6-based glycolinker to PRLRext. Signal sequence is shown in bold. C11S and G129R mutations highlighted; 430 amino acids (not including signal sequence).

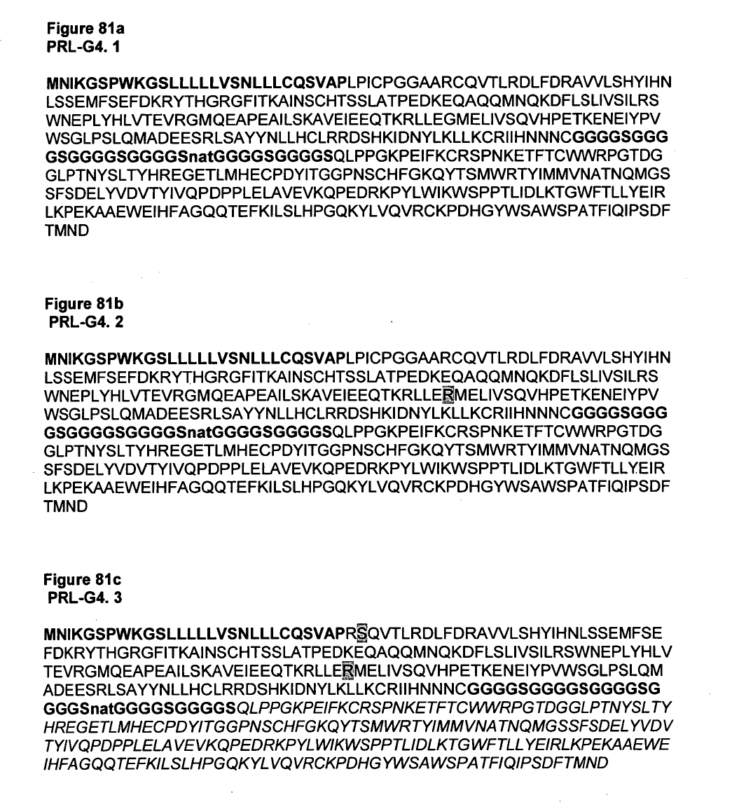

FIG. 81a PRL-G4. 1 is the amino acid sequence of 8B7v2: PRL linked via a G4Sx6-based glycolinker to PRLRext amino acid Protein sequence: 439 amino acids (not including signal sequence);

FIG. 81b PRL-G4. 2 is the amino acid sequence of 8B8v2: PRL (G129Rmutation) linked via G4Sx6 based glycolinker to PRLRext, signal sequence shown in bold. G129R mutation highlighted; (439 amino acids not including signal sequence).

FIG. 81c PRL-G4. 3 is the amino acid sequence of 8B9v2: Consists of PRL (Deleted N-terminal residues 1-9/C11S/G129R mutations) linked via a G4S x6-based glycolinker to PRLRext. Signal sequence is shown in bold. C11S and G129R mutations highlighted; 430 amino acids (not including signal sequence).

Table 1 illustrates the domains in full length somatostatin 1 receptor and the full length amino acid sequence;

Table 2 illustrates the domains in full length FSH receptor and the full length amino acid sequence;

Table 3 illustrates the domains in full length LH receptor and the full length amino acid sequence;

Table 4 illustrates the domains in full length TSH receptor and the full length amino acid sequence; and

Table 5 illustrates growth hormone tandem construct nomenclature referred to in the examples and figures.

Materials and Methods

Immunological Testing

Immunoassays that measure the binding of fusion protein or receptor to polyclonal and monoclonal antibodies are known in the art. Commercially available antibodies are available to detect the fusion protein or receptor in samples and also for use in competitive inhibition studies.

Recombinant Production of Fusion Proteins

The components of the fusion proteins were generated by PCR using primers designed to anneal to the ligand or receptor and to introduce suitable restriction sites for cloning into the target vector. The template for the PCR comprised the target gene and was obtained from IMAGE clones, cDNA libraries or from custom synthesised genes. Once the ligand and receptor genes with the appropriate flanking restriction sites had been synthesised, these were then ligated either side of the linker region in the target vector. The construct was then modified to contain the correct linker without flanking restriction sites by the insertion of a custom synthesised length of DNA between two unique restriction sites either side of the linker region, by mutation of the linker region by ssDNA modification techniques, by insertion of a primer duplex/multiplex between suitable restriction sites or by PCR modification.

Alternatively, the linker with flanking sequence, designed to anneal to the ligand or receptor domains of choice, was initially synthesised by creating an oligonucleotide duplex and this processed to generate double-stranded DNA. PCRs were then performed using the linker sequence as a “megaprimer”, primers designed against the opposite ends of the ligand and receptor to which the “megaprimer” anneals to and with the ligand and receptor as the templates. The terminal primers were designed with suitable restriction sites for ligation into the expression vector of choice.

Expression and Purification of Glycosylated Fusion Proteins

Expression was carried out in a suitable system (e.g. mammalian CHO cells,) and this was dependant on the vector into which the LR-fusion gene was generated. Expression was then analysed using a variety of methods which could include one or more of SDS-PAGE, Native PAGE, western blotting, ELISA.

Once a suitable level of expression was achieved the LR-fusions were expressed at a larger scale to produce enough protein for purification and subsequent analysis.

Purification was carried out using a suitable combination of one or more chromatographic procedures such as ion exchange chromatography, hydrophobic interaction chromatography, ammonium sulphate precipitation, gel filtration, size exclusion and/or affinity chromatography (using nickel/cobalt-resin, antibody-immobilised resin and/or ligand/receptor-immobilised resin).

Purified protein was analysed using a variety of methods which could include one or more of Bradford's assay, SDS-PAGE, Native PAGE, western blotting, ELISA.

Characterisation of Glycosylated Fusion Proteins

Denaturing PAGE, native PAGE gels and western blotting were used to analyse the fusion polypeptides and western blotting performed with antibodies non-conformationally sensitive to the fusion protein. Native solution state molecular weight information can be obtained from techniques such as size exclusion chromatography using a Superose G200 analytical column and analytical ultracentrifugation.

Construction of Growth Hormone Tandem Molecules

The method utilises glycosylated linkers (consisting of variable repeats of Gly4Ser) to increase the molecular weight of the protein. N-linked glycosylation recognition sequences are inserted into these linker regions of tandem GH molecules (Sequence is composed of either Asn-X-Ser or Asn-X-Thr, wherein X can be any amino acid except Pro).

All molecules were gene synthesized and ligated into the mammalian expression vector pSecTag-link. Protein is expressed as a secreted product under the control of the growth hormone secretion signal.

Mammalian Stable Expression

A mammalian expression system has been established using a modification of the invitrogen vector pSecTag-V5/FRT-Hist

Invitrogen's Flp-In System

This system allows for the rapid generation of stable clones into specific sites within the host genome for high expression. This can be used with either secreted or cytoplasmic expressed proteins. Flp-In host cell lines (flp-In CHO) have a single Flp recombinase target (FRT) site located at a transcriptionally active genomic locus

Stable cell lines are generated by co-transfection of vector (Containing FRT target site) and pOG44 (a [plasmid that transiently expresses flp recombinase) into Flp-In cell line. Selection is with Hygromycin B. There is no need for clonal selection since integration of DNA is directed. Culturing Flp-In Cell lines: followed manufactures instruction using basic cell culture techniques.

Stable Transfection of CHO FIp-In Cells using Fugene-6

The day before transfection CHO FIp-In cells were seeded at 6×10E5 per 100 mm petri dish in a total volume of 10 ml of Hams F12 media containing 10% (v/v) Fetal Calf Serum, 1% Penicillin/streptomycin and 4 mM L-glutamine. The next day added 570 μl of serum free media (containing no antibiotics) to a 1.5 ml polypropylene tube. 30 μl of fugene-6 was then added and mixed by gentle rolling. A separate mix of plasmids was set up for each transfection which combined 2 μg plasmid of interest with 18 μg pOG44 (plasmid contains recombinase enzyme necessary for correct integration of plasmid into host genome). Control plate received no plasmid. This was mixed with fugene-6 by gentle rolling, left @ RmT for 15 minutes, then applied drop-wise to the surface of the each petri dish containing CHO FIp-In cells in F12 media+10% FCS. The plates were gently rolled to ensure good mixing and left for 24 hrs @ 37° C./5% CO2. The next day media was exchanged for selective media containing hygromycin B @ 600 ug/ml. Cells were routinely kept at 60% confluency or less. Cells were left to grow in the presence of 600 ug/ml hygromycin B until control plate cells (non transfected cells) had died (i.e. no hygromycin resistance).

Testing Expression from Stable CHO Cell Lines

Confluent CHO FIp-In cell lines expressing the protein of interest were grown in 75 cm2 flasks for approximately 3-4 days in serum free media, at which point samples were taken and mixed with an equal volume of Laemmli loading buffer in the presence or absence of 25 mM DTT and heated at 65 C for 15 minutes. Samples were analysed by SDS-PAGE and transferred to a PVDF membrane. After blocking in 5% (w/v) Milk protein in PBS-0.05% (v/v) Tween 20, sample detection was carried out using a specific anti-GH antibody together with a Horse Radish Peroxidase (HRP) conjugated secondary antibody. Visualisation was by chemiluminesence on photographic film using an HRP detection kit.

Testing Expression from Transient Tansfections

CHO Flp-In cells were seeded at 0.25×10E6 cells per well of a 6 well plate in a total volume of 2 ml media (DMEM, F12, 10% FCS+P/S+L-glutamine+Zeocin). Cells were left to grow o/n. Cells were then transfected using either TranslT-CHO Reagent (Mirus) or fugene-6 at the specified reagent ratios stated in table 1. Briefly, if using TransiT reagent, 200 ul of Serum free media (OPTI MEM) was added to a 1.5 ml eppendorff per transfection followed by 2 ug DNA. The tubes were left for 15 minutes at RmT. 1 ul of CHO Mojo Reagent was then added, mixed and left for a further 15 minutes. Media was changed to serum free and the transfection mix pippetted dropwise onto the surface of the appropriate well. Briefly, if using Fugene-6 reagent, 94 ul of Serum free media (OPTI MEM) was added to a 1.5 ml eppendorff per transfection followed by 2 ug DNA. The tubes were left for 15 minutes at RmT. Trasfection mix was then pippetted drop wise onto the surface of the appropriate well containing serum free media. All plate were left @ 37 degC./5% CO2 for 2-3 days. If required, samples were concentrated using acetone precipitation.

1. A fusion polypeptide comprising a first polypeptide which is a cytokine and a second polypeptide which is the extracellular domain of the cognate receptor to which the cytokine binds, wherein the cytokine polypeptide and extracellular domain of the cognate receptor are linked indirectly by a peptide linker, and wherein said peptide linker is modified to include at least one motif for the addition of at least one sugar moiety.

2-19. (canceled)

20. A fusion polypeptide according to claim 1 wherein said peptide linker molecule comprises one copy of the glycosylation motif Asn-Xaa-Ser or Asn-Xaa-Thr where X is any amino acid except proline.

21. A fusion polypeptide according to claim 20 wherein said peptide linker molecule comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr.

22. A fusion polypeptide according to claim 21 wherein said peptide linker comprises at least one copy of an amino acid motif selected from the group consisting of:
Asn₁-Xaa₂-Ser₃ Xaa₄ Xaa₅ wherein Xaa₂ is any amino acid except proline;
Xaa₁ Asn₂-Xaa₃-Ser₄ Xaa₅ wherein Xaa₃ is any amino acid except proline;
Xaa₁ Xaa₂ Asn₃-Xaa₄-Ser₅ wherein Xaa₄ is any amino acid except proline;
Asn₁-Xaa₂-Thr₃ Xaa₄ Xaa₅ wherein Xaa₂ is any amino acid except proline;
Xaa₁ Asn₂-Xaa₃-Thr₄ Xaa₅ wherein Xaa₃ is any amino acid except proline; and
Xaa₁ Xaa₂ Asn₃-Xaa₄-Thr₅ wherein Xaa₄ is any amino acid except proline.

23. A fusion polypeptide according to claim 21 wherein said peptide linker comprises at least one copy of a motif selected from the group consisting of:
Asn₁-Xaa₂-Ser₃ Gly₄ Ser₅ wherein Xaa₂ is any amino acid except proline;
Gly₁ Asn₂-Xaa₃-Ser₄ Ser₅ wherein Xaa₃ is any amino acid except proline;
Gly₁ Gly₂ Asn₃-Xaa₄-Ser₅ wherein Xaa₄ is any amino acid except proline;
Asn₁-Xaa₂-Thr₃ Gly₄ Ser₅ wherein Xaa₂ is any amino acid except proline;
Gly₁ Asn₂-Xaa₃-Thr₄ Ser₅ wherein Xaa₃ is any amino acid except proline; and
Gly₁ Gly₂ Asn₃-Xaa₄-Thr₅ wherein Xaa₄ is any amino acid except proline.

24. A fusion polypeptide according to claim 21 wherein said peptide linker comprises at least one copy of a motif selected from the group consisting of:
Asn₁-Xaa₂-Ser₃ Ser₄ Gly₅ wherein Xaa₂ is any amino acid except proline;
Ser₁ Asn₂-Xaa₃-Ser₄ Gly₅ wherein Xaa₃ is any amino acid except proline;
Ser₁ Ser₂ Asn₃-Xaa₄-Ser₅ wherein Xaa₄ is any amino acid except proline;
Asn₁-Xaa₂-Thr₃ Ser₄ Gly₅ wherein Xaa₂ is any amino acid except proline;
Ser₁ Asn₂-Xaa₃-Thr₄ Gly₅ wherein Xaa₃ is any amino acid except proline; and
Ser₁ Ser₂ Asn₃-Xaa₄-Thr₅ wherein Xaa₄ is any amino acid except proline.

25. A fusion polypeptide according to claim 21 wherein said peptide linker molecule comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr and at least one copy of the motif (Gly Gly Gly Gly Ser) wherein said peptide linker is 5-50 amino acids.

26. A fusion polypeptide according to claim 21 wherein said peptide linker comprises at least one copy of the motif (Xaa₁ Xaa₂ Xaa₃ Xaa₄ Xaa₅) wherein said motif comprises the glycosylation motif Asn-Xaa-Ser or Asn-Xaa-Thr and a copy of the motif (Ser Ser Ser Ser Gly) wherein said peptide linker is 5-50 amino acids.

27. A nucleic acid molecule that encodes a polypeptide according to claim 1.

28. A vector comprising the nucleic acid molecule according to claim 27.

29. An isolated host cell transfected or transformed with a nucleic acid molecule or vector according to claim 28.

30. A pharmaceutical composition comprising a polypeptide according to claim 1 and an excipient or carrier.

31-69. (canceled)

70. A fusion polypeptide according to claim 20 wherein the cytokine is selected from the group consisting of: growth hormone, erythropoietin, granulocyte colony stimulating hormone, a type 1 interferon, interleukin 2, leptin and prolactin.

71. A fusion polypeptide according to claim 70 wherein said polypeptide comprises an amino acid sequence selected from the group consisting of the sequences represented in FIGS. 46a, 46b, 46c, 46d, 46e, 46f, 47a, 47b, 47c, 47d, 47e 47f, 48a, 48b, 48c, 48d, 48e, 48f, 49a, 49b, 49c, 49d, 49e, 49f, 50a, 50b, 50c, 50d, 50h, 51a, 51b, 51c, 51d, 51e, 51f, 51h, 52a, 52b, 52c, 52d, 52e, 52f, 52h, 53a, 53b, 53c, 53d, 53e, 53f amd 53h.

72. A fusion polypeptide according to claim 70 wherein said polypeptide comprises an amino acid sequence selected from the group consisting of the sequences represented in FIGS. 54a, 54b, 55a, 55b, 56a, 56b, 57a and 57b.

73. A fusion polypeptide according to claim 70 wherein said polypeptide comprises an amino acid sequence selected from the group consisting of the sequences represented in FIGS. 58a, 58b, 58c, 58d, 58e, 58f, 59a, 59a, 59b, 59c, 59d, 59e, 59f, 60a, 60b, 60c, 60d, 60e, 60f, 61a, 61b, 61c, 61d, 61e and 61f.

74. A fusion polypeptide according to claim 70 wherein said polypeptide comprises an amino acid sequence selected from the group consisting of the sequences represented in FIGS. 62a, 62b, 62c, 62d, 62e, 63a, 63b, 63c, 63d, 63e, 64a, 64b, 64c, 64d, 64e, 65a, 65b, 65c, 65d, 65e, 66a, 66b, 66c, 66d, 66e and 66f.

75. A fusion polypeptide according to claim 70 wherein said polypeptide comprises an amino acid sequence selected from the group consisting of the sequences represented in FIGS. 70a, 70b, 70c, 70d, 70e, 70f, 70g, 70h, 70i, 70j, 70k, 70m, 70n, 70o, 70p, 71a, 71b, 71c, 71d, 71e, 71f, 71g, 71h, 71i, 71j, 71k, 71m, 71n, 71o, 71p, 72a, 72b, 72c, 72d, 72e, 72f, 72g, 72h, 72i, 72j, 72k, 72m, 72n, 72o, 72p, 73a, 73b, 73c, 73d, 73e, 73f, 73g, 73h, 73i, 73j, 73k, 73m, 73n, 73o and 73p.

76. A fusion polypeptide according to claim 70 wherein said polypeptide comprises an amino acid sequence selected from the group consisting of the sequences represented in FIGS. 74a, 74b, 74c, 74d, 74e, 74f, 74g, 74h, 74i, 74j, 74k, 74m, 75a, 75b, 75c, 75d, 75e, 75f, 75g, 75h, 75i, 75j, 75k, 75m, 76a, 76b, 76c, 76d, 76e, 76f, 76g, 76h, 76i, 76j, 76k, 76m, 77a, 77b, 77c, 77d, 77e, 77f, 77g, 77h, 77i, 77j, 77k and 77m.

77. A fusion polypeptide according to claim 70 wherein said polypeptide comprises an amino acid sequence selected from the group consisting of the sequences represented in FIGS. 78a, 78b, 78c, 79a, 79b, 79c, 80a, 80b, 80c, 81a, 81b and 81c.
